(19) United States
(12) Patent Application Publication
(10) Pub. No.: US 2009/0209007 A1
(43) Pub. Date: Aug. 20, 2009

(54) Method of Expression Cloning in a Host Cell

(76) Inventors: Roelf Bernhard Meima, Kamerik (NL); Thibaut Jose Wenzel, Leiden (NL); Cornelis Maria Jacobus Sagt, Utrecht (NL); Johannes Hendrik Winde, Voorhout (NL); Oscar Paul Kuipers, Groningen (NL); Rutger Jan Van Rooijen, Ede (NL); Marjolein Cornelia Dekker, Rotterdam (NL); Brenda Vonk, Spijkenisse (NL)

Correspondence Address: NIXON & VANDERHYE, PC, 901 NORTH GLEBE ROAD, 11TH FLOOR, ARLINGTON, VA, 22203, US

(21) Appl. No.: 11/886,585

(22) PCT Filed: Mar. 20, 2006
(86) PCT No.: PCT/EP2006/060864

§ 371 Date: Sep. 18, 2007

(30) Foreign Application Priority Data
Mar. 18, 2005 (EP) 05102159.0
Mar. 18, 2005 (EP) 05102160.8
Mar. 18, 2005 (EP) 05102177.2
Mar. 18, 2005 (EP) 05102180.6
Mar. 18, 2005 (EP) 05102184.8

(51) Int. Cl. C12P 19/36; C07H 21/04; C12N 15/63; C12N 1/15; C12N 1/21

(52) U.S. Cl. 435/91.1; 536/24.1; 435/320.1; 435/252.5; 435/256.3; 435/256.1; 435/256.7

(57) ABSTRACT

The invention relates to a promoter DNA sequence highly suited in an improved expression cloning method for isolation of DNA sequences comprising a DNA sequence encoding a protein of interest in a host cell and to the improved expression cloning method wherein use is made of this promoter. The isolated DNA sequences are useful in processes for producing a protein of interest.

### FIELD OF THE INVENTION

The present invention relates to a promoter highly suited for being used in a method for the identification of DNA sequences encoding proteins of interest by expression cloning.

### BACKGROUND OF THE INVENTION

An increasing number of screening methods based on expression cloning are already known. Such methods have previously successfully been used for identification of prokaryotic gene products in e.g. Bacillus (cf. U.S. Pat. No. 4,469,791, WO2005/38024) and E. coli (e.g. WO 95/18219 and WO 95/34662). WO2005/38024 describes a method of screening protein secreting recombinant Bacillus cells. However, this method is lacking high throughput, automated detection technology. Yeast has also been used as a host for expression cloning of eukaryotic genes. Strasser et al. (Eur. J. Biochem. (1989)184: 699-706) have reported the identification of a fungal α-amylase by expression cloning of fungal genomic DNA in the yeast Saccharomyces cerevisiae. Similarly, WO 93/11249 reports the identification of a fungal cellulase by expression cloning of fungal cDNAs in S. cerevisiae. Lastly, an expression cloning method in filamentous fungus has been described (WO 99/32617).

In all these methods, large number of transformants would have to be screened before a transformant secreting a protein with properties of interest could be isolated. There is thus a need for an expression cloning method that would optimise the chance of detecting DNA sequences encoding secreted proteins.

### BRIEF DESCRIPTION OF THE FIGURES

FIG. 1. Depicted is pPhtrA-gfp-amyE. This secretion reporter vector comprises the htrA promoter of B. subtilis in operative association with GFP. This vector is representative for pPhtrB-gfp-amyE.

FIG. 2. Construction of B. subtilis reporter strains for secretion-stress. The htr promoters were fused to the GFP gene, finally resulting in plasmids pPhtrA-gfp-amyE and pPhtrB-gfp-amyE. The resulting plasmids were integrated into the chromosome of Bacillus host cells via single crossing-over recombination in the corresponding htr loci, resulting in VT210A and VT210B cells. Cmr represents the chloramphenicol resistance gene.

FIG. 3. Depicted is pUBnpr-2, comprising the neutral protease gene npr (Genbank accession number K 02497) from Bacillus amyloliquefaciens.

FIG. 4. Depicted is pUBBAG, comprising the β-glucanase gene bag (Genbank accession number M15674) from Bacillus amyloliquefaciens.

FIG. 5. Depicted is the analysis of Bacillus subtilis VT210A cells transformed with either plasmid pUB110 (empty control vector) or plasmid pKTH10 (encoding Bacillus amyloliquefaciens a-amylase gene amyQ) using Fluorescence Activated Cell Sorting. On the y-axis the amount of observed events is depicted, on the x-axis the relative fluorescence is depicted. In panel A, analysis of Bacillus subtilis VT210A cells transformed with empty vector pUB110 is depicted. In panel B, analysis of Bacillus subtilis VT210A cells transformed with amyQ vector pKTH10 is depicted. In panel C, analysis of a mixed population of Bacillus subtilis VT210A cells transformed with a ratio of pUB110 and pKTH10 of 1:20 is depicted. In panel D, cell sorting of a mixed population of Bacillus subtilis VT210A cells transformed with both pUB110 and pKTH10 is depicted; the cell sorting limit is indicated by a dashed vertical line. Cells demonstrating higher fluorescence than this limit were sorted.

FIG. 6. Depicted is the analysis of a mixed population of Bacillus subtilis VT210B cells transformed with either plasmid pUB110 (empty control vector) or plasmid pKTH10 (encoding Bacillus amyloliquefaciens a-amylase gene amyQ). Analysis was performed by Fluorescence Activated Cell Scanning. On the y-axis the amount of observed events is depicted, on the x-axis the relative fluorescence is depicted.

FIG. 7. Depicted is the pGBFINGFP-2 vector used for expression of Green Fluorescent Protein (GFP, Chalfie, M et al., Science (1994) 263(5148): 802-805) in A. niger. The backbone features of the depicted FIN vectors were previously described in WO 99/32617.

FIG. 8. Depicted is the pGBFIN-32 vector used for expression of phytase (PHY) (phytase gene PHY identical to FytA described in WO99/32617) in A. niger.

FIG. 9. Depicted is the pGBFIN-40 vector with the larger part of the phytase gene and the entire glucoamylase promoter removed, this vector is used as empty control vector for construction of A. niger WT-vector strain.

FIG. 10. Depicted is a Northern Blot analysis performed to confirm the identification of secretion-induced promoters.

FIG. 11. Depicted is a vector used for gene expression in A. niger. This vector is used as backbone in the construction of the secretion-induced A. niger reporter constructs.

FIG. 12. Depicted is the pGBFINGFPBLE-1 cloning vector. This secretion-inducible reporter construct comprises the GFP-BLE reporter fusion gene in operative association with secretion-inducible promoter 1 of the present invention. The construct is representative for pGBFINGFPBLE-2, pGBFINGFPBLE-3, pGBFINGFPBLE-4, and pGBFINGFPBLE-5.

FIG. 13. Depicted is pGBTOPSEL-1, this vector is used as the backbone for construction of the final secretion-inducible reporter plasmids. Backbone features of the depicted vector were previously described in WO 99/32617.

FIG. 14. Depicted is the final secretion reporter construct pGBTOPGFPBLE-1. This secretion-induced reporter vector comprises the GFP-BLE reporter construct in operative association with secretion-inducible promoter 1 of the present invention. The vector is representative for pGBTOPGFPBLE-2, pGBTOPGFPBLE-3, pGBTOPGFPBLE-4, and pGBTOPGFPBLE-5.

FIG. 15. Depicted is the difference in phleomycin resistance of A. niger co-transformants containing a reporter construct with secretion-inducible promoter P4 in combination with a library construct encoding either an intracellular protein (represented by black dots) or an extracellular protein (represented by black squares).

FIG. 16. Depicted is the difference in phleomycin resistance of A. niger co-transformants containing a reporter construct with secretion-inducible promoter P5 in combination with a library construct encoding either an intracellular protein (represented by black dots) or an extracellular protein (represented by black squares).

FIG. 17. Depicted is an E-PAGE™ gel, wherein each lane represents a co-transformed A. niger clone comprising a reporter contruct with secretion-inducible promoter P1 in combination with a library construct encoding an extracellular protein. M represents the E-PAGE SeeBlue® prestained standard (Invitrogen, U.K.).

FIG. 18. Depicted is an E-PAGE™ gel, wherein each lane represents a co-transformed A. niger clone comprising a reporter contruct with secretion-inducible promoter P3 in combination with a library construct encoding an extracellular protein. M represents the E-PAGE SeeBlue® prestained standard (Invitrogen, U.K.).

### DESCRIPTION OF THE INVENTION

### Promoter DNA Sequence

According to a first aspect of the invention, there is provided a promoter DNA sequence such as:
(a) a DNA sequence as presented in the following list: SEQ ID NO:1, SEQ ID NO:2, SEQ ID NO:20, SEQ ID NO:21, SEQ ID NO:22, SEQ ID NO:23 or SEQ ID NO:24,
(b) a DNA sequence capable of hybridizing with a DNA sequence of (a),
(c) a DNA sequence being at least 50% homologous to a DNA sequence of (a),
(d) a variant of any of the DNA sequences of (a) to (c), or
(e) a subsequence of any of the DNA sequences of (a) to (d).

According to a preferred embodiment, the promoter sequence of the invention is one promoter DNA sequence such as:
(a) one DNA sequence as presented in the following list: SEQ ID NO:1, SEQ ID NO:2, SEQ ID NO:20, SEQ ID NO:21, SEQ ID NO:22, SEQ ID NO:23 or SEQ ID NO:24,
(b) one DNA sequence capable of hybridizing with a DNA sequence of (a),
(c) one DNA sequence being at least 50% homologous to a DNA sequence of (a),
(d) one variant of any of the DNA sequences of (a) to (c), or
(e) one subsequence of any of the DNA sequences of (a) to (d).

In the context of this application, a promoter DNA sequence is a DNA sequence, which is capable of controlling the expression of a coding sequence, when this promoter DNA sequence is in operative association with this coding sequence. The term “in operative association” is defined herein as a configuration in which a promoter DNA sequence is appropriately placed at a position relative to a coding sequence such that the promoter DNA sequence directs the production of a polypeptide encoded by the coding sequence.

The term “coding sequence” is defined herein as a nucleic acid sequence that is transcribed into mRNA, which is translated into a polypeptide when placed under the control of the appropriate control sequences. The boundaries of the coding sequence are generally determined by the ATG start codon, which is normally the start of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences. Preferably, a promoter DNA sequence is defined by being the DNA sequence located upstream of a coding sequence associated thereto and by being capable of controlling the expression of this coding sequence.

More specifically, the term “promoter” is defined herein as a DNA sequence that binds the RNA polymerase and directs the polymerase to the correct downstream transcriptional start site of a coding sequence encoding a polypeptide to initiate transcription. RNA polymerase effectively catalyzes the assembly of messenger RNA complementary to the appropriate DNA strand of the coding region. The term “promoter” will also be understood to include the 5′ non-coding region (between promoter and translation start) for translation after transcription into mRNA, cis-acting transcription control elements such as enhancers, and other nucleotide sequences capable of interacting with transcription factors.

In a preferred embodiment, the promoter DNA sequence of the invention is a DNA sequence derived from: SEQ ID NO:1, SEQ ID NO:2, SEQ ID NO:20, SEQ ID NO:21, SEQ ID NO:22, SEQ ID NO:23 or SEQ ID NO:24.

According to another preferred embodiment, the promoter DNA sequence of the invention is a DNA sequence capable of hybridizing with a DNA sequence as presented in the following list: SEQ ID NO 1, SEQ ID NO:2, SEQ ID NO:20, SEQ ID NO:21, SEQ ID NO:22, SEQ ID NO:23 or SEQ ID NO:24 and which still retains promoter activity.

Promoter activity is preferably determined by measuring the concentration of the protein(s) produced as a result of the expression of a coding sequence(s), which is (are) in operative association with the promoter. Alternatively the promoter activity is determined by measuring the enzymatic activity of the protein(s) coded by the coding sequence(s), which is (are) in operative association with the promoter. According to a preferred embodiment, the promoter activity (and its strength) is determined by measuring the expression of the coding sequence of the lacZ reporter gene (In Luo, Gene 163 (1995) 127-131 or Perkins and Youngman (1986) Proc Natl Acad Sci USA 83: 140; or Vagner et al. (1998) Microbiology 144: 3097). According to another preferred embodiment, the promoter activity is determined by using the green fluorescent protein as coding sequence (In Microbiology. 1999 March; 145 (Pt 3):729-34. Santerre Henriksen A L, Even S, Muller C, Punt P J, van den Hondel C A, Nielsen J.Study). In bacterial cells, GFP and other fluorescent proteins have been used extensively for expression and (dynamic) protein localization studies (reviewed in Southward and Surette (2002) Mol. Microbiol. 45: 1191). Additionally, promoter activity can be determined by measuring the mRNA levels of the transcript generated under control of the promoter. The mRNA levels can, for example, be measured by Northern blot, GeneChips™ (Affymetrix) or spotted array technology or quantitative (real time) PCR (J. Sambrook, E. F. Fritsch, and T. Maniatis, 2001, Molecular Cloning, A Laboratory Manual, 3d edition, Cold Spring Harbor, N.Y.). Most preferably, promoter activity is determined by mRNA analysis using Northern blot.

Preferably, (isolated) promoter DNA sequences of the invention hybridize under very low stringency conditions, more preferably low stringency conditions, more preferably medium stringency conditions, more preferably medium-high stringency conditions, even more preferably high stringency conditions, and most preferably very high stringency conditions with a nucleic acid probe which hybridizes under the same conditions with:

### (i) SEQ ID NO:1, SEQ ID NO:2, SEQ ID NO:20, SEQ ID NO:21, SEQ ID NO:22, SEQ ID NO:23 or SEQ ID NO:24, or

(ii) a subsequence of (i), or
(iii) a complementary strand of (i), (ii),

The term complementary strand is known to the person skilled in the art and is described in (J. Sambrook, E. F. Fritsch, and T. Maniatus, 1989, Molecular Cloning, A Laboratory Manual, 2d edition, Cold Spring Harbor, N.Y.). A subsequence of SEQ ID NO:1, SEQ ID NO:2, SEQ ID NO:20, SEQ ID NO:21, SEQ ID NO:22, SEQ ID NO:23 or SEQ ID NO:24 may be ranged between 20 nucleotides and the respective whole sequence of SEQ ID NO:1, SEQ ID NO:2, SEQ ID NO:20, SEQ ID NO:21, SEQ ID NO:22, SEQ ID NO:23 or SEQ ID NO:24 upstream the coding region (the corresponding coding regions of SEQ ID NO:20, SEQ ID NO:21, SEQ ID NO:22, SEQ ID NO:23 or SEQ ID NO:24 are respectively given in SEQ ID NO:25, SEQ ID NO:26, SEQ ID NO:27, SEQ ID NO:28 and SEQ ID NO:29).

The nucleic acid sequence of SEQ ID NO:1, SEQ ID NO:2, SEQ ID NO:20, SEQ ID NO:21, SEQ ID NO:22, SEQ ID NO:23 or SEQ ID NO:24 or a subsequence thereof (as defined in the previous paragraph) may be used to design a nucleic acid probe to identify and clone DNA promoters from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Additionally, such probes can be used to amplify DNA promoters though PCR. Longer probes can also be used. DNA, RNA and Peptide Nucleid Acid (PNA) probes can be used. The probes are typically labeled for detecting the corresponding gene (for example, with @32 P, @33 P @3H, @35 S, biotin, or avidin or a fluorescent marker). Such probes are encompassed by the present invention.

Thus, a genomic DNA or cDNA library prepared from such other organisms may be screened for DNA, which hybridizes with the probes described above and which encodes a polypeptide. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA sequence which is homologous with SEQ ID NO:1, SEQ ID NO:2, SEQ ID NO:20, SEQ ID NO:21, SEQ ID NO:22, SEQ ID NO:23 or SEQ ID NO:24 or a subsequence thereof, the carrier material may be used in a Southern blot. For purposes of the present invention, hybridization indicates that the DNA sequence hybridizes under very low to very high stringency conditions to a labeled nucleic acid probe corresponding to the DNA sequence shown in SEQ ID NO:1, SEQ ID NO:2, SEQ ID NO:20, SEQ ID NO:21, SEQ ID NO:22, SEQ ID NO:23 or SEQ ID NO:24, to the complementary strands of said DNA sequences, or to subsequences of said DNA sequences. Molecules to which the nucleic acid probe hybridizes under these conditions are detected using for example a X-ray film. Other hybridisation techniques also can be used, such as techniques using fluorescence for detection and glass sides and/or DNA microarrays as support. An example of DNA microarray hybridisation detection is given in FEMS Yeast Res. 2003 December; 4(3):259-69 (Daran-Lapujade P, Daran J M, Kotter P, Petit T, Piper M D, Pronk J T. “Comparative genotyping of the Saccharomyces cerevisiae laboratory strains S288C and CEN.PK113-7D using oligonucleotide microarrays”. Additionally, the use of PNA microarrays for hybridization is described in Nucleic Acids Res. 2003 October 1; 31(19):e119 (Brandt O, Feldner J, Stephan A, Schroder M, Schnolzer M, Arlinghaus H F, Hoheisel J D, Jacob A. PNA microarrays for hybridisation of unlabelled DNA samples.)

In a preferred embodiment, the nucleic acid or DNA probe is the DNA sequence of SEQ ID NO:1, SEQ ID NO:2, SEQ ID NO:20, SEQ ID NO:21, SEQ ID NO:22, SEQ ID NO:23 or SEQ ID NO:24. In another embodiment, the nucleic acid probe is the DNA sequence having nucleotides 100 to 150 of SEQ ID NO:1 or the DNA sequence having nucleotides 170 to 220 of SEQ ID NO:2.

In another preferred embodiment, the nucleic acid probe is the DNA sequence having:
nucleotides 996 to 2797 of SEQ ID NO: 20, nucleotides 996 to 3561 of SEQ ID NO: 21, nucleotides 997 to 3536 of SEQ ID NO: 22, nucleotides 1034 to 3175 of SEQ ID NO: 23, or nucleotides 1080 to 4054 of SEQ ID NO: 24, more preferably:
nucleotides 1496 to 2497 of SEQ ID NO: 20, nucleotides 1496 to 3261 of SEQ ID NO: 21, nucleotides 1497 to 3236 of SEQ ID NO: 22, nucleotides 1534 to 2875 of SEQ ID NO: 23, or nucleotides 1580 to 3754 of SEQ ID NO: 24, even more preferably:
nucleotides 1896 to 2197 of SEQ ID NO: 20, nucleotides 1896 to 2861 of SEQ ID NO: 21, nucleotides 1897 to 2936 of SEQ ID NO: 22, nucleotides 1934 to 2575 of SEQ ID NO: 23, or nucleotides 1980 to 3454 of SEQ ID NO: 24, and most preferably:
nucleotides 1996 to 2100 of SEQ ID NO: 20, nucleotides 1996 to 2300 of SEQ ID NO: 21, nucleotides 1997 to 2400 of SEQ ID NO: 22, nucleotides 2034 to 2100 of SEQ ID NO: 23, or nucleotides 2080 to 2800 of SEQ ID NO: 24.

According to another preferred embodiment, the probe is part of the DNA sequence of SEQ ID NO:20, SEQ ID NO:21, SEQ ID NO:22, SEQ ID NO:23 or SEQ ID NO:24 located upstream of the coding sequence. The corresponding coding regions of SEQ ID NO:20, SEQ ID NO:21, SEQ ID NO:22, SEQ ID NO:23 or SEQ ID NO:24 are respectively given in SEQ ID NO:25, SEQ ID NO:26, SEQ ID NO:27, SEQ ID NO:28 and SEQ ID NO:29).

For long probes of at least 100 nucleotides in length, very low to very high stringency conditions are defined as prehybridization and hybridization at 42 degrees Celsius in 5 times SSPE, 0.3% SDS, 200 microgram/ml sheared and denatured salmon sperm DNA, and either 25% formamide for very low and low stringencies, 35% formamide for medium and medium-high stringencies, or 50% formamide for high and very high stringencies, following standard Southern blotting procedures. For long probes of at least 100 nucleotides in length, the carrier material is finally washed three times each for 15 minutes using 2 times SSC, 0.2% SDS preferably at least at 45 DEG C. (very low stringency), more preferably at least at 50 degrees Celsius (low stringency), more preferably at least at 55 degrees Celsius (medium stringency), more preferably at least at 60 degrees Celsius (medium-high stringency), even more preferably at least at 65 degrees Celsius (high stringency), and most preferably at least at 70 degrees Celsius (very high stringency).

For short probes which are about 15 nucleotides to about 70 nucleotides in length, stringency conditions are defined as prehybridization, hybridization, and washing post-hybridization at 5 degrees Celsius to 10 degrees Celsius below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1.times.Denhardt's solution, 1 mM sodium pyrophosphate, 1 mM sodium monobasic phosphate, 0.1 mM ATP, and 0.2 mg of yeast RNA per ml following standard Southern blotting procedures. For short probes, which are about 15 nucleotides to about 70 nucleotides in length, the carrier material is washed once in 6 times SCC plus 0.1% SDS for 15 minutes and twice each for 15 minutes using 6 times SSC at 5 degrees Celsius to 10 degrees Celsius below the calculated Tm.

According to another preferred embodiment, the promoter DNA sequence of the invention derived from SEQ ID NO:1, SEQ ID NO:2, SEQ ID NO:20, SEQ ID NO:21, SEQ ID NO:22, SEQ ID NO:23 or SEQ ID NO:24 is first used to clone the native gene, coding sequence or part of it, which is operatively associated with it. This can be done starting with either SEQ ID NO:1, SEQ ID NO:2, SEQ ID NO:20, SEQ ID NO:21, SEQ ID NO:22, SEQ ID NO:23 or SEQ ID NO:24 or a subsequence thereof as earlier defined and using this sequence as a probe. The probe is hybridised to a cDNA or a genomic library of a given host either Bacillus, Aspergillus niger or any other host as defined in this application. Once the native gene or part of it has been cloned, it can be subsequently used itself as a probe to clone homologous genes thereof derived from other host by hybridisation experiments as described herein. In this context, a homologous gene means a gene, which is at least 50% homologous to the native gene. Preferably, the homologous gene is at least 55% homologous, more preferably at least 60%, more preferably at least 65%, more preferably at least 70%, even more preferably at least 75% preferably about 80%, more preferably about 90%, even more preferably about 95%, and most preferably about 97% homologous to the native gene.

The sequence upstream the coding sequence of the homologous gene is a promoter encompassed by the present invention. Alternatively, the sequence of the native gene, coding sequence or part of it, which is operatively associated with a promoter of the invention can be identified by using SEQ ID NO:25, SEQ ID NO:26, SEQ ID NO:27, SEQ ID NO:28 or SEQ ID NO:29 or a subsequence thereof as earlier defined to search genomic databases using for example an alignment or BLAST algorithm as described herein. This identified sequence subsequently can be used to identify orthologues or homologous genes in any other fungal host as defined in this application. The sequence upstream the coding sequence of the identified orthologue or homologous gene is a promoter encompassed by the present invention.

According to another preferred embodiment, the promoter DNA sequence of the invention is a(n) (isolated) DNA sequence derived from SEQ ID NO:1, SEQ ID NO:2, SEQ ID NO:20, SEQ ID NO:21, SEQ ID NO:22, SEQ ID NO:23 or SEQ ID NO:24 and which still has promoter activity as defined earlier.

According to another preferred embodiment, the promoter DNA sequence of the invention is a(n) (isolated) DNA sequence derived from either SEQ ID NO:20, SEQ ID NO:21, SEQ ID NO:22, SEQ ID NO:23 or SEQ ID NO:24, respectively, which is at least 50% homologous to at least part of the respective corresponding DNA sequences: SEQ ID NO:15, SEQ ID NO:16, SEQ ID NO:17, SEQ ID NO:18 or SEQ ID NO:19 situated upstream of the respective coding regions (SEQ ID NO:25, SEQ ID NO:26, SEQ ID NO:27, SEQ ID NO:28 or SEQ ID NO:29). Preferably, the derived DNA sequence is at least 55% homologous, more preferably at least 60%, more preferably at least 65%, more preferably at least 70%, even more preferably at least 75% preferably about 80%, more preferably about 90%, even more preferably about 95%, and most preferably about 97% homologous to at least part of SEQ ID NO:15, SEQ ID NO:16, SEQ ID NO:17, SEQ ID NO:18 or SEQ ID NO:19 situated upstream of the respective corresponding coding region.

For purposes of the present invention, the degree of homology between two nucleic acid sequences is preferably determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=3, gap penalty=3, and windows=20.

According to another preferred embodiment, the promoter DNA sequence of the invention is derived from a(n) (isolated) DNA sequence, which is a variant of either SEQ ID NO:1, SEQ ID NO:2, SEQ ID NO:15, SEQ ID NO:16, SEQ ID NO:17, SEQ ID NO:18 or SEQ ID NO:19 or part of SEQ ID NO:1, SEQ ID NO:2, SEQ ID NO:15, SEQ ID NO:16, SEQ ID NO:17, SEQ ID NO:18 or SEQ ID NO:19 situated upstream of the respective corresponding coding region. The term “variant promoter” is defined herein as a promoter having a nucleotide sequence comprising a substitution, deletion, and/or insertion of one or more nucleotides of a parent promoter, wherein the variant promoter has more or less promoter activity than the corresponding parent promoter. The term “variant promoter” will also encompass natural variants and in vitro generated variants obtained using methods well known in the art such as classical mutagenesis, site-directed mutagenesis, and DNA shuffling. A variant promoter may have one or more mutations. Each mutation is an independent substitution, deletion, and/or insertion of a nucleotide. The introduction of a substitution, deletion, and/or insertion of a nucleotide into the promoter may be accomplished using any of the methods known in the art such as classical mutagenesis, site-directed mutagenesis, or DNA shuffling. Particularly useful is a procedure, which utilizes a supercoiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with Dpnl, which is specific for methylated and hemimethylated DNA to digest the parental DNA template and to select for mutation-containing synthesized DNA. Other procedures known in the art may also be used. Example of other procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8 1995 Horizon Scientific Press, PO Box 1, Wymondham, Norfolk, U.K.). According to a preferred embodiment, the variant promoter is a promoter, which has at least one modified regulatory site as compared to the promoter sequence first derived from SEQ ID NO:1, SEQ ID NO:2, SEQ ID NO:15, SEQ ID NO:16, SEQ ID NO:17, SEQ ID NO:18 or SEQ ID NO:19. Such regulatory site can have been removed in its entirety or specifically mutated as explained above. The regulation of such promoter variant is thus modified so that for example it is no longer induced by glucose. Examples of such promoter variants and techniques on how to obtain them are described in EP 673 429B or in WO 94/04673.

The promoter variant can be an allelic variant. An allelic variant denotes any of two or more alternative forms of a gene occupying the same chomosomal locus. Allelic variation arises naturally through mutation, and may result in polymorphism within populations. The variant promoter may be obtained by the following method comprising steps (a) and (b):

(a) hybridizing a DNA sequence under very low, low, medium, medium-high, high, or very high stringency conditions with:
(i) SEQ ID NO:1, SEQ ID NO:2, SEQ ID NO:15, SEQ ID NO:16, SEQ ID NO:17, SEQ ID NO:18 or SEQ ID NO:19, or
(ii) a subsequence of (i), or
(iii) a complementary strand of (i), (ii), and
   (b) isolating the variant promoter from the DNA sequence. Stringency and wash conditions are defined herein.

The variant promoter can be a promoter, whose sequence may be provided with linkers for the purpose of introducing specific restriction sites facilitating ligation of the promoter sequence with the coding region of the nucleic acid sequence encoding a polypeptide.

In another preferred embodiment, the promoter DNA sequence is derived from a subsequence of SEQ ID NO:1, SEQ ID NO:2, SEQ ID NO:15, SEQ ID NO:16, SEQ ID NO:17, SEQ ID NO:18 or SEQ ID NO:19. A subsequence is preferably defined in this context as being part of SEQ ID NO:1, SEQ ID NO:2, SEQ ID NO:15, SEQ ID NO:16, SEQ ID NO:17, SEQ ID NO:18 or SEQ ID NO:19 located upstream of the respective corresponding coding sequence as defined previously.

The subsequence preferably contains at least about 50 nucleotides, or preferably at least about 100 nucleotides, more preferably at least about 200 nucleotides, even more preferably at least about 230 nucleotides, even more preferably at least about 250 nucleotides, and most preferably at least about 290 nucleotides.

According to another preferred embodiment, the subsequence differs from the one defined in former sentence by the fact that one or more nucleotides from the 5′ and/or 3′ end have been deleted, said DNA sequence still having promoter activity as defined earlier.

In another preferred embodiment, the promoter subsequence is a ‘trimmed’ subsequence from translation start and/or from transcription start. An example of trimming a promoter and functionally analysing it is described in Gene. 1994 Aug. 5; 145(2):179-87: the effect of multiple copies of the upstream region on expression of the Aspergillus niger glucoamylase-encoding gene. Verdoes J C, Punt P J, Stouthamer A H, van den Hondel C A).

The sequence information as provided herein should not be so narrowly construed as to require inclusion of erroneously identified bases. The specific sequences disclosed herein can be readily used and be eventually subjected to further sequence analyses thereby identifying sequencing errors to isolate the original DNA sequence from a filamentous fungus, in particular Aspergillus niger.

Unless otherwise indicated, all nucleotide sequences determined by sequencing a DNA molecule herein were determined using an automated DNA sequencer. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art.

The person skilled in the art is capable of identifying such erroneously identified bases and knows how to correct for such errors.

Functional nucleic acid equivalents may typically contain mutations that do not alter the biological function of the promoter it contains. The term “functional equivalents” also encompasses orthologues of the A. niger DNA sequences. Orthologues of the A. niger DNA sequences are DNA sequences that can be isolated from other strains or species and possess a similar or identical biological activity.

Homologous (similar or identical) sequences can also be determined by using a “sequence comparison algorithm”. Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. An example of an algorithm that is suitable for determining sequence similarity is the BLAST algorithm, which is described in Altschul, et al., J. Mol. Biol. 215: 403-410 (1990). Alternatively other programs can be used for sequence alignment as earlier described herein. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89: 10915 (1989)) alignments (B) of 50, expectation (E) of 10, M=5, N=−4, and a comparison of both strands. The BLAST algorithm then performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a protein such as a protease if the smallest sum probability in a comparison of the test amino acid sequence to a protein such as a protease amino acid sequence is less than about 0.1, more preferably less than about 0.01, and most preferably less than about 0.001.

Preferably the similarity of the variant promoter is at least 40% homology to one of the DNA sequences having SEQ ID NO:1, SEQ ID NO:2, SEQ ID NO:15, SEQ ID NO:16, SEQ ID NO:17, SEQ ID NO:18 or SEQ ID NO:19. More preferably the similarity is at least 50%, more preferably, at least 60%, more preferably at least 70%, more preferably at least 80%, more preferably at least 90% and more preferably at least 95% or at least 98% homology to the DNA sequence having SEQ ID NO:1, SEQ ID NO:2, SEQ ID NO:15, SEQ ID NO:16, SEQ ID NO:17, SEQ ID NO:18 or SEQ ID NO:19.

In addition to naturally occurring allelic variants of the promoter sequence, the skilled person will recognise that changes can be introduced by mutation into the promoter sequence derived from SEQ ID NO:1, SEQ ID NO:2, SEQ ID NO:15, SEQ ID NO:16, SEQ ID NO:17, SEQ ID NO:18 or SEQ ID NO:19, without substantially altering its promoter function.

The promoter sequences of the present invention may be obtained from microorganisms of any genus. For purposes of the present invention, the term “obtained from” as used herein in connection with a given source shall mean that the polypeptide is produced by the source or by a cell in which a gene from the source has been inserted. Preferably, the microorganism is a prokaryote. Preferred prokaryotes are Bacillus and E. coli. Preferred Bacilli are Bacillus subtilis, Bacillus amyloliquefaciens, Bacillus licheniformis, Bacillus alcalophilus, Bacillus clausii, Bacillus brevis, Bacillus circulans, Bacillus firmus, Bacilluspumilis, Bacillus stearothermophilus, Bacillus megaterium, Bacillus lentus, or Bacillus thuringiensis.

Alternatively, the promoter sequence is obtained from an eukaryote, preferably a fungal source, and more preferably from a yeast strain such as a Candida, Hansenula, Kluyveromyces, Pichia, Saccharomyces, Schizosaccharomyces, or Yarrowia strain; or more preferably from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain.

In a preferred embodiment, the promoter sequences are obtained from a Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces bayanus, Saccharomyces bayanus, var. uvarum or Saccharomyces pastorianus strain.

In another preferred embodiment, the promoter sequences are obtained from an Aspergillus aculeatus, Aspergillus awamori, Aspergillus foetidus, Aspergillus japonicus, A. nidulans, A. niger, preferably A. niger CBS 513.88, A. sojae, Aspergillus oryzae (A. oryzae), Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

In another preferred embodiment, the promoter sequences are obtained from a Fusarium bactridioides, Fusarium cerealis, Fusarium crookwellense, Fusarium culmorum, Fusarium graminearum, Fusarium graminum, Fusarium heterosporum Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum strain.

It will be understood that for the aforementioned species, the invention encompasses the perfect and imperfect states, and other taxonomic equivalents, e.g., anamorphs, regardless of the species name by which they are known. Those skilled in the art will readily recognize the identity of appropriate equivalents. Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such nucleic acid sequences may be identified and obtained from other sources including microorganisms isolated from nature (e.g, soil, composts, water, etc.) using the above-mentioned probes. Techniques for isolating microorganisms from natural habitats are well known in the art. The nucleic acid sequence may then be derived by similarly screening a genomic DNA library of another microorganism. Once a nucleic acid sequence encoding a promoter has been detected with the probe(s), the sequence may be isolated or cloned by utilizing techniques which are known to those of ordinary skill in the art (see, e.g., Sambrook et al., 1989, supra).

In the present invention, the promoter DNA sequence may also be a hybrid promoter comprising a portion of one or more promoters of the present invention; a portion of a promoter of the present invention and a portion of another known promoter, e.g., a leader sequence of one promoter and the transcription start site from the other promoter; or a portion of one or more promoters of the present invention and a portion of one or more other promoters. The other promoter may be any promoter sequence, which shows transcriptional activity in the host cell of choice including a variant, truncated, and hybrid promoter, and may be obtained from genes encoding extracellular or intracellular polypeptides either homologous or heterologous to the host cell. The other promoter sequence may be native or foreign to the nucleic acid sequence encoding the polypeptide and native or foreign to the cell.

As a preferred embodiment, important regulatory subsequences of the promoter identified can be fused to other ‘basic’ promoters to enhance their promoter activity (as for example described in Mol. Microbiol. 1994 May; 12(3):479-90. Regulation of the xylanase-encoding xInA gene of Aspergillus tubigensis. de Graaff L H, van den Broeck H C, van Ooijen A J, Visser J.).

Other examples of other promoters useful in the construction of hybrid promoters with the promoters of the present invention include the promoters obtained from the genes for A. oryzae TAKA amylase, Rhizomucor miehei aspartic proteinase, A. niger neutral alpha-amylase, A. niger acid stable alpha-amylase, A. niger or Aspergillus awamori glucoamylase (glaA), A. niger gpdA, A. niger glucose oxidase goxc, Rhizomucor miehei lipase, A. oryzae alkaline protease, A. oryzae triose phosphate isomerase, A. nidulans acetamidase, and Fusarium oxysporum trypsin-like protease (WO 96/00787), as well as the NA2-tpi promoter (a hybrid of the promoters from the genes for A. niger neutral alpha-amylase and A. oryzae triose phosphate isomerase), Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, and mutant, truncated, and hybrid promoters thereof. Other useful promoters for yeast host cells are described by Romanoset al., 1992, Yeast 8: 423-488. Other useful promoters for bacterial cells are the promoter of the following genes B. subtilis alkaline protease (apr, B. subtilis neutral protease (npr, B. amyloliquefaciens α-amylase (amyQ), B. amyloliquefaciens alkaline protease (apr, and B. amyloliquefaciens neutral protease (npr)

In the present invention, the promoter DNA sequence may also be a “tandem promoter”. A “tandem promoter” is defined herein as two or more promoter sequences each of which is in operative association with a coding sequence and mediates the transcription of the coding sequence into mRNA.

The tandem promoter comprises two or more promoters of the present invention or alternatively one or more promoters of the present invention and one or more other known promoters, such as those exemplified above useful for the construction of hybrid promoters. The two or more promoter sequences of the tandem promoter may simultaneously promote the transcription of the nucleic acid sequence. Alternatively, one or more of the promoter sequences of the tandem promoter may promote the transcription of the nucleic acid sequence at different stages of growth of the cell or, in the case of fungal hosts, morphological different parts of the mycelia, or different cellular compartments during spore development in sporulating bacteria such as bacilli.

In the present invention, the promoter may be foreign to the coding sequence and/or to the host cell. A variant, hybrid, or tandem promoter of the present invention will be understood to be foreign to a DNA sequence encoding a polypeptide even if the wild-type promoter is native to the coding sequence or to the host cell.

A variant, hybrid, or tandem promoter of the present invention has at least about 20%, preferably at least about 40%, more preferably at least about 60%, more preferably at least about 80%, more preferably at least about 90%, more preferably at least about 100%, even more preferably at least about 200%, most preferably at least about 300%, and even most preferably at least about 400% of the promoter activity of any promoter derived from SEQ ID NO:1, SEQ ID NO:2, SEQ ID NO:20, SEQ ID NO:21, SEQ ID NO:22, SEQ ID NO:23 or SEQ ID NO:24 and defined earlier. The promoter activity is preferably determined as defined earlier. Most preferably, promoter activity is determined by mRNA analysis using Northern blot.

### DNA Construct

According to a second aspect, the invention provides a DNA construct comprising a promoter DNA sequence of the invention as defined in the former section operatively associated with a reporter gene conferring a selectable trait. The reporter gene can be any gene conferring a selectable trait to a suitable host. Preferably, the reporter gene is a selection marker gene.

“DNA construct” is defined herein as a nucleic acid molecule, either single or double-stranded, which is isolated from a naturally occurring gene or which has been modified to contain segments of nucleic acid combined and juxtaposed in a manner that would not otherwise exist in nature.

The term DNA construct is synonymous with the term expression cassette when the DNA construct contains a coding sequence or reporter gene and all the control sequences required for expression of the coding sequence or reporter gene. In the context of this application the term “DNA contruct” is interchangeably used with the term “isolated DNA construct” wherein both terms are contrued to be synonymous.

The DNA construct may comprise a selection marker gene, which permits easy selection of transformed host cells. In case the DNA construct comprises two selection marker genes, the skilled person will understand that these two selection marker genes are not identical: the first one associated with the promoter of the invention is used in the screening method of the invention and the second one present on the DNA construct allows a standard selection of transformed host cells.

The reporter gene is in operative association with the promoter DNA sequence of the invention such that the reporter gene can be expressed under the control of the promoter DNA sequence in a given host cell. The polypeptide encoded by the reporter gene may be native or heterologous to the host cell and/or to the promoter DNA sequence.

In this context “a” means “at least one”. Therefore, the DNA construct comprises at least one reporter gene. The host may be co-transformed with at least two DNA constructs, one comprising the selection marker and another one comprising the promoter of the invention in association with a reporter gene.

Alternatively, and according to a more preferred embodiment, the reporter gene comprised in the DNA construct is a hybrid reporter gene comprising at least part of a first reporter gene with at least part of a second reporter gene, wherein the coding sequences of the first and second reporter genes are in-frame coupled to each other and wherein said hybrid reporter gene is operably linked to the promoter of the invention. The first and/or second reporter gene may be any gene conferring a selectable trait to a suitable host. A preferred hybrid reporter gene for fungi comprises the GFP gene and the phleomycin resistance gene (BLE) of Streptoalloteichus hindustanis (Drocourt et al., NAR, 1990).

A selection marker or selectable marker is a gene, the product of which provides for biocide or viral resistance, resistance to heavy metals, prototrophy to auxotrophs, and the like. Suitable selectable markers for prokaryotic host cells include, but are not limited to, kanamycin, neomycin, erythromycin and other MLS-type markers (macrolide- lincosamide-streptogramin B), chloramphenicol, ampicillin, tetracyclin, streptomycin and spectinomycin. Suitable markers for yeast host cells are ADE2, HIS3, LEU2, LYS2, MET3, TRP1, and URA3. Selectable markers for use in a flamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Marker conferring resistance against e.g. phleomycin, hygromycin B or G418 can also be used. Preferred for use in an Aspergillus cell are the amdS and pyrG genes of A. nidulans or A. oryzae and the bar gene of Streptomyces hygroscopicus. The amdS marker gene is preferably used applying the technique described in EP 635 574B or WO 97/06261. A preferred selection marker gene is the A. nidulans amdS coding sequence fused to the A. nidulans gpdA promoter (EP635 574B). AmdS genes from other filamentous fungus may also be used (WO 97/06261).

According to another preferred embodiment, the reporter gene encodes a selectable marker for prokaryotic, yeast and/or filamentous fungal cells as described here above. Alternatively, the reporter gene encodes a reporter protein like: beta-galactosidase, beta-glucuronidase, aequorin, Green Fluorescent Protein (GFP) and variants thereof (Red, Cyan, Yellow-fluorescent protein), luciferase, lux, heme, beta-lactamase, or alkaline phosphatase. More preferably, the reporter gene is or comprises a gene encoding a fluorescent reporter protein such as, but not limited to GFP, YFP, and CFP (green, yellow and cyan fluorescent proteins, respectively). Transformants will exhibit fluorescence and can be separated from non expressing clones using sensitive cell sorting technology (FACS, fluorescence activated cell sorting). An important advantage of this method is that plating of transformation mixtures is not required (which considerably increases the throughput of the screening procedure) and that positive clones can arrayed directly into an MTP format for further automated processing. Alternatively, the reporter gene encodes a (trans)membrane protein or a cell wall protein to which antibodies can be raised. Transformants exhibiting said membrane protein can be separated from non expressing transformants using magnetic cell sorting technologies (MACS®, Magnetic cell sorting, www.miltenyibiotec.com).

### Control Elements

The DNA construct may further comprise one or more control sequences in addition to the promoter DNA sequence, which direct the expression of the reporter gene in a suitable host cell under conditions compatible with the control sequences. Expression will be understood to include any step involved in the production of the polypeptide including, but not limited to, transcription, post-transcriptional modification, translation, post-translational modification, and secretion. One or more control sequences may be native to the reporter gene or to the host. Alternatively, one or more control sequences may be replaced with one or more control sequences foreign to the nucleic acid sequence for improving expression of the reporter gene in a host cell.

The term “control sequences” is defined herein to include all components, including the promoter of the invention, which are necessary or advantageous for the expression of a coding sequence such as a reporter gene. Each control sequence may be native or foreign to the coding sequence encoding a polypeptide. Such control sequences include, but are not limited to, a leader, optimal translation initiation sequences (as described in Kozak, 1991, J. Biol. Chem. 266:19867-19870), polyadenylation sequence, propeptide sequence, signal peptide sequence, upstream activating sequence, the promoter of the invention including variants, fragments, hybrid and tandem thereof and transcription terminator. At a minimum, the control sequences include transcriptional and translational stop signals and (part of) the promoter of the invention. The control sequences may be provided with linkers for the purpose of introducing specific restriction sites facilitating ligation of the control sequences with the coding sequence, such as a reporter gene, encoding a polypeptide.

The control sequence may be a suitable transcription terminator sequence, a sequence recognized by a host cell to terminate transcription. The terminator sequence is in operative association with the 3′ terminus of the coding sequence encoding the polypeptide. Any terminator, which is functional in the host cell of choice may be used in the present invention.

The skilled person would know that 5′ end and 3′ end of a sequence is defined for each coding sequence. For a given terminator sequence and a given coding sequence, the skilled person would know how to operatively associate them together.

Preferred terminators for filamentous fungal host cells are obtained from the genes for A. oryzae TAKA amylase, A. niger glucoamylase, A. nidulans anthranilate synthase, A. niger alpha-glucosidase, trpc gene, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al, 1992, supra. The control sequence may also be a suitable leader sequence, a nontranslated region of an mRNA which is important for translation by the host cell. The leader sequence is in operative association with the 5′ terminus of the nucleic acid sequence encoding the polypeptide. Any leader sequence that is functional in the host cell of choice may be used in the present invention.

Preferred leaders for filamentous fungal host cells are obtained from the genes for A. oryzae TAKA amylase, A. nidulans triose phosphateisomerase and A. niger glucoamylase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase and glyceraldehyde-3-phosphate dehydrogenase (ADH2 and GAP).

The control sequence may also be a polyadenylation sequence, a sequence in operative association with the 3′ terminus of the nucleic acid sequence and which, when Preferred polyadenylation sequences for filamentous fungal host cells are obtained from the genes for A. oryzae TAKA amylase, A. niger glucoamylase, A. nidulans anthranilate synthase, Fusarium oxysporum trypsin-like protease, and A. niger alpha-glucosidase.

Useful polyadenylation sequences for yeast host cells are described by Guo and Sherman, 1995, Molecular Cellular Biology 15: 5983-5990.

It may also be desirable to add regulatory sequences, which allow the regulation of the expression of the reporter gene relative to the growth of the host cell. Examples of regulatory systems are those which cause the expression of the reporter gene to be turned on or off in response to a chemical or physical stimulus, including the presence of a regulatory compound. Regulatory systems in prokaryotic systems include the lac, and trp operator systems of E. coli, and the spac, xyl, sacB, and citM systems in B. subtilis (reviewed in Meima et al. (2004) Expression Systems in Bacillus. In “Protein Expression Technologies. Current status and future trends” (F. Baneyx, ed.), Horizon Bioscience, Wymondham, Norfolk, UK, pp. 199-252). Other promoters useful for controlled expression in bacilli are those induced under nutrient starvation conditions such as the Pho regulon and TnrA/GlnR system, which are under control of phosphate aid nitrogen, respectively. In yeast, the ADH2 system or GALL system may be used. In filamentous fungi, the TAKA alpha-amylase promoter, A. niger glucoamylase promoter, A. oryzae glucoamylase promoter, A. tubingensis endoxylanase (xlnA) promoter, A. niger nitrate reductase (niaD) promoter, Trichoderma reesei cellobiohydrolase promoter and the A. nidulans alcohol and aldehyde dehydrogenase (alcA and aldA, respectively) promoters as described in U.S. Pat. No. 5,503,991) may be used as regulatory sequences. Other examples of regulatory sequences are those, which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the polypeptide would be in operative association with the regulatory sequence.

Endogenous regulatory sequence(s) present in the promoter DNA sequence of the invention may be removed, for example removal of creA binding sites (carbon catabolite repression as described earlier in EP673429B), change of pacC and areA (for pH and nitrogen regulation). In B. subtilis, carbon catabolite repression (CCR) involves binding of the CcpA protein to the cre element. The cre element was used in combination with the xylose inducible xyl promoter for the development of an expression system, allowing for dual control of genes of interest (Bhavsar et al. (2001) Appl Environ Microbiol 67: 403).

Preferably, the DNA construct comprises a promoter DNA sequence from the invention, a reporter gene in operative association with said promoter DNA sequence and translational control sequences such as:
one translational termination sequence orientated in 5′ towards 3′ direction selected from the following list of sequences: TAAG, TAGA and TAAA, preferably TAM, and/or
one translational initiator coding sequence orientated in 5′ towards 3′ direction selected from the following list of sequences: GCTACCCCC; GCTACCTCC; GCTACCCTC; GCTACCTTC; GCTCCCCCC; GCTCCCTCC; GCTCCCCTC; GCTCCCTTC; GCTGCCCCC; GCTGCCTCC; GCTGCCCTC; GCTGCCTTC; GCTTCCCCC; GCTTCCTCC; GCTTCCCTC; and GCTTCCTTC, preferably GCT TCC TTC, and/or
one transcriptional initiator sequence selected from the following list of sequences: 5′-mwChkyCAAA-3′; 5′-mwChkyCACA-3′ or 5′-mwChkyCMG-3′, using ambiguity codes for nucleotides: m (A/C); w (A/T); y (C/T); k (G/T); h (A/C/T), preferably 5′-CACCGTCAAA-3′ or 5′-CGCAGTCAAG-3′.

In the context of this invention, the term “translational initiator coding sequence” is defined as the nine nucleotides immediately downstream of the initiator or start codon of the open reading frame of a DNA coding sequence. The initiator or start codon encodes for the AA methionine. The initiator codon is typically ATG, but may also be any functional start codon such as GTG.

In the context of this invention, the term “translational termination sequence” is defined as the three or four nucleotides starting from the translational stop codon at the 3′ end of the open reading frame or nucleotide coding sequence and oriented in 5′ towards 3′ direction.

In the context of this invention, the term “translational initiator sequence” is defined as the ten nucleotides immediately upstream of the initiator or start codon of the open reading frame of a DNA sequence coding for a polypeptide. The initiator or start codon encodes for the AA methionine. The initiator codon is typically ATG, but may also be any functional start codon such as GTG. It is well known in the art that uracil, U, replaces the deoxynucleotide thymine, T, in RNA.

Preferred control sequences for prokaryotes have already been described in EP 284 126A.

The present invention also relates to recombinant expression vectors comprising a promoter of the present invention operatively associated with a reporter gene, and transcriptional and translational stop signals. The various reporter gene and control sequences described above may be joined together to produce a recombinant expression vector which may include one or more convenient restriction sites to allow for insertion or substitution of the promoter and/or reporter gene at such sites. Alternatively, fusion of reporter gene and promoter can be done by e.g. sequence overlap extension using PCR (SOE-PCR), as described in Gene. 1989 Apr. 15; 77(1):51-9. Ho S N, Hunt H D, Horton R M, Pullen J K, Pease L R “Site-directed mutagenesis by overlap extension using the polymerase chain reaction”) or by cloning using the Gateway™ cloning system (Invitrogen).

The recombinant expression vector may be any vector capable of transforming a host cell. The choice of the vector will typically depend on the compatibility of the vector with the host cell into which the vector is to be introduced. The vectors may be linear or closed circular plasmids. The vectors may be integrative or autonomously replicating vectors.

The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. The autonomously replicating vector may contain any means for assuring self-replication. The vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. An example of an autonomously maintained cloning vector is a cloning vector comprising the AMA1-sequence. AMAL is a 6.0-kb genomic DNA fragment isolated from A. nidulans, which is capable of Autonomous Maintenance in Aspergillus (see e.g. Aleksenko and Clutterbuck (1997), Fungal Genet. Biol. 21: 373-397). Examples of autonomously replicating vectors useful for expression in Gam-positive bacteria such as B. subtilis and B. amyloliquefaciens are the staphylococcal vector pUB110 (McKenzie et al. (1986) Plasmid 15: 93) and the endogenous B. subtilis plasmids pTA1015, pTA1040 and pTA1060 and vectors derived thereof (e.g. pHB201; Bron et al. (1998) J Biotechnol 64: 3).

The vectors of the present invention preferably contain (an) element(s) that permits stable integration of the vector into the host cell's genome or autonomous replication of the vector in the cell independent of the genome.

For integration into the host cell genome, the vector may rely on the promoter sequence and/or reporter gene sequence or any other element of the vector for stable integration of the vector into the genome by homologous or non-homologous recombination. The vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 20 to 50, preferably, 55 to 95, preferably 100 to 1,500 base pairs, preferably 400 to 1,500 base pairs, more preferably 800 to 1,500 base pairs, and most preferably at least 2 kb, which are homologous to a DNA sequence in a predetermined target locus in the genome of the host cell. The integrational elements may be any sequence that is homologous with the target sequence in the genome d the host cell. Furthermore, the integrational elements may be non-encoding or encoding nucleic acid sequences. In order to promote targeted integration, the cloning vector is preferably linearized prior to transformation of the host cell. Linearization is preferably performed such that at least one but preferably either end of the cloning vector is flanked by sequences homologous to the target locus.

Preferably, the integrational elements in the expression vector, which are homologous to the target locus are derived from a highly expressed locus meaning that they are derived from a gene, which is capable of high expression level in the host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, the locus of SEQ ID NO:25, SEQ ID NO:26, SEQ ID NO:27, SEQ ID NO:28 and SEQ ID NO:29, preferably the A. niger locus of SEQ ID NO:25, SEQ ID NO:26, SEQ ID NO:27, SEQ ID NO:28 and SEQ ID NO:29 or a Trichoderma reesei cellobiohydrolase gene.

Alternatively, the vector may be integrated into the genome of the host cell by non-homologous recombination.

Alternatively and/or in combination with the embodiments described above, the DNA construct comprising the reporter gene in operative association with the promoter of the invention may be deleted from the host cell after the cell has been screened for production, preferably secretion, of a protein of interest in step (e) of the invention. Step (e) is described later. This enables the host cell to be used directly for the production of the protein of interest, without said host cell comprising a reporter gene and/or selectable marker gene. To enable deletion of the DNA construct of the invention, the DNA construct preferably comprises both the reporter gene and a bi-directional selectable marker gene between DNA repeats. The DNA repeats allow deletion of the DNA construct by intra chromosomal homologous recombination. This method is analogous to the “MARKER-GENE FREE” approach as described in EP 0 635 574 B1.

For autonomous replication, the vector may further comprise an origin of replication enabling the vector to replicate autonomously in the host cell in question. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75:1433).

More than one copy of the DNA construct of the present invention may be inserted into the host cell. This can be done, preferably by integrating into its genome copies of the DNA sequence, more preferably by targeting the integration of the DNA sequence at a highly expressed locus, preferably at a glucoamylase locus or at the locus of SEQ ID NO:25, SEQ ID NO:26, SEQ ID NO:27, SEQ ID NO:28 and SEQ ID NO:29. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase even more the number of copies of the DNA sequence to be over expressed the technique of gene conversion as described in WO98/46772 may be used.

The procedures used to ligate the elements described above to construct the recombinant expression vectors of the present invention are well known to one skilled in the art (see, e.g., Sambrook et al., 1989, supra).

### Host Cell

The present invention further relates to recombinant host cells transformed with the DNA construct defined in the former section comprising a promoter DNA sequence of the present invention in operative association with a reporter gene. Preferably, the transformed host cell further comprises an additional DNA construct comprising a DNA sequence comprising a coding sequence originating from a DNA library from an organism suspected of being capable of producing one or more proteins with properties of interest.

Such cells can be advantageously used in an expression cloning method as described in the next section. An expression vector comprising a promoter DNA sequence of the present invention in operative association with a reporter gene is introduced into a host cell. The host cell is further transformed with a DNA construct or expression vector comprising a DNA sequence comprising a coding sequence originating from a DNA library. Both DNA construct or expression vectors are either maintained as a chromosomal integrant or as a self-replicating extra-chromosomal vector as described earlier The choice of a host cell will to a large extent depend upon the organism wherein the DNA library has been made and/or upon the origin of the promoter DNA sequence used.

The host cell may be any microorganism. Preferably, the host cell is the same organism as the one wherein the DNA library has been prepared and/or as the one the promoter of the invention originates from as defined earlier in the specification. According to a preferred embodiment, the host cell is a prokaryote, or an eukaryote. According to a more preferred embodiment, the host cell useful in the methods of the present invention is a prokaryote, more preferably the prokaryote defined as source of promoter in the section promoter. More preferably, the prokaryotic host cell is a Bacillus host cell, preferably of the species Bacillus Subtilis, Bacillus licheniformis and/or Bacillus amyloliquefaciens.

According to another more preferred embodiment, the host cell useful in the methods of the present invention is a fungus.

The host cell may be a wild type filamentous fungus host cell or a variant, a mutant or a genetically modified filamentous fungus host cell. In a preferred embodiment of the invention the host cell is a protease deficient or protease minus strain. This may be the protease deficient strain Aspergillus oryzae JaL 125 having the alkaline protease gene named “alp” deleted (described in WO 97/35956 or EP 429 490), or the tripeptidyl-aminopeptidases (TPAP) deficient strain of A. niger, disclosed in WO 96/14404. Further, also host cell with reduced production of the transcriptional activator (prtT) as described in WO 01/68864 is contemplated according to the invention. Another specifically contemplated host strain is the Aspergillus oryzae BECh2, where the three TAKA amylase genes present in the parent strain IF04177 have been inactivated. In addition, two proteases, the alkaline protease and neutral metalloprotease 11 have been destroyed by gene disruption. The ability to form the metabolites cyclopiazonic acid and kojic acid has been destroyed by mutation. BECh2 is described in WO 00/39322 and is derived from JaL228 (described in WO 98/12300), which again was a mutant of IF04177 disclosed in U.S. Pat. No. 5,766,912 as A1560.

Optionally, the filamentous fungal host cell may comprise an elevated unfolded protein response (UPR) compared to the wild type cell to enhance production abilities of a polypeptide of interest. UPR may be increased by techniques described in US2004/0186070A1 and/or US2001/0034045A1 and/or WO01/72783A2 and/or WO2005/123763. More specifically, the protein level of HAC1 and/or IRE1 and/or PTC2 has been modulated, and/or the SEC61 protein has been engineered in order to obtain a host cell having an elevated UPR.

Alternatively, or in combination with an elevated UPR, the host cell may be genetically modified to obtain a phenotype displaying lower protease expression and/or protease secretion compared to the wild-type cell in order to enhance production abilities of a polypeptide of interest. Such phenotype may be obtained by deletion and/or modification and/or inactivation of a transcriptional regulator of expression of proteases. Such a transcriptional regulator is e.g. prtT. Lowering expression of proteases by modulation of prtT may be performed by techniques described in US2004/0191864A1 and in EP2005/055145.

Alternatively, or in combination with an elevated UPR and/or a phenotype displaying lower protease expression and/or protease secretion, the host cell may display an oxalate deficient phenotype in order to enhance the yield of production of a polypeptide of interest. An oxalate deficient phenotype may be obtained by techniques described in WO2004/070022A2 and/or WO2000/50576.

Alternatively, or in combination with an elevated UPR and/or a phenotype displaying lower protease expression and/or protease secretion and/or oxalate deficiency, the host cell may display a combination of phenotypic differences compared to the wild cell to enhance the yield of production of the polypeptide of interest. These differences may include, but are not limited to, lowered expression of glucoamylase and/or neutral alpha-amylase A and/or neutral alpha-amylase B, alpha-1, 6transglucosidase, protease, and oxalic acid hydrolase. Said phenotypic differences displayed by the host cell may be obtained by genetic modification according to the techniques described in US2004/0191864A1.

Alternatively, or in combination with phenotypes described here above, the efficiency of targeted integration of a nucleic acid construct into the genome of the host cell by homologous recombination, i.e. integration in a predetermined target locus, may preferably be increased by augmented homologous recombination abilities of the host cell. Such phenotype of the cell preferably involves a deficient hdfA or hdfB gene as described in WO2005/095624. WO2005/095624 discloses a preferred method to obtain a filamentous fungal cell comprising increased efficiency of targeted integration.

More preferably, the host cell of the invention is selected from the following list: a Bacillus, a yeast and a filamentous fungus, preferably an Aspergillus, Penicillium or Trichoderma species. Even more preferably, the Aspergillus host cell is an Aspergillus niger or Aspergillus sojae or Aspergillus oryzae species.

The present invention also relates to recombinant host cells, comprising more than one promoter DNA sequence of the present invention, each promoter being in operative association with a reporter gene. Such host cells may be advantageously used in an expression cloning method as described in the coming section. Bacterial cells may be transformed by a variety of methods including chemically induced competence (e.g. CaCl₂) or electroporation (Sambrook et al. (1989) “Molecular Cloning: a laboratory manual”, Cold Spring Harbor Laboratories, Cold Spring Harbor, N.Y.), protoplast transformation (Chang and Cohen (1979) Mol Gen Genet. 168: 111) and, in the case of some Bacillus species, natural competence (Spizizen (1958) Proc Natl Acad Sci USA 44: 1072). Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable procedures for transformation of Aspergillus and other filamentous fungal host cells using Agrobacterium tumefaciens are described in e.g. Nat. Biotechnol. 1998 September; 16(9):839-42. Erratum in: Nat Biotechnol 1998 November; 16(11):1074. Agrobacterium tumefaciens-mediated transformation of filamentous fungi. de Groot M J, Bundock P, Hooykaas P J, Beijersbergen A G. Unilever Research Laboratory Vlaardingen, The Netherlands. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920. Transformation of prokaryotes may be performed as described in EP 284 126A.

### Method of Expression Cloning

According to a further aspect, the present invention provides a method for isolating a DNA sequence comprising a DNA sequence coding for a protein of interest in a host cell, said method comprises the steps of:
(a) preparing a first DNA construct comprising a promoter DNA sequence operatively associated with a reporter gene conferring a selectable trait; said promoter DNA sequence being induced when the DNA construct is present in the host cell and when a protein of interest, preferably a secreted protein of interest, is produced by the host cell;
(b) preparing a second DNA construct comprising a DNA sequence comprising a DNA sequence coding for a protein of interest originating from a DNA library from an organism suspected of being capable of producing one or more proteins of interest;
(c) transforming a host cell with both DNA constructs prepared in (a) and (b);
(d) culturing all the transformed host cells obtained in (c) under conditions conducive to the production of the proteins of interest as present in the DNA library; and
(e) screening for transformed host cells producing a protein of interest by analysis of the proteins produced in (d).

### Step (a)

A first DNA construct is prepared, comprising a promoter DNA sequence operatively associated with a reporter gene conferring a selectable trait; said promoter DNA sequence being induced when the DNA construct is present in the host cell and when a protein of interest, preferably a secreted protein of interest, is produced by the host cell.

The promoter present in the first DNA construct is a promoter which is induced when a protein of interest, preferably a secreted protein of interest, is produced by a host cell.

In the present invention the term “induced” is defined as an increase in promoter activity of a promoter of the invention when a protein of interest is (over)produced, preferably secreted, by a host cell, compared to the activity of the promoter when the protein of interest is not (over)produced in a corresponding control host cell under the same culture conditions. Preferably, when a protein of interest is (over)produced, preferably secreted, in a host cell, promoter activity is at least about 1.5-fold increased, more preferably at least about two-fold, more preferably at least about three-fold, more preferably at least about four-fold, more preferably at least about five-fold, even more preferably at least about six-fold, even more preferably at least about eight-fold, even more preferably at least about ten-fold, even more preferably at least about twenty-fold, even more preferably at least about 50-fold and most preferably at least about 100-fold increased, compared to a corresponding control host cell wherein the protein of interest is not (over)produced under the same culture conditions. More preferably, the increase in promoter activity is infinite, i.e. no promoter activity is detected or the promoter is inactive when the protein of interest is not (over)produced, whereas the promoter is induced when the protein of interest is (over)produced. Promoter activity is preferably determined as defined previously. Most preferably, promoter activity is determined by mRNA analysis using Northern blot.

Such a promoter can be identified by comparing gene expression profiles of cells producing the protein of interest with that of a control strain not producing said protein. Preferably, such expression profiles are compared using well-established DNA micro-array analyses. Alternatively, expression profiles can be compared using other methods known to the skilled person such as Northern blot or quantitative PCR analysis. A most preferred method to compare expression profiles is performed by first comparing expression profiles by DNA micro array analyses, followed by subsequent corroboration of the micro array results by Northern Blot. According to a preferred embodiment, the promoter is the promoter DNA sequence of the invention described under the section promoter.

According to another preferred embodiment, when the host cell is a Bacillus cell, the promoter is derived from the following genomic DNA sequences as listed: htrA (SEQ ID NO:1) or htrB (SEQ ID NO:2). Preferably, the promoter DNA sequence is derived from the non coding part of one of these genomic DNA sequences situated upstream of the start codon or part of said genomic sequences situated upstream. More preferably, the promoter used is the DNA sequence of either SEQ ID NO:1 or SEQ ID NO:2.

According to another preferred embodiment, when the host cell is a filamentous fungal cell, the promoter is derived from the following genomic DNA sequences as listed: SEQ ID NO:20, SEQ NO: 21, SEQ ID NO: 22, SEQ ID NO: 23, or SEQ ID NO: 24. Preferably, the promoter DNA sequence is derived from the non coding part of one of these genomic DNA sequences situated upstream of the start codon or part of said genomic sequences situated upstream. More preferably, the promoter used is the DNA sequence, or derivative thereof, of either SEQ ID NO:15, SEQ NO: 16, SEQ ID NO: 17, SEQ ID NO: 18, or SEQ ID NO: 19.

The first DNA construct prepared in step (a) has already been described in the corresponding section “DNA construct” and preferably comprises a reporter gene conferring a selectable trait as described in the section “DNA construct”.

### Step, (b)

The DNA construct prepared in step (b) comprises a DNA sequence comprising a DNA sequence coding for a protein of interest originating from a DNA library from an organism suspected of being capable of producing one or more proteins of interest. The organism suspected of producing one or more proteins of interest usually is a prokaryote or an eukaryote. Examples of preferred prokaryotes and eukaryotes have already been defined earlier in the specification in connection with the origin of the promoter of the invention. According to one preferred embodiment, the organism is a Bacillus, more preferably a Bacillus species as defined in the section promoter. According to another preferred embodiment, the organism is an eukaryote, more preferably a fungus, of which most preferably a filamentous fungus.

The DNA construct prepared in step (b), preferably comprises regulatory sequences such as defined in the sections “DNA construct” and “Control elements”.

In the method according to the invention, the library of DNA fragments from an organism suspected of producing one or more proteins of interest can be a genomic library or a cDNA library. However, in the case of eukaryotic donors, preferably a cDNA library is used so as to avoid problems with recognition of promoters or splice signals in the fungal host organism. The cDNA library is preferably prepared from mRNA isolated from the source organism when grown under conditions conducive to the expression of the proteins of interest.

The method according to the invention can be applied to the isolation of DNA sequences coding for any protein or polypeptide of interest if there is an assay available for detection of the protein when expressed by the host cell.

### Step (c)

The identity of the host cell to be transformed with both DNA constructs has already been described under the section host cell. Methods of transformation have already been described in the same section. According to one embodiment, the host cell is simultaneously transformed with the constructs prepared in (a) and (b). Alternatively and according to another embodiment, the host cell is first transformed with the DNA construct prepared in step (a) and consecutively is transformed with the DNA construct prepared in step (b).

According to a preferred embodiment, the host cell is a prokaryote, preferably a Bacillus cell, more preferably a Bacillus subtilis, even more preferably a Bacillus subtilis deficient in the htrA gene, wherein at least part of the coding regions of the htrA gene have been deleted and/or replaced and/or wherein the non coding regions of the htrA gene are still present in the genome of said Bacillus cell, most preferably the Bacillus subtilis BV2003 is used (Hyyrylainen et al. (2001) Mol Microbiol 41: 1159).

Alternatively or in combination with former preferred embodiment, the host cell is a Bacillus cell and the reporter gene encodes a fluorescence reporter protein as defined in the section DNA construct, most preferably GFP.

Alternatively or in combination with former preferred embodiment, when the host cell is a Bacillus cell, the promoter is derived from the genomic DNA sequences as presented in the following list: htrA (SEQ ID NO:1) or htrB (SEQ ID NO:2). Preferably, the promoter sequence is derived from the non coding part of one of these two genomic DNA sequences situated upstream of the start codon or part of said sequence situated upstream. More preferably, the promoter used is one derived from either SEQ ID NO:1 or SEQ ID NO:2. According to a most preferred embodiment, the promoter used and the reporter gene used are either GFP in operative association with SEQ ID NO: 1 or GFP in operative association with SEQ ID NO: 2.

According to another preferred embodiment, the organism is an eukaryote, more preferably a fungus, of which most preferably a filamentous fungus. Preferably, the filamentous fungus is an Aspergillus, Penicillium or Trichoderma species. Even more preferably, the Aspergillus host cell is an Aspergillus niger or Aspergillus sojae or Aspergillus oryzae species. A most preferred host cell is A. niger, preferably CBS513.88 or derivative thereof.

Alternatively or in combination with former preferred embodiment, the host cell is a filamentous fungal cell and the reporter gene encodes a fluorescence reporter protein as defined in the section DNA construct, most preferably GFP. Alternatively, the reporter gene encodes a selectable marker. More preferably, the reporter gene is a hybrid reporter gene of a fluorescent protein and a selectable marker. Most preferably, the hybrid reporter gene is a comprises GFP and the phleomycin resistance gene (BLE) of Streptoalloteichus hindustanis (Drocourt et al., NAR, 1990).

Alternatively or in combination with the former preferred embodiment, when the host cell is a filamentous fungal cell, the promoter is derived from the genomic DNA sequences as presented in the following list: SEQ ID NO:20, SEQ ID NO:21, SEQ ID NO:22, SEQ ID NO:23 or SEQ ID NO:24. Preferably, the promoter sequence is derived from the non coding part of one of the listed genomic DNA sequences situated upstream of the start codon or part of said sequence situated upstream. More preferably, the promoter used is one of those used in examples 3 to 7 and derived from either SEQ ID NO:20, SEQ ID NO:21, SEQ ID NO:22, SEQ ID NO:23 or SEQ ID NO:24. According to a most preferred embodiment, the promoter used and the hybrid reporter gene used are either GFP-BLE (SEQ ID NO: 61) in operative association with promoter SEQ ID NO: 15, GFP-BLE in operative association with promoter SEQ ID NO: 16, GFP-BLE in operative association with promoter SEQ ID NO: 17, GFP-BLE in operative association with promoter SEQ ID NO: 18, or GFP-BLE in operative association with promoter SEQ ID NO: 19.

### Step (d)

After transformation of the host cells with both DNA constructs, all the obtained transformed host cells clones are cultured under conditions conducive to the production of the proteins of interest present in the DNA library. Depending on the assay required for detection of the protein of interest the transformed clones are propagated and stored as colonies on solid media such as agar plates or in liquid media, whereby the individual library clones are grown, stored and/or assayed in the wells of the microtiter plates.

The skilled person will understand that the usual adaptations to cloning methods known in the art can equally be applied to the method of the present invention. The adaptations include but are not limited to e.g. screening of pools of library clones, screening the same library for a number of different proteins of interest, as well as rescreening, reisolation and recloning of positive clones to ensure more accurate results.

A variety of methods are available to the skilled person for isolation of the DNA sequence encoding the protein of interest from the transformed host cell identified in the screening method, and for subsequent characterization of the isolated DNA sequence.

### Step (e)

Subsequent to step (d), the transformed host cells are screened for production, preferably secretion, of a protein of interest by analysis of the proteins produced. During step (e), one screens for transformed host cells producing, preferably secreting a protein of interest, by monitoring production-dependent, preferably secretion-dependent reporter expression induced via the promoter DNA sequence present in the first DNA construct.

According to a preferred embodiment, when the host cell is a Bacillus cell, screening to identify transformed host cells producing, preferably secreting, a protein of interest by monitoring production-dependent, preferably secretion-dependent, reporter expression induced by the promoter DNA sequence present in the first DNA construct, is preferably performed using fluorescent based cell analysis assays, e.g. fluorescent activated cell scanning, fluorescent activated cell sorting (FACS) or fluorimetric analysis. More preferably, the host cell is a Bacillus cell comprising a first DNA construct, said DNA construct comprising a promoter such as SEQ ID NO: 1 or derivative thereof or SEQ ID NO: 2 or derivative thereof, said DNA construct further comprising a reporter gene encoding a fluorescent reporter and screening is performed using fluorescence based cell analysis assays. Even more preferably, the host cell is a Bacillus cell and the first DNA construct comprises:
the promoter DNA sequence SEQ ID NO: 1 or derative thereof, in operative association with GFP, or
the promoter DNA sequence SEQ ID NO: 2 or derative thereof, in operative association with GFP

and screening is performed using FACS. Alternatively, or in combination with the former embodiment, screening may be performed using e.g. colorimetric assays or using selective culture conditions. The skilled person will understand that depending on the reporter gene used, the selective culture conditions should be chosen accordingly. Examples of selective culture conditions are, but are not limited to, the use of biocides, the use of antibiotics, limitation of at least one nutrient, prototrophy.

According to another preferred embodiment, when the host cell is a filamentous fungal cell, screening to identify transformed host cells producing, preferably secreting, a protein of interest by monitoring production-dependent, preferably secretion-dependent, reporter expression induced by the promoter DNA sequence present in the first DNA construct of the invention, is preferably performed by cultivation under selective culture conditions. The skilled person will understand that depending on the reporter gene used, the selective culture conditions should be chosen accordingly. Examples of selective culture conditions are, but are not limited to, the use of biocides, the use of antibiotics, limitation of at least one nutrient, prototrophy. Alternatively, or in combination with the former embodiment, screening may be performed using e.g. colorimetric, fluorimetric (like FACS) or enzyme activity based assays. According to a more preferred embodiment, the host cell is a filamentous fungal cell comprising a first DNA construct of the invention and screening is performed using selective culture conditions. More preferably, the host cell is a filamentous fungal cell comprising a first DNA construct of the invention, said DNA construct comprising a promoter selected from the list of: SEQ ID NO:15 or derivative thereof, SEQ ID NO:16 or derivative thereof, SEQ ID NO:17 or derivative thereof, SEQ ID NO:18 or derivative thereof, SEQ ID NO:19 or derivative thereof, said DNA construct further comprising a reporter gene encoding a selectable marker and screening is performed using selective culture conditions. Even more preferably, the host cell is a filamentous fungal cell comprising a first DNA construct of the invention, said DNA construct comprising a promoter selected from the list of: SEQ ID NO:15 or derivative thereof, SEQ ID NO:16 or derivative thereof, SEQ ID NO:17 or derivative thereof, SEQ ID NO:18 or derivative thereof, SEQ ID NO:19 or derivative thereof, said DNA construct further comprising a hybrid reporter gene such as SEQ ID NO: 61 and screening is performed using selective culture conditions.

According to another more preferred embodiment, the host cell is an A. niger cell comprising a first DNA construct of the invention and screening is performed using selective culture conditions. More preferably, the host cell is an A. niger cell comprising a first DNA construct of the invention, said DNA construct comprising a promoter selected from the list of: SEQ ID NO:15 or derivative thereof, SEQ ID NO:16 or derivative thereof, SEQ ID NO:17 or derivative thereof, SEQ ID NO:18 or derivative thereof, SEQ ID NO:19 or derivative thereof, said DNA construct further comprising a reporter gene encoding a selectable marker and screening is performed using selective culture conditions. Even more preferably, the host cell is an A. niger cell comprising a first DNA construct of the invention, said DNA construct comprising a promoter selected from the list of: SEQ ID NO:15 or derivative thereof, SEQ ID NO:16 or derivative thereof, SEQ ID NO:17 or derivative thereof, SEQ ID NO:18 or derivative thereof, SEQ ID NO:19 or derivative thereof, said DNA construct further comprising a hybrid reporter gene such as SEQ ID NO:61 and screening is performed using selective culture conditions. Even more preferably, the host cell is an A. niger cell and the first DNA construct comprises:
hybrid reporter gene GFP-BLE (SEQ ID NO: 61) in operative association with promoter SEQ ID NO: 15, or
GFP-BLE in operative association with promoter SEQ ID NO: 16, or derivative thereof, or
GFP-BLE in operative association with promoter SEQ ID NO: 17, or derivative thereof, or
GFP-BLE in operative association with promoter SEQ ID NO: 18, or derivative thereof, or
GFP-BLE in operative association with promoter SEQ ID NO: 19, or derivative thereof, and screening is performed using selective culture conditions comprising an antibiotic, preferably phleomycin as selective agent.

The DNA sequences isolated by the screening method of the invention as described above are used to produce, or to improve the production of, a protein of interest encoded by the DNA sequence. Advantageously, the transformed host cell as isolated in the above described screening method is used directly in a process for the production of the protein of interest by culturing the transformed host cell under conditions conducive to the production, preferably secretion of the protein of interest and, optionally, recovering the protein.

According to a preferred embodiment, the host cell is enabled to be used directly for the production of the protein of interest, without said host cell comprising a reporter gene and/or selectable marker gene. The DNA construct comprising the reporter gene in operative association with the promoter of the invention is therefore deleted from the host cell after the cell has been screened for production, preferably secretion, of a protein of interest. To enable deletion of the DNA construct of the invention, the DNA construct preferably comprises both the reporter gene and a bi-directional selectable marker gene between DNA repeats. The DNA repeats allow deletion of the DNA construct by intra chromosomal homologous recombination. This method is analogous to the “MARKER-GENE FREE” approach as described in EP 0 635 574 B1.

Often the initial transformed host cell isolated in the screening method of the invention will have an expression level which is satisfactory for screening purposes but which can be significantly improved for economic production purposes. To this end, the DNA sequence is inserted into an expression vector which is subsequently used to transform a suitable host cell. In the expression vector the DNA sequence is operably linked to appropriate expression signals, such as a promoter, optionally a signal sequence and a terminator, which are capable of directing the expression of the protein in the host organism as defined in the sections “DNA construct” and “control elements”. A suitable host cell for the production of the protein may be either a prokaryotic or eukaryotic cell, preferably a eubacterium, yeast or filamentous fungus. Preferred bacterial host cells are selected from the genus Bacillus as defined in the section promoter. According to another preferred embodiment, the host cell is a yeast or a filamentous fungus as already defined in the section “host cell”. Preferred yeast host cells are selected from the group consisting the genera Saccharomyces, Kluyveromyces, Yarrowia, Pichia, and Hansenula. Preferred filamentous fungal host cells are selected from the same genera listed above as preferred host cells for the screening method. More preferably, the filamentous fungus is an Aspergillus, Penicillium or Trichoderma species. Even more preferably, the Aspergillus host cell is an Aspergillus niger or Aspergillus sojae or Aspergillus oryzae species. A most preferred host cell is A. niger, preferably CBS513.88 or derivative thereof.

The suitable host cell is transformed with the expression vector by any of the various protocols available to the skilled person. The transformed host cell is subsequently used in a process for producing the protein of interest by culturing the transformed host cell under conditions conducive to the expression of the DNA sequence encoding the protein, and optionally recovering the protein.

Preferably, the protein of interest is an enzyme.

Accordingly, the invention relates to a use of the promoter of the invention in an expression cloning method.

The invention further relates to other use of the promoter of the invention. Preferably, the promoter of the invention may be operatively associated with any coding sequence in a DNA construct or expression vector all defined in former sections. Such DNA contruct or expression vector may be introduced into any host cell as defined in “host cell” section and used for expression of a given valuable compound. Examples of such valuable compound are, but are not limited to, a metabolite or a polypeptide. Preferably, the coding sequence operatively associated with the promoter of the invention encodes the valuable compound. Alternatively, and according to another preferred embodiment of the invention, the coding sequence operatively associated with the promoter of the invention is involved in the production of the valuable compound.

Alternatively and according to another preferred use of the promoter of the invention, the promoter of the present invention is incorporated into an inactivation or replacement construct, transformed into a given host cell and used to inactivate or replace a target gene.

The present invention is further illustrated by the following examples.

### EXAMPLES

### Materials and Methods

### General Procedures

Standard molecular cloning techniques such as DNA isolation, gel electrophoresis, enzymatic restriction modifications of nucleic acids, Northern analyses, E. coli transformation, etc, were performed as described by Sambrook et al. (2001) “Molecular Cloning: a laboratory manual, 3^{rd} edition”, Cold Spring Harbor Laboratories, Cold Spring Harbor, N.Y. and Innis et al. (1990) “PCR protocols, a guide to methods and applications” Academic Press, San Diego. Synthetic oligo deoxynucleotides were obtained from Invitrogen (Breda, The Netherlands). Room temperature is 20 degrees Celsius±2 degrees Celsius.

Transformation of Aspergillus niger.

Transformation of A. niger was performed according to the method described by Tilburn, J. et al. (1983) Gene 26, 205-221 and Kelly, J. & Hynes, M. (1985) EMBO J., 4, 475-479 with the following modifications:

Spores were germinated and cultivated for 16 hours at 30 degrees Celsius in a shake flask placed in a rotary shaker at 300 rpm in Aspergillus minimal medium (100 ml). Aspergillus minimal medium contains per litre: 6 g NaNO₃, 0.52 g KCl, 1.52 g KH₂PO₄, 1.12 ml 4 M KOH, 0.52 g MgSO₄.7H₂O, 10 g glucose, 1 g casaminoacids, 22 mg ZnSO₄.7H₂O, 11 mg H₃BO₃, 5 mg FeSO₄.7H₂O, 1.7 mg CoCl₂.6H₂O, 1.6 mg CuSO₄.5H₂O, 5 mg MnCl₂.2H₂O, 1.5 mg Na₂MoO₄.2H₂O, 50 mg EDTA, 2 mg riboflavin, 2 mg thiamine-HCl, 2 mg nicotinamide, 1 mg pyridoxine-HCL, 0.2 mg panthotenic acid, 4 g biotin, 10 ml Penicillin (5000 IU/ml) Streptomycin (5000 UG/ml) solution (Gibco).
Novozym 234™ (Novo Industries) instead of helicase was used for the preparation of protoplasts;
after protoplast formation (60-90 minutes), KC buffer (0.8 M KCl, 9.5 mM citric acid, pH 6.2) was added to a final volume of 45 ml, the protoplast suspension was centrifuged for 160 minutes at 3000 rpm at 4 degrees Celsius in a swinging-bucket rotor. The protoplasts were resuspended in 20 ml KC buffer and subsequently 25 ml of STC buffer (1.2 M sorbitol, 10 mM Tris-HCl pH 7.5, 50 mM CaCl₂) was added. The protoplast suspension was centrifuged for 10 minutes at 3000 rpm at 4 degrees Celsius in a swinging-bucket rotor, washed in STC-buffer and resuspended in STC-buffer at a concentration of 10E8 protoplasts/ml;
to 200 microliter of the protoplast suspension, the DNA fragment, dissolved in 10 microliter TE buffer (10 mM Tris-HCl pH 7.5, 0.1 mM EDTA) and 100 microliter of PEG solution (20% PEG 4000 (Merck), 0.8 M sorbitol, 10 mM Tris-HCl pH 7.5, 50 mM CaCl₂) was added;
after incubation of the DNA-protoplast suspension for 10 minutes at room temperature, 1.5 ml PEG solution (60% PEG 4000 (Merck), 10 mM Tris-HCl pH 7.5, 50 mM CaCl₂) was added slowly, with repeated mixing of the tubes. After incubation for 20 minutes at room temperature, suspensions were diluted with 5 ml 1.2 M sorbitol, mixed by inversion and centrifuged for 10 minutes at 4000 rpm at room temperature. The protoplasts were resuspended gently in 1 ml 1.2 M sorbitol and plated onto solid selective regeneration medium consisting of either Aspergillus minimal medium without riboflavin, thiamine.HCL, nicotinamide, pyridoxine, panthotenic acid, biotin, casaminoacids and glucose. In case of acetamide selection the medium contained 10 mM acetamide as the sole nitrogen source and 1 M sucrose as osmoticum and C-source. Alternatively, protoplasts were plated onto PDA (Potato Dextrose Agar, Oxoid) supplemented with 1-50 microgram/ml phleomycin and 1 M sucrose as osmosticum. Regeneration plates were solidified using 2% agar (agar No. 1, Oxoid L11). After incubation for 6-10 days at 30 degrees Celsius, conidiospores of transformants were transferred to plates consisting of Aspergillus selective medium (minimal medium containing acetamide as sole nitrogen source in the case of acetamide selection or PDA supplemented with 1-50 microgram/ml phleomycin in the case of phleomycin selection) with 2% glucose and 1.5% agarose (Invitrogen) and incubated for 5-10 days at 30 degrees Celsius. Single transformants were isolated and this selective purification step was repeated once upon which purified transformants were stored.
   Aspergillus niger Microtiterplate Fermentation and Sampling

To obtain spores of A. niger, mycelium was transferred to microtiterplates filled with 150 microliter solid PDA medium (Potato Dextrose Agar, Oxoid), prepared according to the supplier's instructions. After growth for 3-7 days at 30 degrees Celsius (static) spores had formed. Subsequently 100 microliter liquid fermentation medium (70 g/l glucose.H2O, 25 g/l peptone (from casein), 12.5 g/l yeast extract (Difco), 2 g/l K₂SO₄, 1 g/l KH₂PO₄, 0.5 g/l MgSO₄.7H₂O, 0.03 g/l ZnCL₂, 0.02 g/l CaCL₂, 9 mg/l MnSO₄.H₂O, 3 mg/l FeSO₄.7H₂O, adjusted to pH 5.6 with 4 NH₂SO₄), was added to each well. The fermentation medium was pipetted up and down 20 times to isolate the spores in the medium. The inoculated fermentation medium was transferred to another microtiterplate and incubated at 34 degrees Celsius, 550 rpm and 80% humidity for 6 days in an orbital microtiterplate shaker (Infors HT). On day 6 supernatants were collected and used for detection of secreted proteins.

### Mass Spectrometry Analysis

Supernatants were ultra-filtrated by centrifugal force on an Ultracel filtration plate (Millipore, Billerica, Mass., USA). Ultra-filtration was performed according to the protocol provided by the manufacturer. To the ultra-filtrated supernatants 10 microliter 200 mM ammoniumbicarbonate, pH 7,8 (Riedel-de Haen A G, Seelze, Germany) and 1 microliter 250 microgram per ml trypsin (Worthington, Lakewood, N.J., USA) were added. The supernatants were digested for 3 hours at 37 degrees Celsius. The digested supernatants were mixed in a 1:1 ratio with 10 milligram per ml recrystallized alpha-cyano-4-hydroxycinnamic acid (?CHCA) (Laser Bio Labs, Sophia-Antipolis Cedex, France) solution; from these mixtures 1 microliter was spotted on the MALDI target. The spotted samples were analyzed with MALDI MS and MALDI MS/MS on a vMALDI LTQ mass spectrometer (Thermo electron, Orlando, Fla., USA).

The obtained MS/MS spectra, converted to peptide sequences, were compared to available protein sequences in pre-constructed FASTA protein databases, to identify individual enzymes.

### RNA Isolation

Aspergillus niger mycelium formed on 20 ml liquid selective regeneration medium (6 g/l NaNO₃, 0.52 g/l KCl, 1.52 g/l KH₂PO₄, 0.25 g/l KOH, 0.52 g/l MgSO₄.7H₂O, 22 mg/l ZnSO₄.7H₂O, 11 mg/l H₃BO₃, 5 mg/l FeSO₄.7H₂O, 1.7 mg/l CoCl₂.6H₂O, 1.6 mg/l CuSO₄.5H₂O, 5 mg/l MnCl₂.2H₂O, 1.5 mg/l Na₂MoO₄.2H₂O, 50 mg/l EDTA, 341 g/l sucrose, 10 ml Penicillin (5000 IU/ml) Streptocmycin (5000 UG/ml) solution (Gibco)) in a petridish was harvested, washed with demineralized water and squeezed between paper towels to remove excessive water. Mycelium was frozen immediately in liquid nitrogen and grinded to a fine powder using mortar and pestle. The resulting powder was transferred to a sterile 50 ml tube and weighed upon which for every 1-1.2 g of ground mycelium 10 ml TRIzol reagent (Invitrogen) was added (max. 25 ml per tube). The mycelial powder was immediately solubilized by vigorous mixing (vortexing, 1 min.), followed by 5 min room temperature incubation with occasional mixing. 0.2 (original TRIzol) volumes of chloroform (thus 2 ml for every 10 ml TRIzol used originally) was added, vortexed and left at room temperature for 10 min. Subsequently, the mixture was centrifuged at 4 degrees Celsius, 6000 g for 30 minutes. The top aqueous phase was transferred to a fresh tube and total RNA was precipitated by addition of 0.5 (original TRIzol) volumes of 100% isopropyl alcohol (thus 5 ml of isopropyl alcohol for every 10 ml TRIzol used originally). After 10 minutes precipitation at room temperature, the RNA was recovered by centrifugation for 30 minutes at 6000 g. Upon removal of supernatant, the RNA pellet was rinsed with one (original TRIzol) volume of 70% ethanol. After removal of the ethanol, the RNA pellet was air dried. The dried RNA pellet was dissolved in 3 ml GTS (100 mM Tris-Cl, pH 7.5, 4 M guanidium thiocyanate, 0.5% sodium lauryl sarcosinate) buffer. Additional clean-up of the RNA was performed using Rneasy Maxi Kit (Qiagen) according to suppliers manual. 10 microliter of RNA solution was used to determine quality and concentration of nucleic acids.

Aspergillus niger Strains

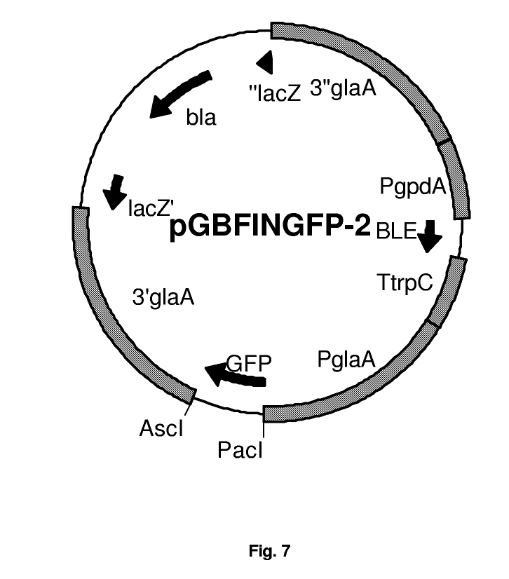

A. niger WT-1: This A. niger strain is CBS513.88 comprising deletions of the genes encoding glucoamylase (glaA), fungal amylase and acid amylase. A. niger WT 1 was constructed by using the “MARKER-GENE FREE” approach as described in EP 0 635 574 B1. In this patent it is extensively described how to delete glaA specific DNA sequences in the genome of CBS 513.88. The first procedure resulted in a MARKER-GENE FREE ?glaA recombinant A. niger CBS 513.88 strain, possessing finally no foreign DNA sequences at all. Subsequently, the genes encoding fungal amylase and acid amylase were deleted from the genome of A. niger WT-1 by the method described in EP 0 635 574 B1. Vectors used for cloning of the desired constructs as depicted in FIGS. 7 to 9 and 11 to 14 contain backbone features previously described in WO 99/32617.

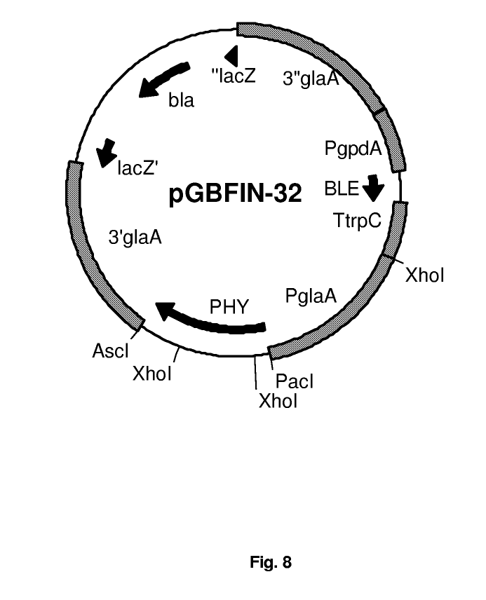

A. niger WT-GFP: This A. niger strain was obtained by transformation of A. niger WT-1 with the expression vector pGBFINGFP-2 depicted in FIG. 7 containing the gene coding for the well-known Green Fluorescent Protein (GFP))(Chalfie, M et al., Science (1994) 263(5148): 802-805) driven by the glucoamylase promoter. After purification on selective medium, a single copy integrant was selected based on PCR analysis.
A. niger WT-PHY: This A. niger strain was obtained by transformation of the A. niger WT-1 with the expression vector pGBFIN-32 depicted in FIG. 8 containing the phytase gene PHY (identical to FytA previously described in WO 99/32617) driven by the glucoamylase promoter. After purification on selective medium, a single copy integrant was selected based on PCR analysis.

A. niger WT-PHY-2: This A. niger strain was obtained by transformation of the A. niger WT-1 with the expression vector pGBFIN-32 depicted in FIG. 8 containing the phytase gene PHY (identical to FytA previously described in WO 99/32617) driven by the glucoamylase promoter. After purification on selective medium, a multy copy integrant was selected based on PCR analysis.

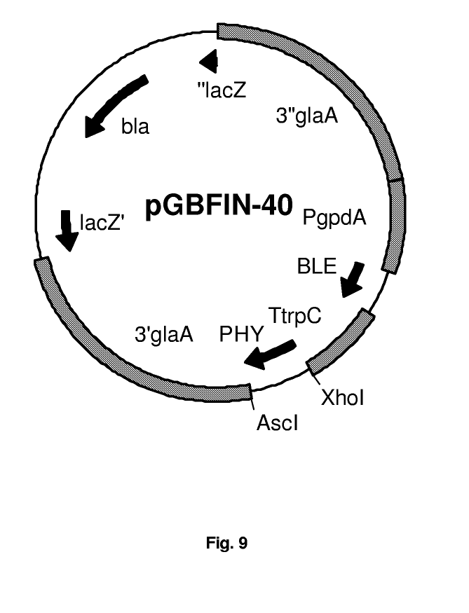

A. niger WT-vector: This A. niger strain was obtained by transformation of the A. niger WT-1 strain with empty control vector pGBFIN-40, depicted in FIG. 9. The empty control vector pGBFIN-40 was constructed by XhoI digestion of pGBFIN-32 depicted in FIG. 8, and re-ligation of the largest fragment, thereby removing the glucoamylase promoter and the 5′ part of phytase gene PHY. A single copy integrant was selected by PCR analysis.

### Example 1

### Construction of Bacillus Reporter Plasmids and Reporter Strains

1.1 Construction of Reporter Plasmids pPhtrA-gfp-amyE and pPhtrB-gfp-amyE

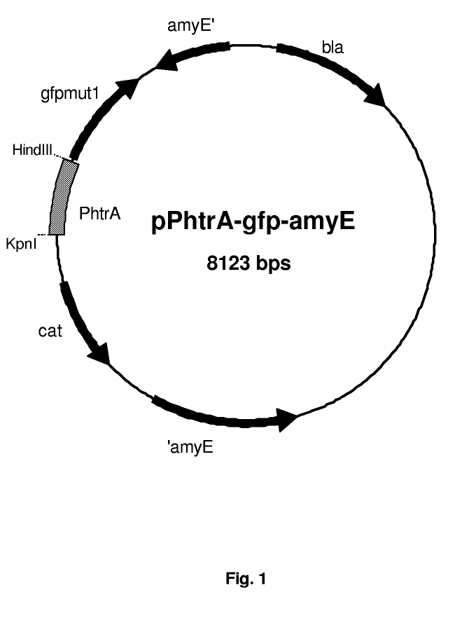

First, reporter plasmid pPhtrA-gfp-amyE was constructed as follows. The gene encoding the optimized version of GFP, gfpmut-1 (P. Cormack, R. H. Valdivia, and S. Falkow, 1996; FACS-optimized mutants of the green fluorescent protein (GFP), Gene 173, 33.), was amplified by PCR, using pSG1151 (P. J. Lewis and A. L. Marston, 1999; GFP vectors for controlled expression and dual labelling of protein fusions in Bacillus subtilis, Gene 227, 101.) as template DNA, with the following primers: RN-lacZ-rv (SEQ ID NO: 3), GTGAGCGGATGCAATTTCACACAGG; and gfp-terminator-rv, (SEQ ID NO: 4) GTGGCTCAGCTTTTTTAAGGAAGGGAGGCTCTCACCTCCCTTCCCTTTATTTGTAGAGC TCATCCATGCC, resulting in KpnI and Bpu1101I (in bold) sites up- and downstream gfpmut-1, respectively. The 913 bp PCR product was ligated in pDL (containing homologous flanking regions of the amyE locus for site specific recombination, (G. Yuan and S. L. Wong, 1995; Regulation of groE expression in Bacillus subtilis: the involvement of the sigma A-like promoter and the roles of the inverted repeat sequence (CIRCE), J. Bacteriol. 177, 5427), using the KpnI and Bpu11011 sites, resulting in pGFP-amyE. The promoter of htrA (P_{htrA}) (SEQ ID NO: 1) was amplified by PCR using chromosomal DNA of B. subtilis 168 (Bacillus Genetic Stock Center ID: 1A1) as template DNA. Primers used were: PhtrA-fw, (SEQ ID NO: 5) CGTGAGGTACCGGCTTCTGTTTCTGCC; and Phtr-rv, (SEQ ID NO: 6) CATCACGAAGCTTATCCATCATGTTCACTCCG, introducing Kpn1 and HindIII sites (in bold), respectively. After digestion with Kpn1 and Hind3, the 531 bp PCR product was ligated into pGFP-amyE, upstream the gfp gene resulting in reporter plasmid pPhtrA-gfp-amyE. A map of this plasmid is depicted in FIG. 1. Plasmid pPhtrB-gfp-AmyE was constructed in a similar way as described above. The primers used for the amplification of the promoter of htrB (P_{htrB}) (SEQ ID NO 2) were PhtrB-fw (SEQ ID NO 7) GACCGGTACCTCAGGATCTTTCGCC and PhtrA-rv (SEQ ID NO 8) GGCCATCAAGCTTATAATCCATGTTCTTACACTCC.

### 1.2 Construction of the Bacillus Reporter Strains VT210A and VT210B

The resulting plasmids pPhtrA-gfp-amyE and pPhtrB-gfp-amyE were introduced into B. subtilis DB104, a DaprE, DnprE double mutant (F. Kawamura and R. H. Doi, 1984; Construction of a Bacillus subtilis double mutant deficient in extra cellular alkaline and neutral proteases, J. Bacteriol. 160, 442). Chloramphenicol resistant transformants were checked for site-specific integration by double crossover at the amyE locus by PCR on chromosomal DNA. To reduce negative feedback regulation by HtrA of P_{htA}, (D. Noone, A. Howell, R. Collery, and K. M. Devine, 2001; YkdA and YvtA, HtrA-like serine proteases in Bacillus subtilis, engage in negative auto regulation and reciprocal cross-regulation of ykdA and yvtA gene expression, J. Bacteriol. 183, 654) the new strain was transformed with chromosomal DNA of B. subtilis BV2003, which has a disrupted htrA gene by integration of pMutin2 (H. L. Hyyrylainen, A. Bolhuis, E. Darmon, L. Muukkonen, P. Koski, M. Vitikainen, M. Sarvas, Z. Pragai, S. Bron, J. M. van Dijl, and V. P. Kontinen, 2001; A novel two-component regulatory system in Bacillus subtilis for the survival of severe secretion stress, Mol. Microbiol. 41, 1159). Transformants were selected for erythromycin resistance and checked by PCR on chromosomal DNA for htrA disruption. The obtained strains were designated VT200A and VT200B.

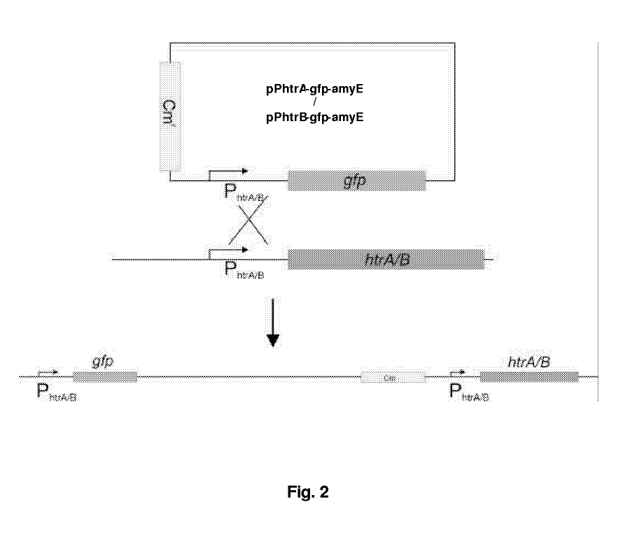

In order to increase transformation efficiency of the VT200A strain, a BsuMR disruption construct was designed. The BsuMR system is formed by an operon consisting of three genes, ydiR, ydiS and ydjA, of which each is essential for BsuMR functioning (P. J. Lewis and A. L. Marston, 1999; GFP vectors for controlled expression and dual labeling of protein fusions in Bacillus subtilis, Gene 227,101). Flanking region 1 (flr1), a 756 bp region of ydiR was amplified using the following primers: flrBsu1F, (SEQ ID NO 9) GAAAGATTGTTTCAGAAGCC; and frl1 BsuR, (SEQ ID NO 10) ACAGCGTTGGGATCCAAGCCCTTCCATTTTGGACATTTGG and chromosomal DNA of B. subtilis as a template. The spectinomycin resistance marker was amplified using pDG1726 (A. M. Guerout-Fleury, K. Shazand, N. Frandsen, and P. Stragier, 1995; Antibiotic-resistance cassettes for Bacillus subtilis, Gene 167, 335) as template DNA and with the following primers: specBsu-F, (SEQ ID NO 11) GGGCTTGGATCCCAACGCTGTCGACGTTGTAAAACGACGG; and specBsu-R, (SEQ ID NO 12) CGCATAGCTTTCCGGTCGCCGCAGCTATGACCATGATTACGC. The 1319 bp PCR product and the flrBsu1 fragment were used in a second PCR in which both fragments served as one template upon recombination of their homologous ends introduced by primers flrBsu1-R and specBsu-F (in bold). Using primers flrBsu1-F and specBsu-R, a 2075 bp PCR fragment, flr1-spec^{r}, was obtained, which was sub cloned in pCR-XL-TOPO (Invitrogen) resulting in pCT-XL-TOPO-flr1-spec. Flanking region 2, a 739 bp region covering 24 bp of the 3′ end of ydiS (encoding a DNA endonuclease) and a large part of the adjacent ydjA (unknown function), was amplified using primer flrBsu2-F, (SEQ ID NO 13) GATGATTCGTCTTTTTGTAGTG; and primer flrBsu2-R, (SEQ ID NO 14) CGATCAACATATGTGTTCACG and chromosomal DNA of B. subtilis as a template. The PCR product was cloned in pCR-XL-TOPO resulting in pCR-XL-TOPO-flr2. Making use of the multiple cloning site of the pCR-XL-TOPO vector, flr1-spec^{r} was restricted from pCR-XL-TOPO-flr1 using XhoI and NsiI and ligated into pCR-XL-TOPO-flr2, digested with XhoI and PstI (compatible to NsiI). This resulted in the deletion construct pTOPO-flr1-spec^{r}-flr2, which was introduced into strain VT200A. Spectinomycin resistant transformants were checked for the ydiS deletion by PCR on chromosomal DNA. The obtained, final reporter strains were designated VT210A and VT210B and used for further experiments. A general outline of the construction of these strains is depicted in FIG. 2.

1.3 Construction of Expression Vectors pUBnpr2 and pUBBAG:

The construction of plasmid pUB110 and its sequence was described before (McKenzie T., Hoshino T., and Sueoka, N. 1986. The nucleotide sequence of pUB110; some salient features in relation to replication and its regulation. Plasmid 15: 93-103). Cloning of the Bacillus amyloliquefaciens a-amylase gene amyQ (Genbank accession number J 01542) in pUB110 lead to plasmid pKTH10 as described before (Palva, I. 1982. Molecular cloning of the alpha-amylase gene of Bacillus amyloliquefaciens and its expression in B. subtilis. Gene 19: 81-87).

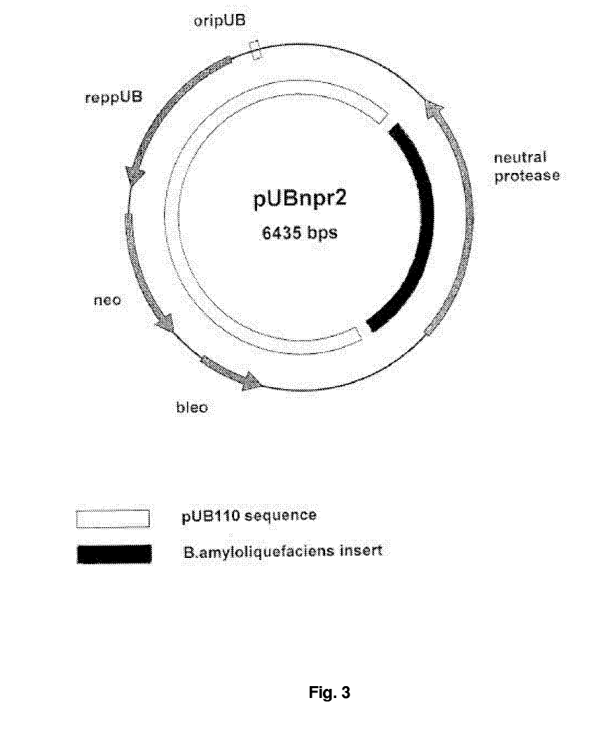

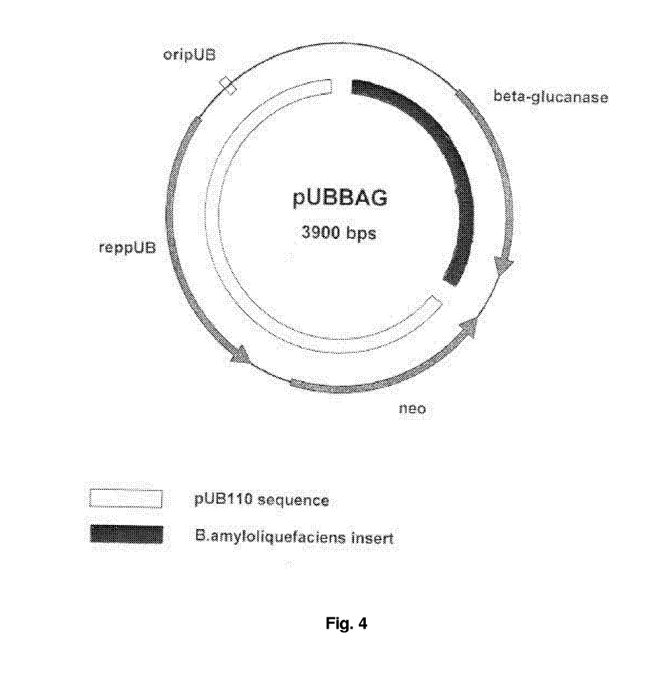

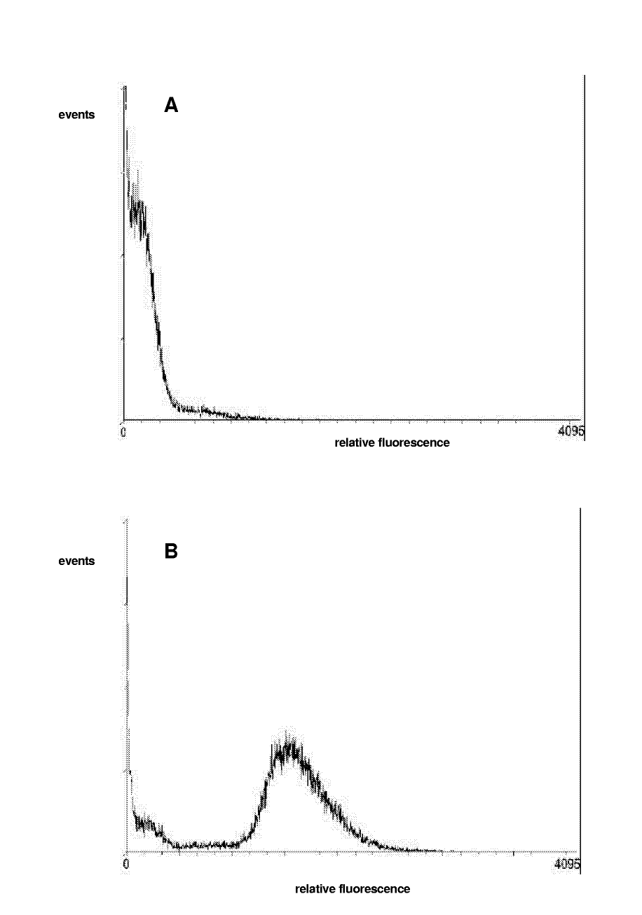

Cloning of the neutral protease gene npr (Genbank accession number K 02497) and β-glucanase gene bag (Genbank accession number M15674) both from Bacillus amyloliquefaciens into vector pUB110 was performed essentially according to the method used for the cloning of amyQ, resulting in plasmids pUBnpr2 and pUBBAG as depicted in FIGS. 3 and 4.

1.4 Transformation of Expression Constructs pKTH10, pUBnpr2 and pUBBAG into Bacillus Reporter Strain VT210A:

Transformation of Bacillus subtilis strain VT210A was essentially performed as described before making use of the natural competence of this organism (Anagnostopoulos, C. and Spizizen, J. 1961. Requirements for transformation in Bacillus subtilis. J. Bacteriology 81: 741-746). After DNA incubation the cells were spread on 2×TY agar containing 0.1% starch and the antibiotic kanamycin (20 μg/ml) as a selective agent for transformation. Plates were incubated over night at 37° C. When plasmid pKTH10 was involved in transformation the colonies were covered with a lugol solution to detect the amylase secreting cells.

### Example 2

### Analysis of Secretion Stressed Cells by Fluorescent Activated Cell Assays

### 2.1 Selection of Secretion Stressed Cells by Fluorescence Activated Cell Sorting:

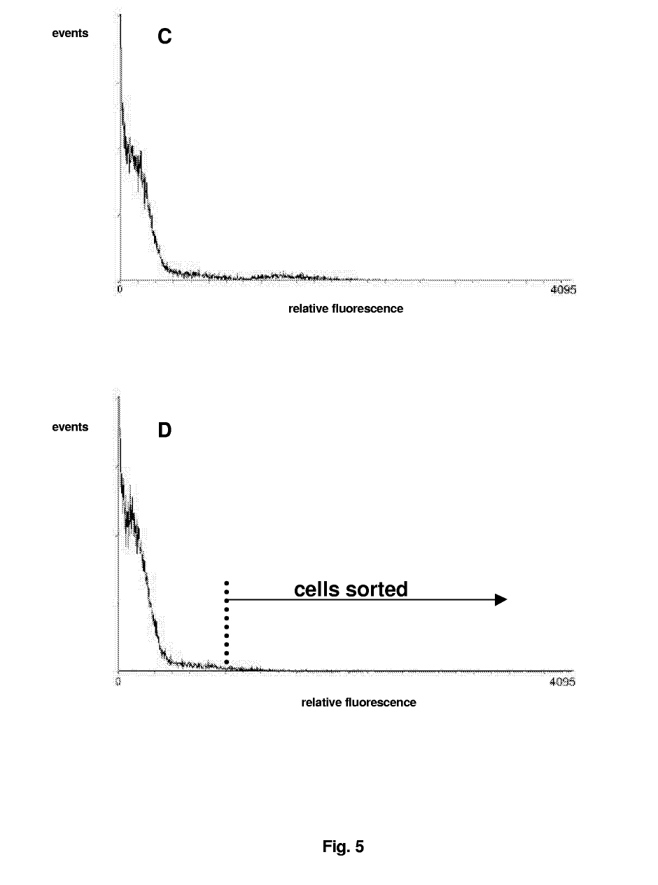

The plasmids pUB110 (empty control vector) and pKTH10 (encoding Bacillus amyloliquefaciens a-amylase gene amyQ) were used in a ratio of 1:20 for co-transformation of Bacillus subtilis strain VT210A. One part of the cells was spread on selective TY agar plates, and to the other part 2×TY broth containing kanamycin, was added and the cells were cultivated over night. The separate plasmids were transformed as well, performing the same operations as for the co-transformation. The next day, cultures were analyzed for GFP fluorescence on a flow cytometer type FACS (Mo-Flo of Dako-cytomation) by a skilled operator. A blue laser of 80 mWatt, and 488 nm was used. A typical FACS experiment comprised analysis of 20.000 events (cells). First, fluorescence signals of cells of VT210A, pUB110 (no secretion stress) and of VT210A, pKTH10 (secretion stress due to overproduction of the alpha-amylase AmyQ) were analyzed separately (FIGS. 5A and 5B) and as a mix (FIG. 5C). On basis of these results a cell-sorting limit was manually set (dashed line in FIG. 5D), such that cells demonstrating GFP fluorescence higher than the cell-sorting limit, were sorted. The co-transformation culture was applied to the flow cytometer and sorted cells were collected in a tube. To verify that the sorted fluorescent population had been enriched for alpha-amylase secreting cells, different dilutions of the collected cells were spread on selective agar containing starch. The same was done with the original overnight input culture, which was not sorted by FACS. After growth, the colonies were covered by a lugol solution to visualize halo formation caused by alpha-amylase activity. As shown in table 1, about 90% of the sorted cells indeed secreted alpha-amylase, whereas in the input culture only 0.5-0.7% of the cells secreted alpha-amylase. This result clearly demonstrates the powerful selection tool of the present invention by isolation of protein secreting cells using discrimination in fluorescent signal between normal cells and cells stressed by secretion.

TABLE 1
enrichment for amy+ colony forming units after flow cytometry.

| | Number | Number | | |
|---|---|---|---|---|
| Used cells | of cfu | of amy⁺ | Dilution | % amy⁺ |
| Pre-FACS of over night | >2000 | 14 | 10⁻⁴ | <0.7 |
| grown co-transformation | 600 | 3 | 10⁻⁵ | 0.5 |
| Sorted cells of over night | 111 | 104 | 10⁻¹ | 93.7 |
| grown co- transformation | 9 | 8 | 10⁻² | 88.9 |

### 2.2 Selection of Secretion Stressed Cells by Fluorescent Cell Scanning:

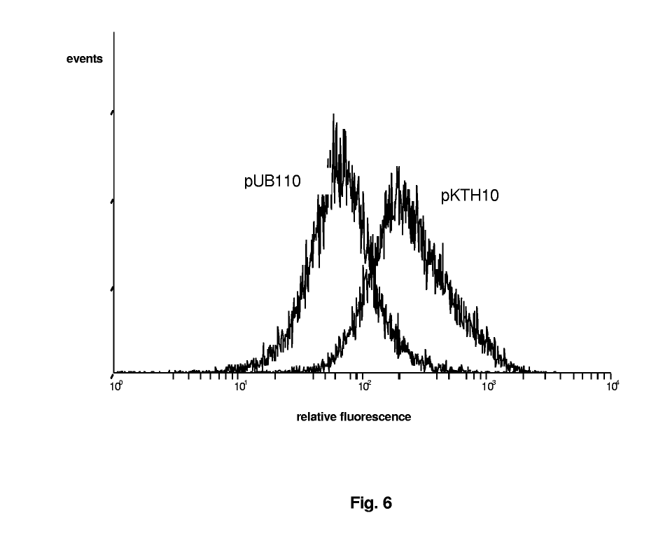

The plasmids pUB110 (empty control vector) and pKTH10 (encoding Bacillus amyloliquefaciens a-amylase gene amyQ) were transformed separately to Bacillus subtilis strain VT210B. One part of the cells was spread on selective TY agar plates, and to the other part 2×TY broth containing kanamycin, was added and the cells were cultivated over night. Cultures were analysed by a skilled operator on a flow cytometer (Coulter Epics XL-MCL, Beckman Coulter). A typical experiment comprised analysis of 20.000 events (cells). Fluorescent signal was detected using a FITC-filter and setting of the photo multiplier voltage was between 700 and 800 Volts. The difference in fluorescent signal between the normal cells and cells stressed by secretion is depicted in FIG. 6.

### 2.3 Selection for Secretion Stressed VT210A Cells Using a Fluorimeter:

Transformed reporter strain cells were inoculated in 2×TY broth in the presence of kanamycin and grown over night at 37° C. The DNA's used for transformation were equal amounts of the plasmids pUB110, pKTH10, pUBnpr2 and pUBBAG. After growth to stationary phase the cultured cells of the four transformations were divided 8-fold in equal volumes in a 96 well micro titer plate format. The wells were analyzed for fluorescence in a fluorimeter (Molecular Devices, type Spectra MAX Gemini). Excitation is at 490 nm, emission at 510 nm, and cutoff at 495 nm. A standard t-Test assuming unequal variances was performed. Table 2 demonstrates that non-stressed cells (pUB110) were distinguished from the stressed cells (PKTH10, pUBnpr2 and PUBBAG) by a clear significant (P two-tail<0.05) difference in fluorescent signal.

TABLE 2
t-Test assuming unequal variances.

| | pUB110 cells | Ratio |
|---|---|---|
| pKTH10 cells | P = 1.05899E-13 | 7.84 |
| pUBnpr2 cells | P = 4.63415E-11 | 1.58 |
| pUBBAG cells | P = 7.17216E-14 | 1.77 |

P-values are given for empty host cells (pUB110) versus secretion stressed (pKTH10, pUBnpr2, pUBBAG) cells.
The ratio expresses the mean (n = 8) fluorescent value of stressed cells over the mean (n = 8) fluorescent value of empty host cells.

### Example 3

### Genome-Wide Expression Analyses of A. niger to Identify Secretion-Induced Promoters

Genome-wide expression analyses were performed to identify promoter sequences that are highly expressed in A. niger WT-PHY, and show no or low expression in A. niger WT-GFP, A. niger WT-vector and A. niger WT-1. To simulate future application experiments protoplasts were obtained of A. niger WT-1, A. niger WT-GFP, A. niger WT-PHY and A. niger WT-vector strains. An amount of 20 ml liquid selective regeneration medium supplemented with 50 mg/l phleomycin (Invitrogen) in case of A. niger WT-GFP, A. niger WT-PHY and A. niger WT-vector, was inoculated in a petri dish with 100 microliter 10E7/ml protoplasts. After a growth period of 3 days at 30 degrees Celsius (static), a mycelial pellet had formed at the surface of the liquid medium which was used for total RNA isolation. cDNA synthesis, labelling of the cDNA and hybridisation on Affymetrix A. niger GeneChips™ was performed according to suppliers protocol. Subsequent expression analysis resulted in identification of 5 genes that had an expression level in the A. niger WT-PHY strain of at least 8.5 times the basal expression level in A. niger WT-1, A. niger WT-GFP and A. niger WT-vector. To confirm the findings, Northern blot analysis was performed.

### Example 4

### Northern Analysis

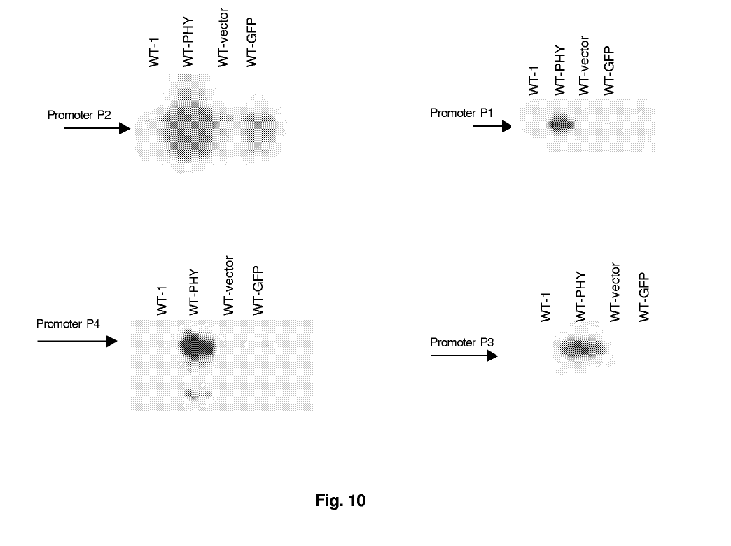

To corroborate the results of the Affymetrix A. niger GeneChips™ experiments, Northern blot analysis was performed. For this, total RNA was isolated from A. niger strains WT-1, WT-GFP, WT-FYT and WT-vector. RNA was denatured, separated by gel electrophoresis on a 1% agarose gel and transferred onto a nylon membrane (Hybond-N₊, Amersham Biosciences) by capillary blotting according to the manufacturer's instruction. The RNA was UV cross-linked to the nylon membranes on a GS Gene Linker™ (BioRad, C3 setting, 150 mJoule). DNA for the generation of ³²P-labeled DNA probes was isolated by PCR using genomic DNA of A. niger WTF-1 as a template and using the primers listed in Table 3 (SEQ ID NO's 50 to 59). Northern blots were hybridised with randomly primed ³²P-labeled DNA probes, representing the secretion-induced genes (SEQ ID NO's 20 to 24), according to suppliers instructions (RadPrime DNA Labeling System, Invitrogen). The results of the Northern analyses are shown in FIG. 10. The hybridisation pattern of the Northern blots confirm the data obtained by expression analyses of the Affymetrix A. niger GeneChips™ data; low expression of the secretion-induced genes in A. niger strains WT-1, WT-GFP, WT-vector and high expression in A. niger WT-PHY.

TABLE 3

Primers used to isolate
DNA for 32P-labeled probes.

| | | |
|---|---|---|
| Primer | Primer sequence | Gene sequence |

| | | |
|---|---|---|
| SEQ ID NO: 50 | CAACACCTACGGCGTCAAGG | SEQ ID NO:20 |
| SEQ ID NO: 51 | TAACCCGGGGAGATGCTGTT | SEQ ID NO:20 |
| SEQ ID NO: 52 | CGATCCGGAAATTCCTCCTG | SEQ ID NO:21 |
| SEQ ID NO: 53 | GCCGCCGACTCAGCAGTAT | SEQ ID NO:21 |
| SEQ ID NO: 54 | GTGCATGAACAACACGGAGA | SEQ ID NO:22 |
| SEQ ID NO: 55 | ACTCAGTVGTGCGCCTCTCG | SEQ ID NO:22 |
| SEQ ID NO: 56 | GTCGAACTCGGCGTVTCTCC | SEQ ID NO:23 |
| SEQ ID NO: 57 | CCGGTGAGGAGGTGCTTVTC | SEQ ID NO:23 |
| SEQ ID NO: 58 | AGCATCTCGCCATCCATCAG | SEQ ID NO:24 |
| SEQ ID NO: 59 | GCCTTCGGACCATGGTATCC | SEQ ID NO:24 |

### Example 5

### Cloning of GFP-BLE Secretion-Induced Reporter Constructs

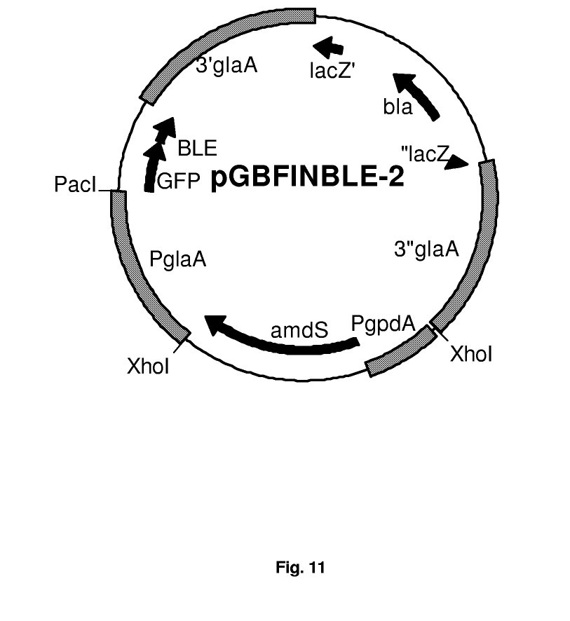

A fusion of GFP (Green Fluorescent Protein (Chalfie, M et al., Science (1994) 263(5148): 802-805) and phleomycin binding protein (BLE, present in pGBFIN-14 previously described in WO99/32617) was constructed by means of PCR. The GFP fragment was amplified by PCR using the primer set consisting of SEQ ID NO: 62 and SEQ ID NO: 63. The BLE fragment was amplified by PCR using the primer set consisting of SEQ ID NO: 64 and SEQ ID NO: 65 with pGBFIN-14 as template. The overlapping PCR fragments obtained were used in a fusion PCR with primers SEQ ID NO: 62 and SEQ ID NO: 65, resulting in PCR fusion product GFP-BLE (SEQ ID NO: 61). The GFP-BLE fusion was digested with restriction enzymes PacI and AscI and ligated in the PacI, AscI linearized cloning vector pGBFIN-2 (described in WO99/32617 and EP98/08577) thereby placing the GFP-BLE fusion under control of the glucoamylase promoter, the resulting vector pGBFINBLE-2 is depicted in FIG. 11.

Promoter sequences of the secretion-induced genes were isolated by PCR using genomic DNA from A. niger WT-1 as a template and primers as displayed in Table 4.

TABLE 4
Overview of primers used for amplification of corresponding promoters

| | | | |
|---|---|---|---|
| Primer | Primer sequence | Promoter | PCR product |

| | | | |
|---|---|---|---|
| SEQ ID NO:40 | ACTTCATTAATTAAGTTGATTGAGGTAGAGATGAGTTTG | P1, SEQ ID NO:20 | SEQ ID NO:35 |
| SEQ ID NO:41 | ACTTCACTCGAGATAATGTAGGCGACAAAGTAGCC | P1, SEQ ID NO:20 | SEQ ID NO:35 |
| SEQ ID NO:42 | ACTTCATTAATTAATTTGACACTTAACGTGGTAAGG | P2, SEQ ID NO:21 | SEQ ID NO:36 |
| SEQ ID NO:43 | ACTTCACTCGAGTTCTCACAAGCAAATCCGAGA | P2, SEQ ID NO:21 | SEQ ID NO:36 |
| SEQ ID NO:44 | ATCCTACTCGAGTGGTGCCTGTGAACGAGGTCA | P3, SEQ ID NO:22 | SEQ ID NO:37 |
| SEQ ID NO:45 | ACTTCATTAATTAATGTGGATTTTGGATAGTAATTAAAG | P3, SEQ ID NO:22 | SEQ ID NO:37 |
| SEQ ID NO:46 | ATCCTATTAATTAAGATGGTTTGGTGTATAACAGAACA | P4, SEQ ID NO:23 | SEQ ID NO:38 |
| SEQ ID NO:47 | ACTTCACTCGAGGTCCACACTCTAATTGGGATGA | P4, SEQ ID NO:23 | SEQ ID NO:38 |
| SEQ ID NO:48 | ATCCTATTAATTAAGTTGCATCTGCGACAGACAGT | P5, SEQ ID NO:24 | SEQ ID NO:39 |
| SEQ ID NO:49 | ACTTCACTCGAGGCCTTTGGAGMTGTAATATCCC | P5, SEQ ID NO:24 | SEQ ID NO:39 |

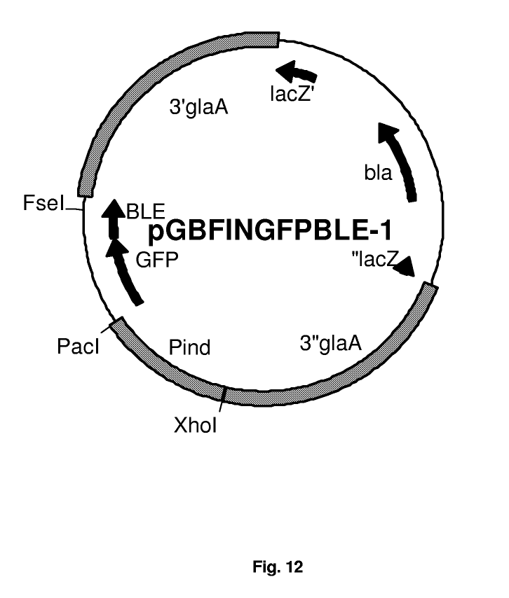

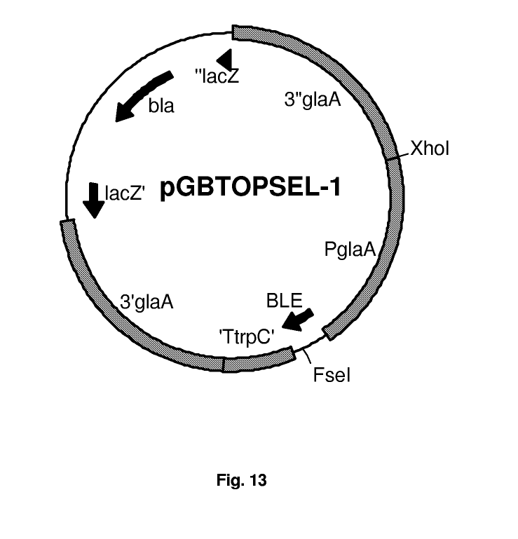

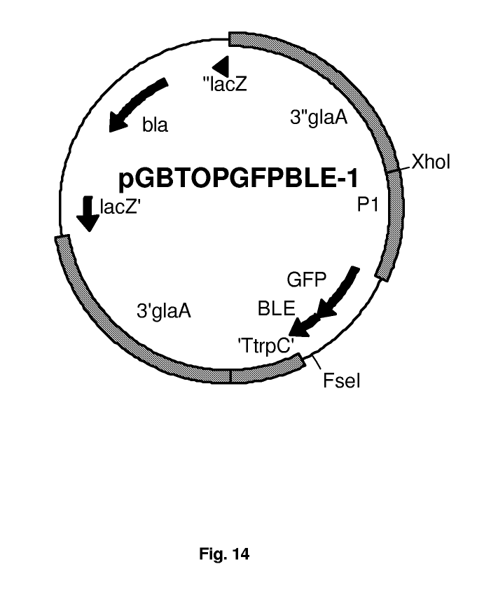

The resulting PCR fragments (SEQ ID NO's: 35 to 39) were digested with restriction enzymes XhoI and PacI and ligated in the XhoI, PacI linearized cloning vector pGBFINBLE-2 depicted in FIG. 11, thereby removing PgpdA-amdS and replacing the glucoamylase promoter with the respective secretion-inducible promoter, Pind, resulting in pGBFINGFPBLE-1 to 5. pGBFINGFPBLE-1 is depicted in FIG. 12, and is representative for pGBFINGFPBLE-2 to 5. The fusion constructs of the respective inducible promoters with GFP-BLE was isolated by XhoI and FseI digestion of pGBFINGFPBLE-1 and subsequently ligated in the XhoI, FseI linearized A. niger expression vector pGBTOPSEL-1 depicted in FIG. 13. The resulting secretion reporter constructs, pGBTOPGFPBLE-1 to 5, contain a fusion of GFP-BLE under the control of respectively secretion-inducible promoter P1, P2, P3, P4, P5. In FIG. 14 pGBTOPGFPBLE-1 is depicted as representative for pGBTOPGFPBLE-1 to 5. These vectors were used to transform A. niger WT-1 in example 6 and 7.

### Example 6

### Overexpression of Extracellular Protein Confers Phleomycin Resistance to A. Niger Expressing a GFP-BLE Reporter Construct

Transformation of the secretion reporter constructs pGBTOPGFPBLE-1 to 5 to A. niger WT-PHY-2 overexpressing the secreted protein phytase was performed according to the procedure described in Materials and Methods. Transformants were selected on selective regeneration plates supplemented with various amounts of phleomycin (0, 5, 10, 15, 20, 25, 30, 35, 40, 45, 50 microgram per ml) as selective agent. All 5 reporter constructs enabled direct selection of transformants on the phleomycin containing plates whereas the negative control, WT-PHY-2 wild-type, was not able to grow on any of the plates containing phleomycin. The secretion reporter constructs pGBTOPGFPBLE-1 to 5 enabled growth on respectively 50, 35, 50, 35, 25 microgram per ml phleomcyin. Thereby demonstrating that the secretion-inducible promoters of these constructs are functional and direct selection on phleomycin after transformation is possible.

### Example 7

### Selection of A. niger Clones Expressing Extracellular Proteins after Co-Transformation of a cDNA Library and the GFP-BLE Reporter Construct

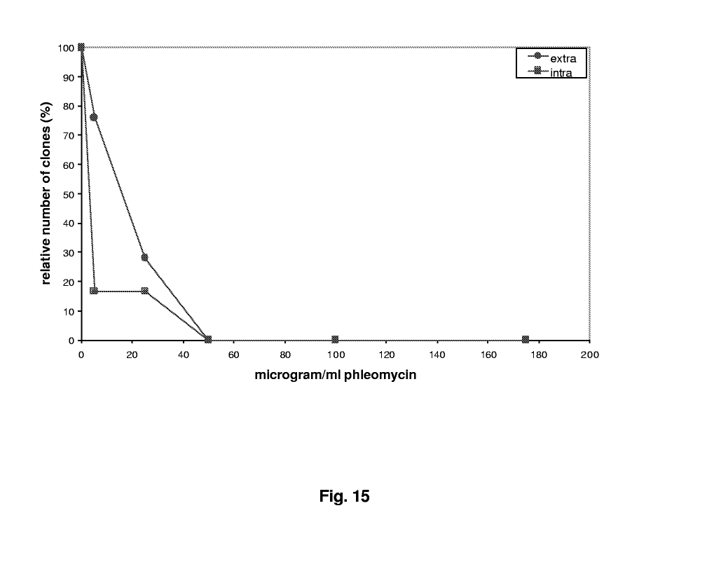

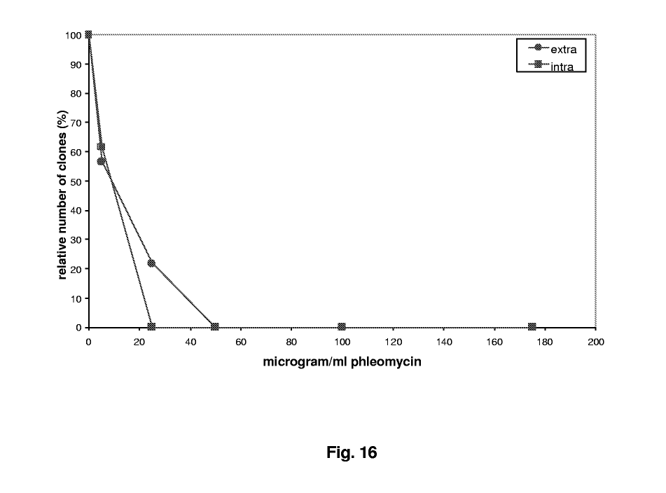

Aspergillus niger WT-1 was co-transformed using the secretion reporter constructs pGBTOPGFPBLE-1 to 5 in combination with a defined A. niger cDNA library encoding for 50 extracellular proteins and 50 intracellular proteins. Co-transformation events were confirmed by colony PCR. Spores of co-transformants containing the secretion reporter construct in combination with an expression library construct were grown on selective plates supplemented with different amounts of phleomycin (0, 5, 25, 50, 100, 175 microgram per ml). An average increase in phleomycin resistance was demonstrated for co-transformants containing the secretion reporter construct in combination with a library construct encoding an extracellular protein compared to transformants with the secretion reporter construct in combination with a library construct encoding an intracellular protein. Graphics representing these results for secretion sensitive promoters P4 and P5 are shown in FIGS. 15 and 16. These results demonstrate that an increase in phleomycin resistance of at least up to 20-fold was achieved.

Diversity of extracellular proteins produced by the co-transformants which contained secretion reporter constructs comprising promoters P1 or P3 in combination with a library construct encoding an extracellular protein was demonstrated. Co-transformants were individually grown in microtiterplates and on day 6 the supernatant of these cultures was subjected to protein analysis by E-PAGE™ High-Throughput Pre-Cast Gel System (Invitrogen) and mass spectrometry (MALDI MS/MS).

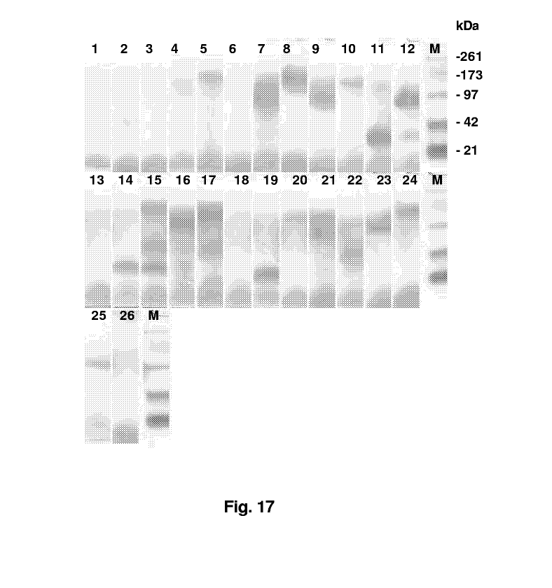

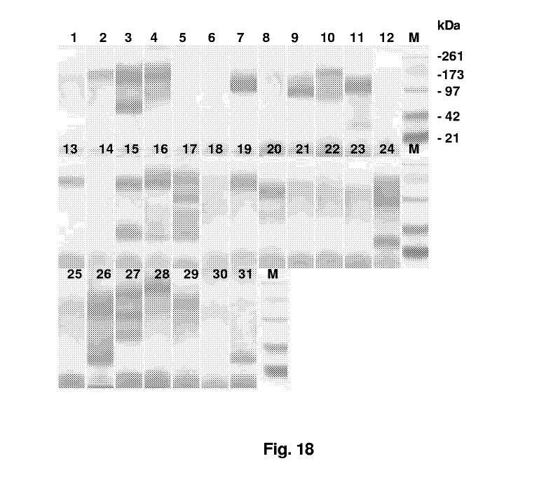

The E-PAGE™ 96, 6% Gels (Invitrogen) contain 96 sample lanes in microtiterplate format and 8 marker lanes for protein separation. Supernatant and marker (E-PAGE™ SeeBlue® pre-stained standard (Invitrogen)) were loaded on the gel according to suppliers manual. After electrophoresis of the gel, SimplyBlue™ Safestain (Invitrogen) was used for staining according to suppliers manual. The scan of the E-PAGE™ gel was edited by the E-editor program provided by Invitrogen, which aligns the protein samples in microtiterplate format. Extracellular proteins produced by co-transformants with secretion sensitive promoters P1 and P3 are displayed in FIGS. 17 and 18, and are summarized in Table 5. These results demonstrate that up to 77% of the analysed phleomycin resistant colonies secreted proteins in amounts detectable by E-PAGE. Based on the size of the observed protein fragments on the gels, the diversity of the clones transformed with the defined library was estimated. These results demonstrate that a diversity of up to 35% could be observed based on E-PAGE analysis.

The mass spectrometry results are depicted in Table 5. Up to 58% of the analysed phleomycin resistant colonies secreted proteins in amounts detectable by mass spectrometry. Diversity was estimated based on the identity of the detected peptide sequences. These results demonstrate that a diversity of up to 100% could be observed using mass spectrometry.

TABLE 5
Summary of E-PAGE and mass spectrometry results.

| | | Transformants | | | | |
|---|---|---|---|---|---|---|
| | | analysed | Positive | | Diversity | |
| Assay | Promoter | No | No | % | No | % |
| MALDI | 1 | 26 | 14 | 54% | 14 | 100% |
| MALDI | 3 | 31 | 18 | 58% | 12 | 67% |
| E-PAGE | 1 | 26 | 20 | 77% | 7 | 35% |
| E-PAGE | 3 | 31 | 20 | 65% | 6 | 30% |

The assay used for analysis is depicted in the first column;
the second column identifies the secretion sensitive promoter-reporter construct;
the third column depicts the amount of transformants that were analysed by either assay;
the fourth and fifth column depict the amount of colonies scored positive for secretion of a protein and the respective positive percentage;
the sixth and seventh column depict the number of colonies secreting distinct proteins and the respective diversity percentage.

1. A promoter DNA sequence selected from the group consisting of:
(a) SEQ ID NO:1, SEQ ID NO:2, SEQ ID NO:20, SEQ ID NO:21, SEQ ID NO:22, SEQ ID NO:23, or SEQ ID NO:24;
(b) a DNA sequence capable of hybridizing with a DNA sequence of (a);
(c) a DNA sequence being at least 50% homologous to a DNA sequence of (a);
(d) a variant of any of the DNA sequences of (a) to (c); and
(e) a subsequence of any of the DNA sequences of (a) to (d).

2. A DNA construct comprising a promoter DNA sequence according to claim 1 operatively associated with a reporter gene conferring a selectable trait, preferably a selection marker gene.

3. An expression vector comprising a DNA construct according to claim 2, wherein the DNA construct comprises a DNA fragment, which is homologous to a DNA sequence in a predetermined target locus in the genome of a host cell, preferably the predetermined target locus comprises a highly expressed gene.

4. An expression vector comprising a DNA construct according to claim 2, wherein the DNA construct is capable of autonomous maintenance in a host cell, preferably the DNA construct comprises an AMA1-sequence.

5. A host cell comprising the DNA construct according to claim 2, or comprising a expression vector comprising the DNA construct.

6. The host cell according to claim 5, further comprising a DNA construct comprising a DNA sequence comprising a coding sequence originating from a DNA library from an organism suspected of being capable of producing one or more proteins of interest.

7. The host cell according to claim 5, wherein the host cell is a prokaryote or an eukaryote.

8. The host cell according to claim 7, wherein the host cell is selected from the following list: a Bacillus, a yeast or a filamentous fungus, preferably an Aspergillus, Penicillium or Trichoderma species.

9. The host cell according to claim 8, wherein the Aspergillus is an Aspergillus niger or Aspergillus sojae or Aspergillus oryzae species.

10. A method for isolating a DNA sequence comprising a nucleotide sequence coding for a protein of interest in a host cell, said method comprising:
(a) preparing a first DNA construct comprising a promoter DNA sequence operatively associated with a reporter gene conferring a selectable trait; said promoter DNA sequence being induced when the DNA construct is present in the host cell and when a protein of interest is produced by the host cell;
(b) preparing a second DNA construct comprising a DNA sequence comprising a nucleotide sequence coding for a protein of interest originating from a DNA library from an organism suspected of being capable of producing one or more proteins of interest;
(c) transforming a host cell with both DNA constructs prepared in (a) and in (b);
(d) culturing all the transformed host cells obtained in (c) under conditions conducive to the production of the proteins of interest as present in the DNA library; and
(e) screening for transformed host cells producing a protein of interest by analysis of the proteins produced in (d).

11. The method according to claim 10, wherein the host cell is first transformed with the DNA construct prepared in step (a) and consecutively is transformed with the DNA construct prepared in step (b).

12. The method according to claim 10 wherein the promoter DNA sequence used in step (a) is selected from the group consisting of:
(i) SEQ ID NO:1, SEQ ID NO:2, SEQ ID NO:20, SEQ ID NO:21, SEQ ID NO:22, SEQ ID NO:23, or SEQ ID NO:24;
(ii) a DNA sequence capable of hybridizing with a DNA sequence of (i);
(iii) a DNA sequence being at least 50% homologous to a DNA sequence of (i);
(iv) a variant of any of the DNA sequences of (i) to (iii); and
(v) a subsequence of any of the DNA sequences of (i) to (iv).

13. The method according to claim 10, wherein the organism suspected of being capable of producing a protein of interest is a eukaryote or a prokaryote.

14. The method according to claim 10, wherein the protein of interest is an enzyme.

15. The method according to claim 10, wherein the protein of interest is a secreted protein.


SEQUENCE LISTING (65 sequences)

SEQ ID NO: 1 - DNA, 168 bp, ORGNISM: Bacillus subtilis
cgactcagtcctttcatatacaatatgaagtgtaccgttttccgcactttttcacaatttcccataatcttttcatttttatcccacagtttttgtttatgataaactcaagtcataaacctatcaatataaatagacatgtgaaaatagagaaacggagtgaacatg

SEQ ID NO: 2 - DNA, 277 bp, Bacillus subtilis
ggctcttcacatcctttcaacgtcattataaactagttttaacatacggcaggcaattttcataatttcacatattcttttcatttttatcccacaatgtttgtttatgatatggttaagggaagaaaggaagagaaaaagagcgaggaagatgtaggatgatcaaacatattctattttggtgctttgcttttctcctcattattgggacaattgaacttgtacatgcgatgaatatgtaacgataaatgaataaccgttaaaggagtgtaagaac

SEQ ID NO: 3 - DNA, 26 bp, Artificial Sequence
  primer
gtgagcggataacaatttcacacagg

SEQ ID NO: 4 - DNA, 70 bp, Artificial Sequence
  primer
gtggctcagcttttttaaggaagggaggctctcacctcccttccctttatttgtagagctcatccatgcc

SEQ ID NO: 5 - DNA, 27 bp, Artificial Sequence
  primer
cgtgaggtaccggcttctgtttctgcc

SEQ ID NO: 6 - DNA, 32 bp, Artificial Sequence
  primer
catcacgaagcttatccatcatgttcactccg

SEQ ID NO: 7 - DNA, 25 bp, Artificial Sequence
  primer
gaccggtacctcaggatctttcgcc

SEQ ID NO: 8 - DNA, 35 bp, Artificial Sequence
  primer
ggccatcaagcttataatccatgttcttacactcc

SEQ ID NO: 9 - DNA, 19 bp, Artificial Sequence
  primer
gaaagattgtttcagaagc

SEQ ID NO: 10 - DNA, 40 bp, Artificial Sequence
  primer
acagcgttgggatccaagcccttccattttggacatttgg

SEQ ID NO: 11 - DNA, 40 bp, Artificial Sequence
  primer
gggcttggatcccaacgctgtcgacgttgtaaaacgacgg

SEQ ID NO: 12 - DNA, 42 bp, Artificial Sequence
  primer
cgcatagctttccggtcgccgcagctatgaccatgattacgc

SEQ ID NO: 13 - DNA, 22 bp, Artificial Sequence
  primer
gatgattcgtctttttgtagtg

SEQ ID NO: 14 - DNA, 21 bp, Artificial Sequence
  primer
cgatcaacatatgtgttcacg

SEQ ID NO: 15 - DNA, 1050 bp, Aspergillus niger
ataatgtaggcgacaaagtagccgggcatgcgaccggaggactcggtcagaggcacgaatatgagaggccagaacgccgcacccatgttccaagatgttgtggcccagtaaaggttaggaaatggggtgttttgaacgtggaatcgttcttccatccagttgttgccggagccgtaggaaccggccgggataccagtgaggatggaactatacctattagcactgccatcttcgagaacaaggctaagacatacatgaaacaaacggtgaccgtgagaagccatttataagaggtgctccaattgaacctgaatatgttaaaactagtatactagcaactgcatgcttgacgtacggattgtccgggtcatccttgccgttccaggtatggatctcaaggtcctccctgtcacgcagatcattgtggctgactaccccgacatgggggtcccggcgaaagaacgaccgcaccctgcaacgtcacttagtggctgtctgacagggattgattggcaatgattggcagactaaccgtcccagaaacgaatcatcgaactcattggtgtgcactgtgctagcgcggcgcagcgacgggcgtggctcctcggcaggggtggacatgactttctctgtgtagattccttgcaaggtatctgcttgacagatgcagagatgatgttgagtaaaatagggtgcagagaccctgcagaacctgcatcggccggcggcgacatgtcgtcatgaccgacccgggccgagcggaattaatgaaactcggaacagccaggggaatccgacgctttgagtggccaatccatgtctatccacatctgcattgaagtggatgaaaggtggaggagggctggttgccaaattctgttagggcttggaatgtaagcaaccggtttcggtcatgacatcatcacagcgatgacttcatcactctcggacgagcatatatagttgtgcctgtcgtcgtatctcaacaaacatcaacaacaacaacaatcatctactgcatttaccaaactcatctctacctcaatcaac

SEQ ID NO: 16 - DNA, 1032 bp, Aspergillus niger
ttctcacaagcaaatccgagaaacgcaattgaccatatgtccttagtatgagggctagcaggaatacttgtttgtcgatgcataccgactaataaaaatggttcttctttttcgcgcagggatttggtttttcatttttcatttttcatattttatgttattttattttatttttttgttttattttattttattttttttttttaaataagactgggtgttttcgtctcccacccctgctgaggaactgtttgatatacagaagcctgagatacttgagattatgtgacaaagaaattgtaaacctgaatgtgactaaggcagaagagagcaggtgaggccacacaatactgtacacccacttccgatcgtcatcgaaatgagcgttgtgggtactggaacgacaaaccatagatcggatctgcatccgaataggctttactttgtacattggtaggagtttgaatgtttgaagatccggcagatgatcacctgctgcagggaataatttgaaacctgtccgtctgctttgacccaattgacgaggtacgtgatttgatgctacccaagcagtatgaatgcgacggaggtggctatccttaccttgtcatatgcaactcataacgggtagtccccatggagccacggctatttatttctaagggccaacagtgaaagattgtgagatcgtatacctcctgggtatgacgcaaccagggtgagcactggcattgctccaatccatatggtggaggtatgtctccgagaccatgacgcatgtagaccctgacctgtccctgatccagggatctggtttgtcacttggcgtattttaattaactgcgggtcagggtcttgccgtccagacccaatggatgaattgcctgcgtggccctgcagttccagcgatcggcacggcacggctatgacgtcggtgggtgttccgtttgatatttaaccatagaagacagccttgttatgctggttcctgccgcaaaataaaggtctatccttaccacgttaagtgtcaaa

SEQ ID NO: 17 - DNA, 1035 bp, Aspergillus niger
tggtgcctgtgaacgaggtcaccactgataaattgtctggatccgtaacgtctgtatcagtggatggtactgaaaagttccaaagattgagggcagcatatgtagcacatggtggtagctctaagtggttcgaaacttcgatgaatgggcgtgatatacaggggggtagcacctgtatagttgatgtcagcgaaggcacgggccatgcagtgatagacaagaaaagagggattgaataaaagactcgattctgaaggggagtaatacatctttcggcttctcaccaccccagatgtaggcgtgtgtaaggtaagcaagcacaacatatgctctccgccactcaggttcccccttcaacttcttcgtagagagtatacccaggtcatcgacagacctcctgatagtaccagactgaatcaggccaggcaagtcctgagcgatttcctcccacggggaatagtaagggtcttccaatctccgtacgggaggaacttccgggaggaacccgttctggagggagacaccgtacgcaacaagtgaaatgtcttgtgtataaagcatgataatatatgcacttcagtagacaacttgatattcaagcctccttgaaatgatcctcatgcaggtaagagtagatccaagtgtttatattgtattcccaaggagggacggaagaaaaccggatccagagggctcgagatagggccacctgcatgagcaaagacgggtcatttcccgtttcacaacgaccattctcaggcagaaaggcagtggattagcggaaaacttagcaaccatccggatttctggagtggatgctttgttatctcacttccggcaacacggatggtgaggcggaagcggaacggtatccggatccggagggagggacaaaccgaagtgccttgtgtcacacgccggtccatatataaatgttaattggtgacggtcagcggattgtttctggtgttcatccattttttcttgatatcctggacccagttattgcactttaattactatccaaaatccaca

SEQ ID NO: 18 - DNA, 1071 bp, Aspergillus niger
gtccacactctaattgggatgagcattgcctttcaggaacttgactttttgtggttgcatcttttgttgccttacccacgcaggcttcgtttatgagatagcgatttcaggcctcaaattgctgtcatcacttccggccccaaactcgtgggcggtgcccgcccatcgctggcaagaagcacagactaacgccttacttcgcctagttgcacacgatgcatcaagacttcctcctatttcacaacgaccagctccttgccatttgttgtttcgaatgcccccttccccgtgccttgcatttctaattcccatctatacccttttgcgctctcacgcccgcctgactatcaatttttctgccagcataaccctgcgtgcagagcggttgacagcaggcttgctaagagtcttcgtctagaagaactgtctgggtggattccttttcggattaacttttgctattttaaaagctgattgcactacggagtacacccgcaatctgattctcactgatactccaattaaaggccactaattgctgacatttgtaacatatttgcgtttgctttccggttgaaattggcggctttgtgctttcgatcgctagcagtctctgtgcatgtacaactgatcggctattcagtcctattcggaagcggcactgagaggcggagacaagccacagatacgacgagtctgattttgagacgggcgcatccaacgaaagcctgcgattgcaattgaagatctcaattgggggactgacaacctaatacctaaacatatcgaggattaggcgccaacacgagcatgggaagctttggtgcatgcgacggctgatccatttctaacaaccgttttgcgcgcgcgtccgaggtaaggctcccgatcccttggcgctttgcatgtttcactccgatgcatctactgtttatatgagtgcggtgggcaagctgtgcccaccgactgagcattctcaaccggcccaatgctgtgggatcgatcctcccctccaaagttgtcgctcggttacacttgatcggagcttgttctgttatacaccaaaccatc

SEQ ID NO: 19 - DNA, 1116 bp, Aspergillus niger
gcctttggagaatgtaatatcccttgatcaccccagactcacatccagattctgactgacctcgtgcatagccatatttaacttggtccctgatgatcaaaacagtacatggaactggggccgaacatttccctagccaaaggagcatcagggattcaacgtgatcagtacagccttgtagctcgctgaaactgccctggagccaagccaaagcctagacagcaaggtgcatgggcgagctttgcgaggatttagttgttatttagtatgccaagattaagccacgcagaatggcttatggccatagtagcaacaacggccacggaacgttcttccatgtggtatggggaaagccgccggatatttggggagggcgtgattgttccggtggctatctttccggtggagataacggcggtggaccatcaaaactcctcagcccggattgctcggactccgcatcgtcaagtatcggtttcatgacagccattcgactacttagaagacacaaatagctatccaacttacccggacctttgataataattttcagcagatctgtcgatctcagagacaataatgccaccgttctgtgtggaaggcactgcgccgtggtttaacaataataaaactccaaattgcccaaaagctagagcgaaggaaagtcgagaagcccagtctagactggcgggctgatcgtccggttcacaacctgattgacaaatcatctgtgaccctcgatgttccgcggcgttgagcgggtgagaatgtggatcctagtggggaagacccaattgccgccggaaaaggtgagcttgtgctgcacatgtccggtggctcaattcagggccatgccaggacattgctcttcaaagggatgcgattgctattccctatttacttctttaacccgctgacgggcctgtctggaccgagaattcttggtcaagtttcagtggagcgatccaggaaagaacatccgatatggacagttgccctcgatctcaggtctgtagtcgtgtgacacagaggcctctactcgcttgccttgcatgtcggagctcaaaggtgcgatgtgaattaacacgaggagcgactgtctgtcgcagatgcaac

SEQ ID NO: 20 - DNA, 3191 bp, Aspergillus niger
ggagttacgtcccagagatgctgcggacaatgccgatgcggcatacttctcatacgcatccgtcaagtaattcacaacagccatgtagatactgtagatgccaataccggtagccccgatgccgagggtcgggacaatccaaggcacagaaggaaaactggcccaaccataccagaacagcccgcctgcgaacagcaagctgccaatgatactagtatacagacgggcctccgggatgggtttcccgggcttctctttgttgcgtttcgccgaccgcaaatacagcatatcctgaagaggattgacgagagtaccgattagagcacccacggagatggccaattgaatgcacccagtttgcaaagtattcatgccgtagttggtggagaaggtctgaacgacactgctgaagaagagatacaggataccccaggcgaaagagatccagagggtgaagaaggcaacgacgggctcggtgaggagcattcgtgtgggccgttcgaaggagatctgcatcagacgccagacgctggtgttgtctagctcggcttcggcgtagatggggcgctgcttttctttgcggagcttcttggcccgacgggtgaggatcacgtccgcgcgggtttcgtacaggatgaaccagaagatgggtaggagccctgttagatagataatttggatgtagaagatccagcgccacggggcggttttgtggatggctacgatggcggatccgatgaatgggcccagggcaattccaaccacactggtaaaaccgaagagcgacatagggagactcctcgctttgtcaccataccagacatcactgatgctaccgccgacgatgttgatcgatacggaggacgcaccaccaccgaagaaacgcgtcactacgagagtagcgtagttttgggcgaaggcggaggggaatagcgagatgatgagaataatgtaggcgacaaagtagccgggcatgcgaccggaggactcggtcagaggcacgaatatgagaggccagaacgccgcacccatgttccaagatgttgtggcccagtaaaggttaggaaatggggtgttttgaacgtggaatcgttcttccatccagttgttgccggagccgtaggaaccggccgggataccagtgaggatggaactatacctattagcactgccatcttcgagaacaaggctaagacatacatgaaacaaacggtgaccgtgagaagccatttataagaggtgctccaattgaacctgaatatgttaaaactagtatactagcaactgcatgcttgacgtacggattgtccgggtcatccttgccgttccaggtatggatctcaaggtcctccctgtcacgcagatcattgtggctgactaccccgacatgggggtcccggcgaaagaacgaccgcaccctgcaacgtcacttagtggctgtctgacagggattgattggcaatgattggcagactaaccgtcccagaaacgaatcatcgaactcattggtgtgcactgtgctagcgcggcgcagcgacgggcgtggctcctcggcaggggtggacatgactttctctgtgtagattccttgcaaggtatctgcttgacagatgcagagatgatgttgagtaaaatagggtgcagagaccctgcagaacctgcatcggccggcggcgacatgtcgtcatgaccgacccgggccgagcggaattaatgaaactcggaacagccaggggaatccgacgctttgagtggccaatccatgtctatccacatctgcattgaagtggatgaaaggtggaggagggctggttgccaaattctgttagggcttggaatgtaagcaaccggtttcggtcatgacatcatcacagcgatgacttcatcactctcggacgagcatatatagttgtgcctgtcgtcgtatctcaacaaacatcaacaacaacaacaatcatctactgcatttaccaaactcatctctacctcaatcaacatgcctatctccattccctccgcctccagcgtccacgacctcttcagcctgaagggcaaggtcgtcgttatcaccggcgcttccggccctcgcggcatgggcattgaagccgcgcgtggctgcgccgaaatgggcgccaacatcgccctcacctactcctctcgtcctcagggtggtgagaagaacgccgaagaacttcgcaacacctacggcgtcaaggccaaggcctaccagtgcaacgtgggcgactggaacagcgtcaagaagctcgtggacgacgtgctggccgagtttggccagattgacgccttcattgccaacgctggcaagacagccagcagtggtatcctggatggttccgttgaggactgggaagaggtcatccagacagacctgacgggtaccttccactgcgccaaggcggtgggcccgcacttcaagcagcgcggaacgggcagcttcatcatcacctccagcatgtcgggtcacattgccaacttcccgcaggagcagacgtcgtacaacgttgccaaggcaggctgcatccatatggcccggtcattggccaacgagtggagggactttgcccgcgtgaacagcatctccccgggttacatcgacacggggctgtcggactttgtggataagaagactcaggatctgtggatgtcgatgatccccatgggacgcaacggtgatgccaaggagctgaagggtgcatatgtctatctcgccagtgatgccagcacatacacgacgggtgccgatttggtcattgacggaggatacaccgtgcggtaaattatcatccgcggtcggatagaaaataatgtttatgacatgtatgtaatgaatatgaatgttcaacaatagtcttcaattcttcacttgctagtatcctgagtatcttgtcgtatagctcaacaacctggcatggccgggcgcgcagtgtagctctcccaagtctatggatacaacttgcgtcagtgtggatctttggctcagattcggaggggcagtggggtaaacaagaacacgcagccactctcggctttgcggatccccacggaacgccagtcaaccctgaagattcagtccgggcctgggctcggactgatttggtgaggggattctgcatccgtccaaaccttatcatcgttggtgagacggactccggacagccaggagccttatt

SEQ ID NO: 21 - DNA, 4031 bp, Aspergillus niger
ggggtcaaaggacacaatgtcgaaatagcgagattgctgtgcatcatcggggctgtctacagtccgctggatttctttccctcggttaagtatttcctgcacgccggacccgccagggccacctgtattcctagtaagcgaccgtggtggggtgacagaatggtggcggtggtggcattgctgcctatacctgggttcacgattatggagccaccgtaacgctcgtcggcaggatctacctgtgcaggcagttttatgacggccagggcagccgttttattttcaatggcactagctgaccagtctaggggaacttcgagacgtgcacatagatagtcctggtagcaggggatgaaatttagtgctgagcctggcgcagtctggtaggagcgggttagactgtgagaaattataatagggcggatgagatcagtacgctactccaggagaagtccccggcttgcaccgcagacggcatcatgggttggtgtcgcagggaggagggtgagttgagtaagagagaattaatgaaaaggatggagtagccgaccatataagtactggatgaagtgagcgagttagcgtcagccacgacgggctgacatgcatgatcggggcaccgccggcaaccgatacagcctcagctcgacgggaaaacgtggttacctggacttagaagatgataatacaatgaaaagatagcgtagattgccataaatacgccacaatctatttattaggcaacaatcaagtaagagcagatttggcgactcatagtagtccccatggaggggcgcgatggttttcttcgccgaaataatcgcttaatgcctctaacgaagatctcagaacctccaaactgtagtattcttctcacaagcaaatccgagaaacgcaattgaccatatgtccttagtatgagggctagcaggaatacttgtttgtcgatgcataccgactaataaaaatggttcttctttttcgcgcagggatttggtttttcatttttcatttttcatattttatgttattttattttatttttttgttttattttattttattttttttttttaaataagactgggtgttttcgtctcccacccctgctgaggaactgtttgatatacagaagcctgagatacttgagattatgtgacaaagaaattgtaaacctgaatgtgactaaggcagaagagagcaggtgaggccacacaatactgtacacccacttccgatcgtcatcgaaatgagcgttgtgggtactggaacgacaaaccatagatcggatctgcatccgaataggctttactttgtacattggtaggagtttgaatgtttgaagatccggcagatgatcacctgctgcagggaataatttgaaacctgtccgtctgctttgacccaattgacgaggtacgtgatttgatgctacccaagcagtatgaatgcgacggaggtggctatccttaccttgtcatatgcaactcataacgggtagtccccatggagccacggctatttatttctaagggccaacagtgaaagattgtgagatcgtatacctcctgggtatgacgcaaccagggtgagcactggcattgctccaatccatatggtggaggtatgtctccgagaccatgacgcatgtagaccctgacctgtccctgatccagggatctggtttgtcacttggcgtattttaattaactgcgggtcagggtcttgccgtccagacccaatggatgaattgcctgcgtggccctgcagttccagcgatcggcacggcacggctatgacgtcggtgggtgttccgtttgatatttaaccatagaagacagccttgttatgctggttcctgccgcaaaataaaggtctatccttaccacgttaagtgtcaaaatgagtggggtaagctttgcccgtcaggccttactccaggctgctcttctttcgattgtctccgcaatcccggcgcccacggccttcgtcaacaatgtcgctccgcccgagcccatcatcaccccttcgccggttcaacatcgaccttctcgagtcgctggtcgaaacattctgagtgacgtcgattctgatattaacagcatcctttctggccttggatccgacctaccctcctgggtcgcatcgggtgtgcccaactacttccagggtttccctactggggatgcagtggtgagctccttgggcttgaacagcgctgagttggcagccttgcccaccaatgtcttgaatatcgacccatatgccaactggactagctctggctggaacgttcgcttccacggaaacgtctacaagcagcccaacacctccatctccgacctcaatgatttggctgatgttttcctcggcaatacaagcatctccgacctttccgaatccgagcagaaacaggctcggaatttaacagcggaaatattagtggtccagcaagctcacgtcgccgtaaacacaatccacctggagcctgcaccgagtcagggatccagcggacagtcaggcggcggtggatcctctaataccactggcggcacccaagacctcactctgccctacaacactacagttgaaggcgattttgacactttcgtaccgattagcagcaatggcttgacggcaggtaatgaaacttcggcgatacaacggcttaacgtccatgtggagggcgcgacaataggaaacagtactgcctatctcgtacctccgaccgggctcactgtcgtttcggacatcgatgatatcctacgcgtcaccaaaatctatgaaccagagcaaggtttactcaactcatttgcgcgccccttcaggccgtgggagaatatgccggacatctatcgcaactggtcgatcagtctccccaacctgcactttcactacctagtaagtctgctagatgtccctgattgcattgttgctgcatactcaccactacactacagacgacgacccccgaacaagtcaccaggaactacatgcaattcatctacgacaactaccccggtggatctttcgacacccgtcccctcaactttagcgacgtttccgccaccttgtcgatccggaaattcctcctgcaaaaggtcttcgagacctttccgcagcgcaagttcatcctcatcgccgacaccagcaacagcgacgtcatgcgtgactatcctgaaatggcgaccgacttccctggtcaagtgcaatgtatttttctccggaacaccagcgccaccgactcaggggacaaattcccatacgatacctctgggtttaagaagctcaatcagtcaaactacatgttcttcttgcatcccgatgacttgacgaatctagacatcgcgaatggccagtgttacaacacatccattccgcagaatttaacgtttagttatcagggtttgccattggggctgggtgatgaacccacagctgtcaatgggtctgccaatcatactgctgagtcggcggccggtttgtcggtaaagggtgggactgatatgcagggtttgatctttctgttcaccctggttacggcctattccattttcttgtaaatatcagttacgatgtatgagagataacgactttgatatactcacggctcaatttacgttccaaaaccgaatacagcggatatctatgctcgatgcaaggactaccactatgttacactactaagggtttatctactatgggaaatattagtgcttggtttgttacagctcactcgagcttgcgttactaaccacttttatcggtccatggatccaccgcccaacgcagttatcccaggaggtggccgcaatggccagtcaagtcaattaaatctcctctgctaggaattgcatgggttttgacagacagaactcggggaaagtatcgatattcaatgatctgacggtggctccgaaattcgctaggctagcccttcatcactagtaatattagttgtccaactttcgcgctttacactttccatagcaaacccccaattcccagccaaatcctcatatcctctagga

SEQ ID NO: 22 - DNA, 3771 bp, Aspergillus niger
gcttctatggtatcaagggtcgcgtagccaaaagtttaactgcatcgctgcggtattagtcggtgtagctggctgcagctttcgttgtgtccctggtctgtttaagaaacggtatcagatctgtcccgcccgtccccctgacaccggcattgagtttctcatgagaattgtccactattgtcgacgttgttgtggctaggtttgttctcgaagatacttggtttggcggcggagtccttgcagccatgatgatgtatcgagtcaccatccgcacatgcttatcgcgaaatcccccaagtgacataaccgccatgttatatgcatccctcaaagcagatccgggcttgtgacttaatacataggatctcacattagacgtgcggagcatcatttccaagaagcgtcggtgagggcctggcatgtagtttcgcatgttcttgaatatacagtcagtatcgacattgctttgtctggataactcctcgaagatcagtccttacctgcataaaaccagtcatgacttgaggattcgacttgccgtgaggcttggtctccccagtggccgaatgttccacacccaagaaaatgtcgaaggcttggatcagagaactttgagcattgctaccaccactgtattgatgccattcgcctcgtccttccccaacatcatagaatacgccgtttggcagcccggccgatgccatgttcttgctgccggctaggtatggacgtaattgatggtaaaagacgggcggcgcacacttttcatacatgcgttccaagaggtcaccgagttccttgagaccataattgagctgctccagacgagaaataacacgctggctatcatcaacattagctgcatgcatagcatccagcattagctcgatgagctgtgctcccttggcctcgatagcgacggagaggacgaaaaaccattcttcgtctttggtgcctgtgaacgaggtcaccactgataaattgtctggatccgtaacgtctgtatcagtggatggtactgaaaagttccaaagattgagggcagcatatgtagcacatggtggtagctctaagtggttcgaaacttcgatgaatgggcgtgatatacaggggggtagcacctgtatagttgatgtcagcgaaggcacgggccatgcagtgatagacaagaaaagagggattgaataaaagactcgattctgaaggggagtaatacatctttcggcttctcaccaccccagatgtaggcgtgtgtaaggtaagcaagcacaacatatgctctccgccactcaggttcccccttcaacttcttcgtagagagtatacccaggtcatcgacagacctcctgatagtaccagactgaatcaggccaggcaagtcctgagcgatttcctcccacggggaatagtaagggtcttccaatctccgtacgggaggaacttccgggaggaacccgttctggagggagacaccgtacgcaacaagtgaaatgtcttgtgtataaagcatgataatatatgcacttcagtagacaacttgatattcaagcctccttgaaatgatcctcatgcaggtaagagtagatccaagtgtttatattgtattcccaaggagggacggaagaaaaccggatccagagggctcgagatagggccacctgcatgagcaaagacgggtcatttcccgtttcacaacgaccattctcaggcagaaaggcagtggattagcggaaaacttagcaaccatccggatttctggagtggatgctttgttatctcacttccggcaacacggatggtgaggcggaagcggaacggtatccggatccggagggagggacaaaccgaagtgccttgtgtcacacgccggtccatatataaatgttaattggtgacggtcagcggattgtttctggtgttcatccattttttcttgatatcctggacccagttattgcactttaattactatccaaaatccacaatgtcgaagtcgaatggcgtcagcctggcattccccgccgaagcagcgacaaaggaatatgcagcatctctagactcttctgatagacttgctgcatttcgggagaagttcatcgtcccatcgaaagcaaatattgcttccaaaaagctagcaaagcctggtgggttccatcctggcaactgtagacctcgtcactaatgtcacaggcctttcgcccgagtcatgcatttacttctgcggtaactcacttggcattcaacccaaggccacagcaaagtacttagaagcacagttggacacttggtcatctattggagttggcggccacttcactgatcttgagggctcacccttgaagcaatggcagctgctctcagagcaagcagctgattcaatgagcaagattgtgggagcaaagccagaagaagtcgcagcgatgggaacacttacaacgaaccttcatcttctgctggctagtttctataaaccgactcaaacaaagcacaagatcttgatggattggaaggctttcccaagtgatcacgtaagttcgaaagaagctgccttctgctatcacggcactgacgattatatagtacgctatcgaatctcatattgcctggcatgacctggatcctaaggagtctatggtgctcatcggtccagatgaaggcgaatacgagatatccacccagaagattttctcttatatcgacaagcatgctgatgaggctgctatgattcttcttcctggtattcaatattatactgggcaactgttcgatatccagaaaattacaaaatatgcccattcgcgcaacatggttgttggctgggatctggcccatgcgttcgctaatgttgagctaaagctgcatgattggaacgttgattttgcagcatggtgcacatataagtacggaaacgccggtcctggagcaatgggggggcttttcgtgcatgaacaacacggagaggtcgattacagcgcgggagaagatgcacccaaattccgccatcgtctgaccgggtggtatggtggcgatcgatcggtaagattcaagatggacaacagtatgtgtgcacttggctatagaacaattttgtgggctagtaagctgatacgattgataaccagagttcaaacctattcctggggcaggaggatttcagatatcaaatccttcggccatcgaccttgcgtgtctttgtgccgctttatcggtgtttgatgagacgtcaatggcagatcttcgcagaaaatcattgaagctaacagcataccttgagttccttttgctcagggattatgaagaagaatctaggccattcagcattatcacgcccaaggaccccgaggcgagaggcgcacaactgagtctattgctcaagcctggtctattacagaatgttgcacagaagttgcaagaagcaggaatagtctgcgataaacgggagccgggtgttgtgcgtgtcgctccggttcctttgtacaacagcttcagcgaggtttggacatttgtgaagatcttcaaggatgcactgcagcagtgatgggtctcactagatcatttggtcaggactgagaccagatttgtcaagttcgctccgatgaatgattttttagatagataccaaattatttgattcttagatttgttgaagctctcactgcgacatggtccacagccactgtgcaaaagcaaataactgaagtaggaactctatgatagacccgtactgttgaggttatagatttccaaagccgttccttggatcaccacttc

SEQ ID NO: 23 - DNA, 3562 bp, Aspergillus niger
cctgtgcttgtggtatgacagagccactgatatcatctcctctcatctccagttgtaatcggaattgatcgagggggccaatcttccacacagccccatggaagacgcacagatgctttcgatgccacgctggcagacggccgccaactgtcatcgtccagtgctgccgccattcccatgcaaaagccaacgctaagaagcgcgcgggcgccgagtttccacgcttgcagcttagttcagcgacccagctcagtggcggctcgctcatgcatagaaagatgggtttggacccgcgctgggcctttcatctgcaacactgctcgcagaccatgtgtgacttttggctgcaaaaaaaaagaggctggcgacagctgatccactgtgctattcctatctcatcctccccggctggatcgaaagctgcacggcaggggaaatcaacagttgggctctgacacgtatcgagctaactgatgcgctgtccttccgcctaatgcctactaagtcttagggacgggaccatcatccaacatgccaccttgtccttcgtaattcctttttgatctcggtgatggtggccacccaggaaatgacgttcctcaccttggctgcaattggtgcccgatgtctcatctccgctcagaacggcggcgagggcagaataaaaattgacgggcacagcaggaactaatcaaacataccggcggtcttctaaatgatcgtatctcccggaatactccctctttctacagaagcggggatccattcgccctatatgtatagtggagctttgccgcactgattcgcacggcattatacctccgcactgccgtgccttcatccacttgattgtggccagggaaatatcaacggggcagataacttgcccttgcatcattattgctccgcacattcattcattatcgctctgatcccacatcctcacgcagctgcgaccggagtgtccacactctaattgggatgagcattgcctttcaggaacttgactttttgtggttgcatcttttgttgccttacccacgcaggcttcgtttatgagatagcgatttcaggcctcaaattgctgtcatcacttccggccccaaactcgtgggcggtgcccgcccatcgctggcaagaagcacagactaacgccttacttcgcctagttgcacacgatgcatcaagacttcctcctatttcacaacgaccagctccttgccatttgttgtttcgaatgcccccttccccgtgccttgcatttctaattcccatctatacccttttgcgctctcacgcccgcctgactatcaatttttctgccagcataaccctgcgtgcagagcggttgacagcaggcttgctaagagtcttcgtctagaagaactgtctgggtggattccttttcggattaacttttgctattttaaaagctgattgcactacggagtacacccgcaatctgattctcactgatactccaattaaaggccactaattgctgacatttgtaacatatttgcgtttgctttccggttgaaattggcggctttgtgctttcgatcgctagcagtctctgtgcatgtacaactgatcggctattcagtcctattcggaagcggcactgagaggcggagacaagccacagatacgacgagtctgattttgagacgggcgcatccaacgaaagcctgcgattgcaattgaagatctcaattgggggactgacaacctaatacctaaacatatcgaggattaggcgccaacacgagcatgggaagctttggtgcatgcgacggctgatccatttctaacaaccgttttgcgcgcgcgtccgaggtaaggctcccgatcccttggcgctttgcatgtttcactccgatgcatctactgtttatatgagtgcggtgggcaagctgtgcccaccgactgagcattctcaaccggcccaatgctgtgggatcgatcctcccctccaaagttgtcgctcggttacacttgatcggagcttgttctgttatacaccaaaccatcatggatgtgactaatttggctgtcgattgtcccgttatagagaccggttcccttgctttgtggatggcttcctccgaagtggaccagaaggccttgggcaactttgcggccataacgggcctcgagaacccgttcctgtccgcgatgctgtacaagattctggggttgtctgttatgttatcgaagaaactcctccgtgcacgacgattgcggcgcctagaccccacccgagagactaaatcgcttcatctttattaccatatcatctggctttcgcgtgagggtcttctgatattagaggagttcgtcctgccactggttgagggatttatggaactcaaaattttagcttacaagctgcgcgcctccttttaccatatattcgtcctatttcagaaccagcctgctgtccactcgccgggtatcgttagtctgccgagtagtacacctttatccaacggcgtgacagaagcggagtcaccatccaaggattccaatttgagattttcatttcagctgaagcccgacacgataacggtttccgggaagcccagttccgcctcggatagtgcgccacgaggcaaggttacgcaggcacccccgggctttgcgccggttcaaccgcccaagtcgaactcggcgtttctcctccctgctctcgattatacccctacagctaccgcatgcttcaatcatgccgccctcttggcagatcaattcctcccggggtcgcacccgctccgtctgtccatcaagcttgaatttgccgcctacctctacgattgcctacatgacgctcatgcctgtcgacggttggccaagcaagccattgccgatgtgtacaatgcgcaagagggcatggacgacgagagcttcgaggatgcagctgagattgtgggtattttgggcaagatggtgaagcggggaaggaacacaagcagtgctggaaatagcaccacggctgtgaaaacgccccaggaggagcgaagtgagggcagtcgaacaccatcctctcagacaacgttgaaaaggccggcgcgagtgccgaaaagcacctcctcaccggcgcgcgcgaccaagccgacgactagcgaggggatgtcccccgctgtgcccgaccccaccatgatgaatcccatatgatgcctttccctacgacctgtatatgcttcttcttggaactatggagtcgcgcatgagcgcttggcctgtgacttctacttgacttgcacctgtctgtatcatctcggtaacggagttgcggcgatagactggaattgggcatcagcttgcggggagctgtggaatgagcgattaagcgcgacaaaaacttgaggtggaagggacagaccagccaatttgtgatggtgactgtagatagatagatgtgtgcaatacaaagtatgttgttgatgtcatgcaccccacttgtgtatgcttgtctggacaatggacctacacttagtcgcaacaactgtatgtctcaagccaatggccatggggcaggatggatcgatggatctcttgt

SEQ ID NO: 24 - DNA, 4361 bp, Aspergillus niger
cgacggactaacgtcgcgcgccccatttcacgtcttgaagacagatgtgtatgcgattgctcgcgaaattctcatccgattccgtcggcttgacctgacatctggaatagacgaacaggacttggaagctatcattcgtattaccgaagagtccaacgtacttctcactgactggtggaagtccgtcccgaaactgtatgacttcgattactggaaagcagacggtcgatgggaaatgattgaaggccagctgcaaaggcttccctccgaagcaaggcaacaggtggagacaatatatctacaggctgctgttctccagttgacgtacgacggcatggtcatccaagcaaatagaccactgttggagcgaagaatcaacaggctaacatcttcgcgggcgatcgtggatgcgatgcatagggcgcttgaccgagccactgctgctgctctccgcatatcccgtgttccagttcacaagctgaaaaatcattttgctatagcagtcgtatcgatgcaagagttcactgctggtgtgattctctgtatacctcccacggtgaagcctttaacggccaccgctcatgaggcgaaggacggggtggtaagaataatacgcgccagtcgaagtctcaagagccacgatcgaatagcaaggcaaactgaacaacttctgacggaactactcaaagttaccacccaacgcgagataacgcacgccttggccgataatctcgacaccaatttcccctccgatacgcggctaacaggctccgttgttgccggcgcctcaagtgactccttgggtgcaggacgctccaggccagttcccggatccgcagcgcaggcagttgtagatcctatccaaggagcctgggactcagaattcaatgccctccctgccgtaacgggaccttatgatttcctccctgctcaagcctttcaacagctggacgatacatttggtgcctttggagaatgtaatatcccttgatcaccccagactcacatccagattctgactgacctcgtgcatagccatatttaacttggtccctgatgatcaaaacagtacatggaactggggccgaacatttccctagccaaaggagcatcagggattcaacgtgatcagtacagccttgtagctcgctgaaactgccctggagccaagccaaagcctagacagcaaggtgcatgggcgagctttgcgaggatttagttgttatttagtatgccaagattaagccacgcagaatggcttatggccatagtagcaacaacggccacggaacgttcttccatgtggtatggggaaagccgccggatatttggggagggcgtgattgttccggtggctatctttccggtggagataacggcggtggaccatcaaaactcctcagcccggattgctcggactccgcatcgtcaagtatcggtttcatgacagccattcgactacttagaagacacaaatagctatccaacttacccggacctttgataataattttcagcagatctgtcgatctcagagacaataatgccaccgttctgtgtggaaggcactgcgccgtggtttaacaataataaaactccaaattgcccaaaagctagagcgaaggaaagtcgagaagcccagtctagactggcgggctgatcgtccggttcacaacctgattgacaaatcatctgtgaccctcgatgttccgcggcgttgagcgggtgagaatgtggatcctagtggggaagacccaattgccgccggaaaaggtgagcttgtgctgcacatgtccggtggctcaattcagggccatgccaggacattgctcttcaaagggatgcgattgctattccctatttacttctttaacccgctgacgggcctgtctggaccgagaattcttggtcaagtttcagtggagcgatccaggaaagaacatccgatatggacagttgccctcgatctcaggtctgtagtcgtgtgacacagaggcctctactcgcttgccttgcatgtcggagctcaaaggtgcgatgtgaattaacacgaggagcgactgtctgtcgcagatgcaacatgagaaatttggagtgtgtctacacaaaatcacgaaggtatccctcgtctatctttccgtgtgtattattagccgagagtcacgcaatgctctttgtatacaatgaaataatgttggagctagagaatgatttctgatagttatatgttacaggacactcaggaggtcgtcaactagtccgaacacggcgtcctctcctgacactaatgcctcatcttcgccatgctcggactctagtgggacccatgctggcatttccattccccactcagatagtcccgccagtgtggacgaatgtgggaacaacggccttttcgacgggggtaggtaatatgtcatgcttacctgcgacactcactcaccattcttctagaatctctaactcaattattggtctctgggagcaaagccatatcaggccaaccacatatcgccattaagccacatgttctgtctacctatccccttgacaacctcctctcagacattgagggactcctcgagcagccagtctctcatttggctcaaagcaaggaagatctaacaaaaccggaccggatgtttcgaactcgccttccagcggtctcaggtgaggacaatgagtatctgctaaggaaaggagctctgctgctcttgccaccagccttgcgcaacgaactattggcggcatatgtcaactacgtccatccgctattaccgattcttgatcttggagactttctctcacaaatcattgttggagatgatgctcaattcaagtcaccactattataccaagctgtcatgttcgcgggcagtatttttgctcagcgagacggcacagacgaacctgaacagctaacgagggctttatttgaacgtacaaaggttagtaatggttacaattagacatatcttagatatactgagcgatttccaggttctatacgagtttgaatccgaatcatgtgcctacactcaaattcaagctctcttattgatgactctctggcatggagatatgtcacgccacaaaggcccgtcttactggcttgacgtcgccttctccactgcggaaagaattggcctactccatgatgaagactggccatgcgatagccgttccttccgatccgctatgtggtgttgcctttacgtacgtgatcgaacaatctcgcttgggtctcgtcggcctccacggatgccagtcaacaaatatccggactcaattctccatcttatgagcgacgcacctcgggaatatgacgaaataattcttgagagtctaggtagcgcttctctgatgcttacaagcacttaccaagaacaatcgaccatgctcttcaaggaacttgtcaaactctcctactgcgtcggggcaatcttagattatctatacgaaggggcctgggtccgaataccaacacgtcgcagcagcttttattcactcgcccctaaatgcagcatctcgccatccatcagaccaagctgtgagaaactactttactcttggattcaattcctccctctcaacgcagtctaccatccgccacagttgcccgccgatgcggaaggggagtcagccgaaacagtcttccttgtccacaaagccttcctttacttgctgtacctagcctccatggcagtcatataccgcacaggcactcatagccaacaaaccagcacggcaactccgaacatggaaggtctacgggcatcgacgtcacaaataaaaaaggtccttgcggagctgcagggcatgggactgcttcagttccttccaggggcatcagtcactatcctgatgttcgctgtggaggctagtctgttggatctccagggctcggataccatggtccgaaggcaggctatgtcaaatctatatgcctgtgaggaggctgcgttacacttgatgcaggcttatcctacggctgagatggcggtattgaagactaggacagcccgtgcaaaccttcttgggttggttgagacatgaagaccaatcatggaagctctgcttccatcaaagcattgtgtctttgcttaatgaatgaatggattatgagcgtttcgcaaccttctttaatgcagctacataatagagcataaacagaggcccggaaatatataatttggctaagaacccggttgcaagcaggcaagtcacgaggtgcagaacataccatcaagtttatagattattgtaggggatatcctcaacaatatcttacttccaccattacgtactgaagatcgaggttatcggggaccatgctgtcgtgtcaataggtacgactcgtt

SEQ ID NO: 25 - DNA, 801 bp, Aspergillus niger
  CDS (1)..(801)
atgcctatctccattccctccgcctccagcgtccacgacctcttcagcctgaagggcaaggtcgtcgttatcaccggcgcttccggccctcgcggcatgggcattgaagccgcgcgtggctgcgccgaaatgggcgccaacatcgccctcacctactcctctcgtcctcagggtggtgagaagaacgccgaagaacttcgcaacacctacggcgtcaaggccaaggcctaccagtgcaacgtgggcgactggaacagcgtcaagaagctcgtggacgacgtgctggccgagtttggccagattgacgccttcattgccaacgctggcaagacagccagcagtggtatcctggatggttccgttgaggactgggaagaggtcatccagacagacctgacgggtaccttccactgcgccaaggcggtgggcccgcacttcaagcagcgcggaacgggcagcttcatcatcacctccagcatgtcgggtcacattgccaacttcccgcaggagcagacgtcgtacaacgttgccaaggcaggctgcatccatatggcccggtcattggccaacgagtggagggactttgcccgcgtgaacagcatctccccgggttacatcgacacggggctgtcggactttgtggataagaagactcaggatctgtggatgtcgatgatccccatgggacgcaacggtgatgccaaggagctgaagggtgcatatgtctatctcgccagtgatgccagcacatacacgacgggtgccgatttggtcattgacggaggatacaccgtgcggtaa

SEQ ID NO: 26 - DNA, 1602 bp, Aspergillus niger
  CDS (1)..(1602)
atgagtggggtaagctttgcccgtcaggccttactccaggctgctcttctttcgattgtctccgcaatcccggcgcccacggccttcgtcaacaatgtcgctccgcccgagcccatcatcaccccttcgccggttcaacatcgaccttctcgagtcgctggtcgaaacattctgagtgacgtcgattctgatattaacagcatcctttctggccttggatccgacctaccctcctgggtcgcatcgggtgtgcccaactacttccagggtttccctactggggatgcagtggtgagctccttgggcttgaacagcgctgagttggcagccttgcccaccaatgtcttgaatatcgacccatatgccaactggactagctctggctggaacgttcgcttccacggaaacgtctacaagcagcccaacacctccatctccgacctcaatgatttggctgatgttttcctcggcaatacaagcatctccgacctttccgaatccgagcagaaacaggctcggaatttaacagcggaaatattagtggtccagcaagctcacgtcgccgtaaacacaatccacctggagcctgcaccgagtcagggatccagcggacagtcaggcggcggtggatcctctaataccactggcggcacccaagacctcactctgccctacaacactacagttgaaggcgattttgacactttcgtaccgattagcagcaatggcttgacggcaggtaatgaaacttcggcgatacaacggcttaacgtccatgtggagggcgcgacaataggaaacagtactgcctatctcgtacctccgaccgggctcactgtcgtttcggacatcgatgatatcctacgcgtcaccaaaatctatgaaccagagcaaggtttactcaactcatttgcgcgccccttcaggccgtgggagaatatgccggacatctatcgcaactggtcgatcagtctccccaacctgcactttcactacctaacgacgacccccgaacaagtcaccaggaactacatgcaattcatctacgacaactaccccggtggatctttcgacacccgtcccctcaactttagcgacgtttccgccaccttgtcgatccggaaattcctcctgcaaaaggtcttcgagacctttccgcagcgcaagttcatcctcatcgccgacaccagcaacagcgacgtcatgcgtgactatcctgaaatggcgaccgacttccctggtcaagtgcaatgtatttttctccggaacaccagcgccaccgactcaggggacaaattcccatacgatacctctgggtttaagaagctcaatcagtcaaactacatgttcttcttgcatcccgatgacttgacgaatctagacatcgcgaatggccagtgttacaacacatccattccgcagaatttaacgtttagttatcagggtttgccattggggctgggtgatgaacccacagctgtcaatgggtctgccaatcatactgctgagtcggcggccggtttgtcggtaaagggtgggactgatatgcagggtttgatctttctgttcaccctggttacggcctattccattttcttgtaa

SEQ ID NO: 27 - DNA, 1392 bp, Aspergillus niger
  CDS (1)..(1392)
atgtcgaagtcgaatggcgtcagcctggcattccccgccgaagcagcgacaaaggaatatgcagcatctctagactcttctgatagacttgctgcatttcgggagaagttcatcgtcccatcgaaagcaaatattgcttccaaaaagctagcaaagcctggcctttcgcccgagtcatgcatttacttctgcggtaactcacttggcattcaacccaaggccacagcaaagtacttagaagcacagttggacacttggtcatctattggagttggcggccacttcactgatcttgagggctcacccttgaagcaatggcagctgctctcagagcaagcagctgattcaatgagcaagattgtgggagcaaagccagaagaagtcgcagcgatgggaacacttacaacgaaccttcatcttctgctggctagtttctataaaccgactcaaacaaagcacaagatcttgatggattggaaggctttcccaagtgatcactacgctatcgaatctcatattgcctggcatgacctggatcctaaggagtctatggtgctcatcggtccagatgaaggcgaatacgagatatccacccagaagattttctcttatatcgacaagcatgctgatgaggctgctatgattcttcttcctggtattcaatattatactgggcaactgttcgatatccagaaaattacaaaatatgcccattcgcgcaacatggttgttggctgggatctggcccatgcgttcgctaatgttgagctaaagctgcatgattggaacgttgattttgcagcatggtgcacatataagtacggaaacgccggtcctggagcaatgggggggcttttcgtgcatgaacaacacggagaggtcgattacagcgcgggagaagatgcacccaaattccgccatcgtctgaccgggtggtatggtggcgatcgatcggtaagattcaagatggacaacaagttcaaacctattcctggggcaggaggatttcagatatcaaatccttcggccatcgaccttgcgtgtctttgtgccgctttatcggtgtttgatgagacgtcaatggcagatcttcgcagaaaatcattgaagctaacagcataccttgagttccttttgctcagggattatgaagaagaatctaggccattcagcattatcacgcccaaggaccccgaggcgagaggcgcacaactgagtctattgctcaagcctggtctattacagaatgttgcacagaagttgcaagaagcaggaatagtctgcgataaacgggagccgggtgttgtgcgtgtcgctccggttcctttgtacaacagcttcagcgaggtttggacatttgtgaagatcttcaaggatgcactgcagcagtga

SEQ ID NO: 28 - DNA, 1143 bp, Aspergillus niger
  CDS (1)..(1143)
atggatgtgactaatttggctgtcgattgtcccgttatagagaccggttcccttgctttgtggatggcttcctccgaagtggaccagaaggccttgggcaactttgcggccataacgggcctcgagaacccgttcctgtccgcgatgctgtacaagattctggggttgtctgttatgttatcgaagaaactcctccgtgcacgacgattgcggcgcctagaccccacccgagagactaaatcgcttcatctttattaccatatcatctggctttcgcgtgagggtcttctgatattagaggagttcgtcctgccactggttgagggatttatggaactcaaaattttagcttacaagctgcgcgcctccttttaccatatattcgtcctatttcagaaccagcctgctgtccactcgccgggtatcgttagtctgccgagtagtacacctttatccaacggcgtgacagaagcggagtcaccatccaaggattccaatttgagattttcatttcagctgaagcccgacacgataacggtttccgggaagcccagttccgcctcggatagtgcgccacgaggcaaggttacgcaggcacccccgggctttgcgccggttcaaccgcccaagtcgaactcggcgtttctcctccctgctctcgattatacccctacagctaccgcatgcttcaatcatgccgccctcttggcagatcaattcctcccggggtcgcacccgctccgtctgtccatcaagcttgaatttgccgcctacctctacgattgcctacatgacgctcatgcctgtcgacggttggccaagcaagccattgccgatgtgtacaatgcgcaagagggcatggacgacgagagcttcgaggatgcagctgagattgtgggtattttgggcaagatggtgaagcggggaaggaacacaagcagtgctggaaatagcaccacggctgtgaaaacgccccaggaggagcgaagtgagggcagtcgaacaccatcctctcagacaacgttgaaaaggccggcgcgagtgccgaaaagcacctcctcaccggcgcgcgcgaccaagccgacgactagcgaggggatgtcccccgctgtgcccgaccccaccatgatgaatcccatatga

SEQ ID NO: 29 - DNA, 1779 bp, Aspergillus niger
  CDS (1)..(1779)
atgagaaatttggagtgtgtctacacaaaatcacgaaggtatccctcgtctatctttccgacactcaggaggtcgtcaactagtccgaacacggcgtcctctcctgacactaatgcctcatcttcgccatgctcggactctagtgggacccatgctggcatttccattccccactcagatagtcccgccagtgtggacgaatgtgggaacaacggccttttcgacggggaatctctaactcaattattggtctctgggagcaaagccatatcaggccaaccacatatcgccattaagccacatgttctgtctacctatccccttgacaacctcctctcagacattgagggactcctcgagcagccagtctctcatttggctcaaagcaaggaagatctaacaaaaccggaccggatgtttcgaactcgccttccagcggtctcaggtgaggacaatgagtatctgctaaggaaaggagctctgctgctcttgccaccagccttgcgcaacgaactattggcggcatatgtcaactacgtccatccgctattaccgattcttgatcttggagactttctctcacaaatcattgttggagatgatgctcaattcaagtcaccactattataccaagctgtcatgttcgcgggcagtatttttgctcagcgagacggcacagacgaacctgaacagctaacgagggctttatttgaacgtacaaaggttctatacgagtttgaatccgaatcatgtgcctacactcaaattcaagctctcttattgatgactctctggcatggagatatgtcacgccacaaaggcccgtcttactggcttgacgtcgccttctccactgcggaaagaattggcctactccatgatgaagactggccatgcgatagccgttccttccgatccgctatgtggtgttgcctttacgtacgtgatcgaacaatctcgcttgggtctcgtcggcctccacggatgccagtcaacaaatatccggactcaattctccatcttatgagcgacgcacctcgggaatatgacgaaataattcttgagagtctaggtagcgcttctctgatgcttacaagcacttaccaagaacaatcgaccatgctcttcaaggaacttgtcaaactctcctactgcgtcggggcaatcttagattatctatacgaaggggcctgggtccgaataccaacacgtcgcagcagcttttattcactcgcccctaaatgcagcatctcgccatccatcagaccaagctgtgagaaactactttactcttggattcaattcctccctctcaacgcagtctaccatccgccacagttgcccgccgatgcggaaggggagtcagccgaaacagtcttccttgtccacaaagccttcctttacttgctgtacctagcctccatggcagtcatataccgcacaggcactcatagccaacaaaccagcacggcaactccgaacatggaaggtctacgggcatcgacgtcacaaataaaaaaggtccttgcggagctgcagggcatgggactgcttcagttccttccaggggcatcagtcactatcctgatgttcgctgtggaggctagtctgttggatctccagggctcggataccatggtccgaaggcaggctatgtcaaatctatatgcctgtgaggaggctgcgttacacttgatgcaggcttatcctacggctgagatggcggtattgaagactaggacagcccgtgcaaaccttcttgggttggttgagacatga

SEQ ID NO: 30 - protein, 266 aa, Aspergillus niger
MPISIPSASSVHDLFSLKGKVVVITGASGPRGMGIEAARGCAEMGANIALTYSSRPQGGEKNAEELRNTYGVKAKAYQCNVGDWNSVKKLVDDVLAEFGQIDAFIANAGKTASSGILDGSVEDWEEVIQTDLTGTFHCAKAVGPHFKQRGTGSFIITSSMSGHIANFPQEQTSYNVAKAGCIHMARSLANEWRDFARVNSISPGYIDTGLSDFVDKKTQDLWMSMIPMGRNGDAKELKGAYVYLASDASTYTTGADLVIDGGYTVR

SEQ ID NO: 31 - protein, 533 aa, Aspergillus niger
MSGVSFARQALLQAALLSIVSAIPAPTAFVNNVAPPEPIITPSPVQHRPSRVAGRNILSDVDSDINSILSGLGSDLPSWVASGVPNYFQGFPTGDAVVSSLGLNSAELAALPTNVLNIDPYANWTSSGWNVRFHGNVYKQPNTSISDLNDLADVFLGNTSISDLSESEQKQARNLTAEILVVQQAHVAVNTIHLEPAPSQGSSGQSGGGGSSNTTGGTQDLTLPYNTTVEGDFDTFVPISSNGLTAGNETSAIQRLNVHVEGATIGNSTAYLVPPTGLTVVSDIDDILRVTKIYEPEQGLLNSFARPFRPWENMPDIYRNWSISLPNLHFHYLTTTPEQVTRNYMQFIYDNYPGGSFDTRPLNFSDVSATLSIRKFLLQKVFETFPQRKFILIADTSNSDVMRDYPEMATDFPGQVQCIFLRNTSATDSGDKFPYDTSGFKKLNQSNYMFFLHPDDLTNLDIANGQCYNTSIPQNLTFSYQGLPLGLGDEPTAVNGSANHTAESAAGLSVKGGTDMQGLIFLFTLVTAYSIFL

SEQ ID NO: 32 - protein, 463 aa, Aspergillus niger
MSKSNGVSLAFPAEAATKEYAASLDSSDRLAAFREKFIVPSKANIASKKLAKPGLSPESCIYFCGNSLGIQPKATAKYLEAQLDTWSSIGVGGHFTDLEGSPLKQWQLLSEQAADSMSKIVGAKPEEVAAMGTLTTNLHLLLASFYKPTQTKHKILMDWKAFPSDHYAIESHIAWHDLDPKESMVLIGPDEGEYEISTQKIFSYIDKHADEAAMILLPGIQYYTGQLFDIQKITKYAHSRNMVVGWDLAHAFANVELKLHDWNVDFAAWCTYKYGNAGPGAMGGLFVHEQHGEVDYSAGEDAPKFRHRLTGWYGGDRSVRFKMDNKFKPIPGAGGFQISNPSAIDLACLCAALSVFDETSMADLRRKSLKLTAYLEFLLLRDYEEESRPFSIITPKDPEARGAQLSLLLKPGLLQNVAQKLQEAGIVCDKREPGVVRVAPVPLYNSFSEVWTFVKIFKDALQQ

SEQ ID NO: 33 - protein, 380 aa, Aspergillus niger
MDVTNLAVDCPVIETGSLALWMASSEVDQKALGNFAAITGLENPFLSAMLYKILGLSVMLSKKLLRARRLRRLDPTRETKSLHLYYHIIWLSREGLLILEEFVLPLVEGFMELKILAYKLRASFYHIFVLFQNQPAVHSPGIVSLPSSTPLSNGVTEAESPSKDSNLRFSFQLKPDTITVSGKPSSASDSAPRGKVTQAPPGFAPVQPPKSNSAFLLPALDYTPTATACFNHAALLADQFLPGSHPLRLSIKLEFAAYLYDCLHDAHACRRLAKQAIADVYNAQEGMDDESFEDAAEIVGILGKMVKRGRNTSSAGNSTTAVKTPQEERSEGSRTPSSQTTLKRPARVPKSTSSPARATKPTTSEGMSPAVPDPTMMNPI

SEQ ID NO: 34 - protein, 592 aa, Aspergillus niger
MRNLECVYTKSRRYPSSIFPTLRRSSTSPNTASSPDTNASSSPCSDSSGTHAGISIPHSDSPASVDECGNNGLFDGESLTQLLVSGSKAISGQPHIAIKPHVLSTYPLDNLLSDIEGLLEQPVSHLAQSKEDLTKPDRMFRTRLPAVSGEDNEYLLRKGALLLLPPALRNELLAAYVNYVHPLLPILDLGDFLSQIIVGDDAQFKSPLLYQAVMFAGSIFAQRDGTDEPEQLTRALFERTKVLYEFESESCAYTQIQALLLMTLWHGDMSRHKGPSYWLDVAFSTAERIGLLHDEDWPCDSRSFRSAMWCCLYVRDRTISLGSRRPPRMPVNKYPDSILHLMSDAPREYDEIILESLGSASLMLTSTYQEQSTMLFKELVKLSYCVGAILDYLYEGAWVRIPTRRSSFYSLAPKCSISPSIRPSCEKLLYSWIQFLPLNAVYHPPQLPADAEGESAETVFLVHKAFLYLLYLASMAVIYRTGTHSQQTSTATPNMEGLRASTSQIKKVLAELQGMGLLQFLPGASVTILMFAVEASLLDLQGSDTMVRRQAMSNLYACEEAALHLMQAYPTAEMAVLKTRTARANLLGLVET

SEQ ID NO: 35 - DNA, 1076 bp, Artificial Sequence
  PCR product
  PRIMER_BIND (1)..(35)
  PROMOTER (13)..(1062)
  PRIMER_BIND (1037)..(1076)
acttcactcgagataatgtaggcgacaaagtagccgggcatgcgaccggaggactcggtcagaggcacgaatatgagaggccagaacgccgcacccatgttccaagatgttgtggcccagtaaaggttaggaaatggggtgttttgaacgtggaatcgttcttccatccagttgttgccggagccgtaggaaccggccgggataccagtgaggatggaactatacctattagcactgccatcttcgagaacaaggctaagacatacatgaaacaaacggtgaccgtgagaagccatttataagaggtgctccaattgaacctgaatatgttaaaactagtatactagcaactgcatgcttgacgtacggattgtccgggtcatccttgccgttccaggtatggatctcaaggtcctccctgtcacgcagatcattgtggctgactaccccgacatgggggtcccggcgaaagaacgaccgcaccctgcaacgtcacttagtggctgtctgacagggattgattggcaatgattggcagactaaccgtcccagaaacgaatcatcgaactcattggtgtgcactgtgctagcgcggcgcagcgacgggcgtggctcctcggcaggggtggacatgactttctctgtgtagattccttgcaaggtatctgcttgacagatgcagagatgatgttgagtaaaatagggtgcagagaccctgcagaacctgcatcggccggcggcgacatgtcgtcatgaccgacccgggccgagcggaattaatgaaactcggaacagccaggggaatccgacgctttgagtggccaatccatgtctatccacatctgcattgaagtggatgaaaggtggaggagggctggttgccaaattctgttagggcttggaatgtaagcaaccggtttcggtcatgacatcatcacagcgatgacttcatcactctcggacgagcatatatagttgtgcctgtcgtcgtatctcaacaaacatcaacaacaacaacaatcatctactgcatttaccaaactcatctctacctcaatcaacttaattaatgaagt

SEQ ID NO: 36 - DNA, 1057 bp, Artificial Sequence
  PCR product
  PRIMER_BIND (1)..(33)
  PROMOTER (13)..(1043)
  PRIMER_BIND (1022)..(1057)
acttcactcgagttctcacaagcaaatccgagaaacgcaattgaccatatgtccttagtatgagggctagcaggaatacttgtttgtcgatgcataccgactaataaaaatggttcttctttttcgcgcagggatttggtttttcatttttcatttttcatattttatgttattttattttatttttttgttttattttattttattttttttttttaaataagactgggtgttttcgtctcccacccctgctgaggaactgtttgatatacagaagcctgagatacttgagattatgtgacaaagaaattgtaaacctgaatgtgactaaggcagaagagagcaggtgaggccacacaatactgtacacccacttccgatcgtcatcgaaatgagcgttgtgggtactggaacgacaaaccatagatcggatctgcatccgaataggctttactttgtacattggtaggagtttgaatgtttgaagatccggcagatgatcacctgctgcagggaataatttgaaacctgtccgtctgctttgacccaattgacgaggtacgtgatttgatgctacccaagcagtatgaatgcgacggaggtggctatccttaccttgtcatatgcaactcataacgggtagtccccatggagccacggctatttatttctaagggccaacagtgaaagattgtgagatcgtatacctcctgggtatgacgcaaccagggtgagcactggcattgctccaatccatatggtggaggtatgtctccgagaccatgacgcatgtagaccctgacctgtccctgatccagggatctggtttgtcacttggcgtattttaattaactgcgggtcagggtcttgccgtccagacccaatggatgaattgcctgcgtggccctgcagttccagcgatcggcacggcacggctatgacgtcggtgggtgttccgtttgatatttaaccatagaagacagccttgttatgctggttcctgccgcaaaataaaggtctatccttaccacgttaagtgtcaaattaattaagaagt

SEQ ID NO: 37 - DNA, 1061 bp, Artificial Sequence
  PCR product
  PRIMER_BIND (1)..(33)
  PROMOTER (13)..(1047)
  PRIMER_BIND (1023)..(1061)
atcctactcgagtggtgcctgtgaacgaggtcaccactgataaattgtctggatccgtaacgtctgtatcagtggatggtactgaaaagttccaaagattgagggcagcatatgtagcacatggtggtagctctaagtggttcgaaacttcgatgaatgggcgtgatatacaggggggtagcacctgtatagttgatgtcagcgaaggcacgggccatgcagtgatagacaagaaaagagggattgaataaaagactcgattctgaaggggagtaatacatctttcggcttctcaccaccccagatgtaggcgtgtgtaaggtaagcaagcacaacatatgctctccgccactcaggttcccccttcaacttcttcgtagagagtatacccaggtcatcgacagacctcctgatagtaccagactgaatcaggccaggcaagtcctgagcgatttcctcccacggggaatagtaagggtcttccaatctccgtacgggaggaacttccgggaggaacccgttctggagggagacaccgtacgcaacaagtgaaatgtcttgtgtataaagcatgataatatatgcacttcagtagacaacttgatattcaagcctccttgaaatgatcctcatgcaggtaagagtagatccaagtgtttatattgtattcccaaggagggacggaagaaaaccggatccagagggctcgagatagggccacctgcatgagcaaagacgggtcatttcccgtttcacaacgaccattctcaggcagaaaggcagtggattagcggaaaacttagcaaccatccggatttctggagtggatgctttgttatctcacttccggcaacacggatggtgaggcggaagcggaacggtatccggatccggagggagggacaaaccgaagtgccttgtgtcacacgccggtccatatataaatgttaattggtgacggtcagcggattgtttctggtgttcatccattttttcttgatatcctggacccagttattgcactttaattactatccaaaatccacattaattaatgaagt

SEQ ID NO: 38 - DNA, 1097 bp, Artificial Sequence
  PCR product
  PRIMER_BIND (1)..(34)
  PROMOTER (13)..(1083)
  PRIMER_BIND (1060)..(1097)
acttcactcgaggtccacactctaattgggatgagcattgcctttcaggaacttgactttttgtggttgcatcttttgttgccttacccacgcaggcttcgtttatgagatagcgatttcaggcctcaaattgctgtcatcacttccggccccaaactcgtgggcggtgcccgcccatcgctggcaagaagcacagactaacgccttacttcgcctagttgcacacgatgcatcaagacttcctcctatttcacaacgaccagctccttgccatttgttgtttcgaatgcccccttccccgtgccttgcatttctaattcccatctatacccttttgcgctctcacgcccgcctgactatcaatttttctgccagcataaccctgcgtgcagagcggttgacagcaggcttgctaagagtcttcgtctagaagaactgtctgggtggattccttttcggattaacttttgctattttaaaagctgattgcactacggagtacacccgcaatctgattctcactgatactccaattaaaggccactaattgctgacatttgtaacatatttgcgtttgctttccggttgaaattggcggctttgtgctttcgatcgctagcagtctctgtgcatgtacaactgatcggctattcagtcctattcggaagcggcactgagaggcggagacaagccacagatacgacgagtctgattttgagacgggcgcatccaacgaaagcctgcgattgcaattgaagatctcaattgggggactgacaacctaatacctaaacatatcgaggattaggcgccaacacgagcatgggaagctttggtgcatgcgacggctgatccatttctaacaaccgttttgcgcgcgcgtccgaggtaaggctcccgatcccttggcgctttgcatgtttcactccgatgcatctactgtttatatgagtgcggtgggcaagctgtgcccaccgactgagcattctcaaccggcccaatgctgtgggatcgatcctcccctccaaagttgtcgctcggttacacttgatcggagcttgttctgttatacaccaaaccatcttaattaataggat

SEQ ID NO: 39 - DNA, 1142 bp, Artificial Sequence
  PCR product
  PRIMER_BIND (1)..(35)
  PROMOTER (13)..(1128)
  PRIMER_BIND (1118)..(1142)
atcctactcgaggcctttggagaatgtaatatcccttgatcaccccagactcacatccagattctgactgacctcgtgcatagccatatttaacttggtccctgatgatcaaaacagtacatggaactggggccgaacatttccctagccaaaggagcatcagggattcaacgtgatcagtacagccttgtagctcgctgaaactgccctggagccaagccaaagcctagacagcaaggtgcatgggcgagctttgcgaggatttagttgttatttagtatgccaagattaagccacgcagaatggcttatggccatagtagcaacaacggccacggaacgttcttccatgtggtatggggaaagccgccggatatttggggagggcgtgattgttccggtggctatctttccggtggagataacggcggtggaccatcaaaactcctcagcccggattgctcggactccgcatcgtcaagtatcggtttcatgacagccattcgactacttagaagacacaaatagctatccaacttacccggacctttgataataattttcagcagatctgtcgatctcagagacaataatgccaccgttctgtgtggaaggcactgcgccgtggtttaacaataataaaactccaaattgcccaaaagctagagcgaaggaaagtcgagaagcccagtctagactggcgggctgatcgtccggttcacaacctgattgacaaatcatctgtgaccctcgatgttccgcggcgttgagcgggtgagaatgtggatcctagtggggaagacccaattgccgccggaaaaggtgagcttgtgctgcacatgtccggtggctcaattcagggccatgccaggacattgctcttcaaagggatgcgattgctattccctatttacttctttaacccgctgacgggcctgtctggaccgagaattcttggtcaagtttcagtggagcgatccaggaaagaacatccgatatggacagttgccctcgatctcaggtctgtagtcgtgtgacacagaggcctctactcgcttgccttgcatgtcggagctcaaaggtgcgatgtgaattaacacgaggagcgactgtctgtcgcagatgcaacttaattaataggat

SEQ ID NO: 40 - DNA, 39 bp, Artificial Sequence
  primer
acttcattaattaagttgattgaggtagagatgagtttg

SEQ ID NO: 41 - DNA, 35 bp, Artificial Sequence
  primer
acttcactcgagataatgtaggcgacaaagtagcc

SEQ ID NO: 42 - DNA, 36 bp, Artificial Sequence
  primer
acttcattaattaatttgacacttaacgtggtaagg

SEQ ID NO: 43 - DNA, 33 bp, Artificial Sequence
  primer
acttcactcgagttctcacaagcaaatccgaga

SEQ ID NO: 44 - DNA, 33 bp, Artificial Sequence
  primer
atcctactcgagtggtgcctgtgaacgaggtca

SEQ ID NO: 45 - DNA, 39 bp, Artificial Sequence
  primer
acttcattaattaatgtggattttggatagtaattaaag

SEQ ID NO: 46 - DNA, 38 bp, Artificial Sequence
  primer
atcctattaattaagatggtttggtgtataacagaaca

SEQ ID NO: 47 - DNA, 34 bp, Artificial Sequence
  primer
acttcactcgaggtccacactctaattgggatga

SEQ ID NO: 48 - DNA, 35 bp, Artificial Sequence
  primer
atcctattaattaagttgcatctgcgacagacagt

SEQ ID NO: 49 - DNA, 35 bp, Artificial Sequence
  primer
acttcactcgaggcctttggagaatgtaatatccc

SEQ ID NO: 50 - DNA, 20 bp, Artificial Sequence
  primer
caacacctacggcgtcaagg

SEQ ID NO: 51 - DNA, 20 bp, Artificial Sequence
  primer
taacccggggagatgctgtt

SEQ ID NO: 52 - DNA, 20 bp, Artificial Sequence
  primer
cgatccggaaattcctcctg

SEQ ID NO: 53 - DNA, 19 bp, Artificial Sequence
  primer
gccgccgactcagcagtat

SEQ ID NO: 54 - DNA, 20 bp, Artificial Sequence
  primer
gtgcatgaacaacacggaga

SEQ ID NO: 55 - DNA, 20 bp, Artificial Sequence
  primer
actcagttgtgcgcctctcg

SEQ ID NO: 56 - DNA, 20 bp, Artificial Sequence
  primer
gtcgaactcggcgtttctcc

SEQ ID NO: 57 - DNA, 20 bp, Artificial Sequence
  primer
ccggtgaggaggtgcttttc

SEQ ID NO: 58 - DNA, 20 bp, Artificial Sequence
  primer
agcatctcgccatccatcag

SEQ ID NO: 59 - DNA, 20 bp, Artificial Sequence
  primer
gccttcggaccatggtatcc

SEQ ID NO: 60 - skipped

SEQ ID NO: 61 - DNA, 1126 bp, Artificial Sequence
  fusion gene
  PRIMER_BIND (1)..(22)
  gene (23)..(739): GFP
  gene (740)..(1212): BLE
gtcctgttaattaaccttcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaaggcgcgcctgaagt

SEQ ID NO: 62 - DNA, 37 bp, Artificial Sequence
  primer
gtcctgttaattaaccttcaccatggtgagcaagggc

SEQ ID NO: 63 - DNA, 36 bp, Artificial Sequence
  primer
gcactggtcaacttggccttgtacagctcgtccatg

SEQ ID NO: 64 - DNA, 36 bp, Artificial Sequence
  primer
catggacgagctgtacaaggccaagttgaccagtgc

SEQ ID NO: 65 - DNA, 35 bp, Artificial Sequence
  primer
acttcaggcgcgcctttagtcctgctcctcggcca